(19) United States
(12) Patent Application Publication
(10) Pub. No.: US 2025/0275852 A1
(43) Pub. Date: Sep. 4, 2025

(54) Transcatheter Prosthetic Venous Valve Assemblies and Associated Catheter Delivery Systems and Methods

(71) Applicant: Inari Medical, Inc., Irvine, CA (US)

(72) Inventors: Philippe Marchand, Pointe-Claire (CA); John Coleman Thress, Oceanside, CA (US); Brian E. Merritt, San Clemente, CA (US); Marek Lhotak, Torrance, CA (US); Payam Saffari, Aliso Viejo, CA (US); Benjamin James Smith, Costa Mesa, CA (US); Michael J. Girard, Lino Lakes, MN (US)

(21) Appl. No.: 19/067,875

(22) Filed: Mar. 1, 2025

Related U.S. Application Data
(60) Provisional application No. 63/560,724, filed on Mar. 3, 2024.

(51) Int. Cl. A61F 2/24; A61L 27/16; A61L 27/18

(52) U.S. Cl. CPC A61F 2/2418; A61L 27/165; A61L 27/18; A61F 2250/0036

(57) ABSTRACT

Single and dual frame prosthetic venous valve assemblies, and catheter delivery systems and methods, are disclosed. A representative dual frame venous valve assembly includes an outer valve frame having a lumen and a frame and venous valve assembly arranged within the lumen. The frame and venous valve assembly, which also may be a stand-alone prosthetic venous valve assembly, includes an inner valve frame having a frame matrix forming lateral valve supports and valve commissure supports; a polymeric frame coating encapsulating the frame matrix; and a one or more polymeric leaflets integrally formed with or coupled to the polymeric frame coating, with each leaflet moveable between an open or partially open state and a closed or partially closed state. A delivery catheter includes keys or slots for insertion of locking tabs of the outer valve frame, and a deployment control handle for controlling deployment of the venous valve assembly.

### CROSS-REFERENCE TO A RELATED APPLICATION

This application is a nonprovisional of and claims priority to and the benefit of U.S. Provisional Patent Application No. 63/560,724, filed Mar. 3, 2024, inventors Marek Lhotak et al., titled “Transcatheter Prosthetic Venous Valve Assemblies and Associated Catheter Delivery Systems and Methods”, which is commonly assigned herewith, incorporated herein by reference with the same full force and effect as if set forth in its entirety herein, and with priority claimed for all commonly disclosed subject matter.

### FIELD OF THE INVENTION

The present invention, in general, relates to prosthetic valves, and more particularly, relates to a prosthetic venous valve assembly and associated catheter delivery systems and methods for the interventional treatment of chronic venous insufficiency.

### BACKGROUND OF THE INVENTION

Chronic venous insufficiency (“CVI”) (also referred to as chronic deep venous insufficiency (“CDVI”)) is a venous disorder, which may affect lower extremities in humans for example, in which one or more veins becomes distended and/or one or more venous valves becomes damaged. This distention of a vein may cause a lack of venous valve leaflet coaptation, such that the leaflets of the venous valve do not close sufficiently to prevent reflux of blood and keep blood flowing in the correct direction back to the heart. In other cases, such chronic venous insufficiency may be caused by a deep vein thrombosis (or phlebitis), in which a blood clot and/or associated post-thrombosis scar tissue has formed and damaged the vein and/or associated venous valve.

Overall, chronic venous insufficiency affects about one in twenty adults. Chronic venous insufficiency slows down flow and makes it harder for blood to flow from a subject's lower extremity back up to the heart. CVI may have a cascading effect, causing blood to pool in leg veins, leading to high pressure and enlargement in those veins, venous valve insufficiency, thrombosis, and edema. Without treatment, CVI may also raise the pressure in the leg veins sufficiently high to cause blood vessels, such as capillaries, to burst. In turn, such burst capillaries may create additional problems, including tissue inflammation, tissue damage, tissue discoloration, venous stasis ulcers, infection, cellulitis, and other symptoms.

Treatment for CVI may vary with the severity of the venous insufficiency, and nonsurgical treatments may include lifestyle changes such as exercise and leg elevation, compression therapy, medications such as anticoagulants and antibiotics, sclerotherapy, and endovenous thermal ablation. Various surgical treatments have also been utilized, including ligation, venous stripping, vein bypass, subfascial endoscopic perforator surgery, and phlebectomy, for example and without limitation.

While these various medical and surgical treatments may provide some symptom relief, these various medical and surgical treatments, however, do not cure the venous insufficiency, do not actually restore the functionality of a venous valve and, in many instances, do not actually improve the return circulation of blood from a lower extremity to the heart.

Accordingly, a need remains for prosthetic venous valve assembly and a catheter delivery system and method which can provide the functionality of a venous valve and improve the return circulation of blood from a lower extremity to the heart, while minimizing or eliminating potential thrombus formation. Such a prosthetic venous valve assembly should be capable of being inserted into a subject using a minimally invasive procedure, such as a percutaneous transcatheter procedure. In addition, such a prosthetic venous valve assembly should be comprised of biocompatible materials which are benign and anti-thrombotic, and have sufficient longevity over its expected duty cycle to continue to perform sufficiently without requiring repeated replacement.

### SUMMARY OF THE INVENTION

The exemplary or representative embodiments of the present invention provide numerous advantages. Various representative embodiments provide a fully synthetic polymer transcatheter venous valve, referred to equivalently as a prosthetic venous valve assembly or a transcatheter, prosthetic venous valve assembly. Various representative embodiments provide a prosthetic venous valve assembly and a catheter delivery system and method which can provide the functionality of a venous valve and improve the return circulation of blood from a lower extremity to the heart, while minimizing or eliminating potential thrombus formation. The various representative embodiments of the prosthetic venous valve assembly are capable of being inserted into a subject vein using a minimally invasive procedure, such as a percutaneous transcatheter procedure. In addition, the various representative embodiments of the prosthetic venous valve assembly are comprised of biocompatible materials which are benign and anti-thrombotic, and have sufficient longevity over the expected duty cycle of the valve to continue to perform sufficiently without requiring repeated replacement.

Two main types of representative embodiments of prosthetic venous valve assemblies are illustrated, namely: (1) prosthetic venous valve assemblies having dual valve frames (an outer valve frame and an inner valve frame); and (2) prosthetic venous valve assemblies having a single valve frame. Stated another way, the representative embodiments of the frame and venous valve assemblies described below also may be utilized directly as prosthetic venous valve assemblies, without an outer frame, in addition to the other single frame prosthetic venous valve assemblies illustrated and described below.

Various representative embodiments of a delivery catheter are also disclosed which enable minimally invasive deployment of representative embodiments of one or more prosthetic venous valve assemblies during a single procedure. Advantageously, the representative delivery catheter embodiments provide significant user control over the positioning and deployment of the one or more prosthetic venous valve assemblies in one or more subject veins in the same procedure, without requiring multiple procedures, such as multiple insertions of multiple catheters to deploy multiple valves. The representative embodiments of a delivery catheter are also suitable for concurrent intravenous ultrasound imaging.

It should be noted that in addition to the enumerated claims herein, any and all of the various representative embodiments, as described herein, including the various representative embodiments for the prosthetic venous valve assemblies, the various representative embodiments for the catheters, the various representative embodiments for the methods of deployment, and any and all of their various features and elements described herein, are all within the scope of this disclosure and may be included as claims or within claims, including, for example and without limitation, in the present application and also in continuing and divisional applications. No embodiments, elements, or features are disclaimed in any way or form.

A representative embodiment of a prosthetic venous valve assembly, such as a transcatheter, prosthetic venous valve assembly, is disclosed and comprises: a valve frame comprising a plurality of struts forming a frame matrix having a plurality of closed cells and a valve frame lumen, the plurality of struts further forming a plurality of lateral valve supports and a plurality of valve commissure supports; a polymeric frame coating encapsulating the frame matrix; and a polymeric venous valve having an open or partially open state and a closed or partially closed state, the polymeric venous valve arranged in the lumen, the polymeric venous valve comprising: one or more polymeric leaflets integrally formed with or coupled to the polymeric frame coating, each leaflet of the one or more polymeric leaflets moveable between the open or partially open state and the closed or partially closed state.

Equivalently, the plurality of lateral valve supports and the plurality of valve commissure supports may also be considered to be part of the frame matrix. A representative embodiment of a prosthetic venous valve assembly, such as a transcatheter, prosthetic venous valve assembly, may comprise: a valve frame comprising a plurality of struts forming a frame matrix having a plurality of closed cells, a valve frame lumen, a plurality of lateral valve supports, and a plurality of valve commissure supports; a polymeric frame coating encapsulating the frame matrix; and a polymeric venous valve having an open or partially open state and a closed or partially closed state, the polymeric venous valve arranged in the lumen, the polymeric venous valve comprising: one or more polymeric leaflets integrally formed with or coupled to the polymeric frame coating, each leaflet of the one or more polymeric leaflets moveable between the open or partially open state and the closed or partially closed state.

In any of the various representative embodiments, the polymeric frame coating may have a first thickness encapsulating an outer portion of each valve commissure support of the plurality of valve commissure supports, the first thickness of the polymeric frame coating tapering to a second thickness spaced apart from each valve commissure support of the plurality of valve commissure supports, the second thickness less than the first thickness. In any of the various representative embodiments, the one or more polymeric leaflets each may have at least one third thickness tapering from the second thickness, the at least one third thickness less than the second thickness. In any of the various representative embodiments, the first thickness may be between forty (40) to two hundred fifty (250) microns and the third thickness may be between twenty (20) to eighty (80) microns. Also any of the various representative embodiments, a lateral or side-to-side variation of the first thickness and the third thickness may be between zero (0) to twenty (20) microns. In a representative embodiment, a ratio of the first thickness to the third thickness may be between 5:1 and 1.5:1, or between 4:1 and 2:1. In any of the various representative embodiments, the polymeric frame coating within each closed cell of the plurality of closed cells may have at least one third thickness, the at least one third thickness less than the second thickness

Also any of the various representative embodiments, each strut forming the plurality of valve commissure supports may have a chamfered or beveled edge. In a representative embodiment, each strut forming the plurality of lateral valve supports may have a chamfered or beveled edge. In any of the various representative embodiments, each valve commissure support of the plurality of valve commissure supports may extend proximally from a corresponding apex formed by the plurality of lateral valve supports.

In any of the various representative embodiments, the valve frame may further comprise: a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports. In another representative embodiment, the valve frame may further comprise: a plurality of commissure support strut halos, wherein for each leaflet of the one or more polymeric leaflets, a corresponding commissure support strut halo of the plurality of commissure support strut halos is coupled between and extending proximally from the corresponding valve commissure supports of the plurality of valve commissure supports.

In any of the various representative embodiments, the valve frame may further comprise: at least one locking tab coupled to or integrally formed with and extending distally from the frame matrix. In a representative embodiment, the at least one locking tab is removably insertable into a corresponding key or slot of a delivery catheter.

A representative embodiment of a delivery catheter for deployment of the prosthetic venous valve assembly in a subject vein is also disclosed, with the prosthetic venous valve assembly having a compressed state, with the catheter having a longitudinal dimension and a transverse or radial dimension, and with the catheter having a distal end and a proximal end, and the delivery catheter comprising: a first shaft extending in the longitudinal dimension, the first shaft having at least one corresponding key or slot, the at least one corresponding key or slot comprising a recess configured to receive the at least one locking tab; a second shaft having a second shaft lumen, the first shaft arranged coaxially within the second shaft lumen, the second shaft longitudinally moveable with respect to the first shaft; a capsule coupled to the second shaft, the capsule arranged coaxially with the first shaft, the capsule configured to cover the prosthetic venous valve assembly in the compressed state, and the capsule longitudinally moveable with the second shaft to uncover the prosthetic venous valve assembly for deployment in the subject vein; and a deployment control handle coupled proximally to the second shaft, the deployment control handle configured to advance and to retract the second shaft longitudinally with respect to the first shaft.

A representative embodiment of a method for deployment of the prosthetic venous valve assembly using the catheter is also disclosed, with the prosthetic venous valve assembly being configured to self-expand and release from the first shaft, the method comprising: inserting and positioning the catheter at a first selected or desired vasculature location in the subject vein; using the deployment control handle, retracting the second shaft longitudinally with respect to the first shaft to uncover and deploy the prosthetic venous valve assembly; and following deployment of the prosthetic venous valve assembly, removing the catheter from the subject vein.

Another representative embodiment of a method for deployment of the prosthetic venous valve assembly using the catheter is also disclosed, with the prosthetic venous valve assembly being configured to self-expand and release from the first shaft, the method comprising: inserting and positioning the catheter at a first selected or desired vasculature location in a first subject vein; using the deployment control handle, retracting the second shaft longitudinally with respect to the first shaft to uncover and deploy a first prosthetic venous valve assembly of the plurality of prosthetic venous valve assemblies; positioning the catheter at a second selected or desired vasculature location in the first subject vein or in a second subject vein; using the deployment control handle, retracting the second shaft longitudinally with respect to the first shaft to uncover and deploy a second prosthetic venous valve assembly of the plurality of prosthetic venous valve assemblies; and following deployment of the second prosthetic venous valve assembly, removing the catheter from the first or second subject vein.

In any of the various representative embodiments, each leaflet of the one or more polymeric leaflets is coupled between corresponding valve commissure supports of the plurality of valve commissure supports.

In any of the various representative embodiments, the plurality of struts may further form one or more sinuses. In a representative embodiment, each leaflet of the one or more polymeric leaflets is arranged and moveable within a corresponding sinus of the one or more sinuses. In a representative embodiment, at least one sinus of the one or more sinuses has a spherical shape or a bulbous shape.

In any of the various representative embodiments, the valve frame may further comprise a plurality of integrally formed adaptive, closed cell strut extension members. In any of the various representative embodiments, the valve frame and the polymeric venous valve are radially collapsible and radially expandable.

In any of the various representative embodiments, each leaflet of the one or more polymeric leaflets may have a sinusoidal or scalloped edge for forming a commissure with an adjacent leaflet of the plurality of polymeric leaflets in the closed or partially closed state.

In any of the various representative embodiments, the polymeric frame coating of the polymeric venous valve comprises one or more polymeric layers or sheets adhered to or encapsulating the valve frame. In a representative embodiment, the one or more polymeric leaflets have a bias, the bias selected from the group consisting of: an open bias, a neutral bias, and a closed bias, and combinations thereof. In a representative embodiment, the plurality of valve commissure supports may be triangular-shaped and define a corresponding plurality of openings or gaps.

In any of the various representative embodiments, each leaflet of the one or more polymeric leaflets comprises a flexible polymeric sheet or layer moveable between the open or partially open state and the closed or partially closed state within a corresponding opening or gap of the plurality of openings or gaps.

In any of the various representative embodiments, at least two valve commissure supports of the plurality of valve commissure supports are coupled to form a triangular apex, the triangular apex having an acute angle.

In any of the various representative embodiments, the polymeric valve has a diameter between ten (10) to fourteen (14) mm and wherein a thickness of each leaflet of the one or more polymeric leaflets is between thirty (30) to forty (40) microns. In another representative embodiment, the polymeric valve has a diameter between five (5) to fourteen (14) mm and wherein a thickness of each leaflet of the one or more polymeric leaflets is between twenty (20) to eighty (80) microns.

In any of the various representative embodiments, the one or more polymeric leaflets comprise at least one polymer selected from the group consisting of: a siloxane poly(urethane-urea) elastomer, a silicone-polyurethane copolymer; a polydimethylsiloxane polyurethane (PDMS-PU), a thermoplastic silicone polyether polyurethane (TSPU), a polyhexamethyleneoxide (PHMO), a poly(dimethylsiloxane) (PDMS), a thermoplastic silicone polycarbonate polyurethane (TSPCU), a segmented polyether polyurethane (SPU), a polyhedral oligomeric silsesquioxane poly(carbonate-urea) urethane (POSS-PCU), a nanocomposite graphene-PCU (FGO-PCU, a poly(styrene-b-isobutylene-styrene) (SIBS), a poly(styrene-b-4-vinylbenzocyclobutylene-b-isobutylene-b-styrene-b-4-vinylbencocylcobutene) (xSIBS), a thermoplastic silicone polycarbonate polyurethane (TSPCU), an aliphatic polycarbonate urethane (PCU), a siloxane poly(urethane-urea) (SiPUU), a carbonate polyurethane, a polyether, a polyether polyol, a polyether polyurethane, a polyurethane, a polyurethane urea, a siloxane, a polysiloxanes, silicone, silicone rubber, polytetrafluoroethylene (PTFE), expanded polytetrafluoroethylene (ePTFE), polyvinyl alchohol (PVA), a siloxane-based polyurethane, a silicone polyurethane copolymer, and combinations thereof.

In any of the various representative embodiments, the polymeric valve may have a single leaflet configuration, or a bi-leaflet configuration, or a tri-leaflet configuration. In a representative embodiment, the valve frame may be cylindrically-shaped to form a conduit.

In any of the various representative embodiments, a selected valve commissure support of the plurality of valve commissure supports divides the polymeric frame coating into a first side polymeric frame coating and a second side polymeric frame coating, wherein a variation of thickness between corresponding portions of the first side polymeric frame coating and the second side polymer frame coating is less than or equal to 20 microns.

Also any of the various representative embodiments, the polymeric frame coating conformally encapsulates or coats both an inner portion and an outer portion of each of the frame matrix, the plurality of lateral valve supports, and the plurality of valve commissure supports.

Also any of the various representative embodiments, each valve commissure support of the plurality of valve commissure supports comprises at least one double beveled edge. In a representative embodiment, a thickness of the polymeric frame coating on an outer portion of each valve commissure support of the plurality of valve commissure supports is between 25 microns and 150 microns. In any of the various representative embodiments, one or more struts of the plurality of struts comprise at least one single beveled edge.

Another representative embodiment of a prosthetic venous valve assembly, such as a transcatheter, prosthetic venous valve assembly, is disclosed and comprises: a valve frame comprising a plurality of struts forming a frame matrix having a plurality of closed cells and a valve frame lumen, the plurality of struts further forming a plurality of lateral valve supports and a plurality of valve commissure supports, each valve commissure support of the plurality of valve commissure supports comprising at least one double beveled edge; a polymeric frame coating encapsulating both an inner portion and an outer portion of the frame matrix, the polymeric frame coating having a first thickness encapsulating an outer portion of each valve commissure support of the plurality of valve commissure supports, the first thickness of the polymeric frame coating tapering to a second thickness spaced apart from each valve commissure support of the plurality of valve commissure supports, the second thickness less than the first thickness; and a polymeric venous valve having an open or partially open state and a closed or partially closed state, the polymeric venous valve arranged in the lumen, the polymeric venous valve comprising: one or more polymeric leaflets integrally formed with or coupled to the polymeric frame coating, each leaflet of the one or more polymeric leaflets moveable between the open or partially open state and the closed or partially closed state, the one or more polymeric leaflets each having at least one third thickness tapering from the second thickness, the at least one third thickness less than the second thickness.

In such a representative embodiment, the first thickness is between 25 microns and 150 microns. Also in such a representative embodiment, at least one valve commissure support of the plurality of valve commissure supports divides the polymeric frame coating into a first side polymeric frame coating and a second side polymeric frame coating, wherein a variation of thickness between corresponding portions of the first side polymeric frame coating and the second side polymeric frame coating is greater than or equal to zero (0) microns and less than or equal to twenty (20) microns.

Another representative embodiment of a prosthetic venous valve assembly, such as a transcatheter, prosthetic venous valve assembly, is disclosed and comprises: a valve frame comprising a plurality of struts forming a frame matrix having a plurality of closed cells and a valve frame lumen, the plurality of struts further forming a plurality of lateral valve supports and a plurality of valve commissure supports, each valve commissure support of the plurality of valve commissure supports comprising at least one double-beveled edge, and one or more struts of the plurality of struts having a single-beveled edge, the valve frame further comprising a plurality of commissure support strut halos coupled between corresponding valve commissure supports of the plurality of valve commissure supports, each end of each commissure support strut halo coupled to and extending proximally from a corresponding valve commissure support of the plurality of valve commissure supports; a polymeric frame coating encapsulating both an inner portion and an outer portion of the frame matrix, the polymeric frame coating having a first thickness encapsulating an outer portion of each valve commissure support of the plurality of valve commissure supports, the first thickness of the polymeric frame coating tapering to a second thickness spaced apart from each valve commissure support of the plurality of valve commissure supports, the second thickness less than the first thickness; and a polymeric venous valve having an open or partially open state and a closed or partially closed state, the polymeric venous valve arranged in the lumen, the polymeric venous valve comprising: one or more polymeric leaflets integrally formed with or coupled to the polymeric frame coating, each leaflet of the one or more polymeric leaflets moveable between the open or partially open state and the closed or partially closed state, the one or more polymeric leaflets each having at least one third thickness tapering from the second thickness, the at least one third thickness less than the second thickness.

Another representative embodiment of a prosthetic venous valve assembly, such as a transcatheter, prosthetic venous valve assembly, is disclosed and comprises: a first, outer valve frame having a lumen, the first, outer valve frame comprising: a central frame matrix having a first plurality of closed cells and forming one or more sinuses, the central frame matrix having a proximal end and a distal end; a first plurality of adaptive, closed cell strut extension members integrally formed with the distal end of the central frame matrix; and a second plurality of adaptive, closed cell strut extension members integrally formed with the proximal end of the central frame matrix; and a frame and venous valve assembly arranged within the lumen of the first, outer valve frame, the frame and valve assembly comprising: a second, inner valve frame comprising a second frame matrix having a second plurality of closed cells, the second, inner valve frame forming a plurality of lateral valve supports and a plurality of valve commissure supports; and a polymeric venous valve having an open or partially open state and a closed or partially closed state, the polymeric valve comprising: an inner frame coating encapsulating or otherwise coupled to the second frame matrix; and a plurality of leaflets integrally formed with the inner frame coating, each leaflet of the plurality of leaflets moveable between the open or partially open state and the closed or partially closed state.

In any of the various representative embodiments, the plurality of leaflets are arranged and moveable within the one or more sinuses of the central frame matrix.

In any of the various representative embodiments, each leaflet of the plurality of leaflets is coupled between corresponding valve commissure supports of the plurality of valve commissure supports. In any of the various representative embodiments, the second, inner valve frame further comprises: a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports. In another representative embodiment, the second, inner valve frame further comprises: a plurality of commissure support strut halos, wherein for each leaflet of the plurality of leaflets, a corresponding commissure support strut halo of the plurality of commissure support strut halos is coupled between and extending proximally from the corresponding valve commissure supports of the plurality of valve commissure supports.

In any of the various representative embodiments, the prosthetic venous valve assembly may further comprise: a polymeric coating encapsulating the central frame matrix.

In any of the various representative embodiments, the first, outer valve frame may further comprise: at least one locking tab coupled to or integrally formed with and extending from at least one adaptive, closed cell strut extension member of the first or second pluralities of adaptive, closed cell strut extension members. In such a representative embodiment, the at least one locking tab may be removably insertable into a corresponding key or slot of a delivery catheter.

In any of the various representative embodiments, the first, outer valve frame and second, inner valve frame may be radially (transversely) collapsible and radially (transversely) expandable.

In various representative embodiments, each leaflet of the plurality of leaflets has a sinusoidal or scalloped edge for forming a commissure with an adjacent leaflet of the plurality of leaflets in the closed or partially closed state. In various representative embodiments, the polymeric valve has a bi-leaflet or a tri-leaflet configuration.

In any of the various representative embodiments, the inner frame coating of the polymeric venous valve comprises a polymeric layer conformally encapsulating, adhered to or surrounding the second, inner valve frame. In a representative embodiment, the plurality of leaflets have a bias, the bias selected from the group consisting of: an open bias, a neutral bias, and a closed bias, and combinations thereof.

In any of the various representative embodiments, the plurality of valve commissure supports are triangular-shaped and define a corresponding plurality of openings or gaps. In a representative embodiment, each leaflet of the plurality of leaflets comprises a flexible polymeric layer or sheet moveable between the open or partially open state and the closed or partially closed state within a corresponding opening or gap of the plurality of openings or gaps.

In any of the various representative embodiments, each valve commissure support of the plurality of valve commissure supports has a triangular apex, the triangular apex having an acute angle. In such a representative embodiment, the second, inner valve frame may further comprise: a plurality of commissure support strut halos, each end of each commissure support strut halo coupled to and extending proximally from a triangular apex of a corresponding valve commissure support of the plurality of valve commissure supports.

In any of the various representative embodiments, at least one sinus of the one or more sinuses has a spherical shape or a bulbous shape.

In any of the various representative embodiments, the polymeric valve has a diameter between 10-14 mm and wherein a thickness of each leaflet of the plurality of leaflets is between 30-40 microns. In another representative embodiment, the polymeric valve has a diameter between 5-14 mm and wherein a thickness of each leaflet of the plurality of leaflets is between 20-70 microns.

Another representative embodiment of a prosthetic venous valve assembly, such as a transcatheter, prosthetic venous valve assembly, is disclosed and comprises: a first, outer valve frame having a lumen, the first, outer valve frame comprising: a central frame matrix having a first plurality of closed cells and forming one or more sinuses, the central frame matrix having a proximal end and a distal end; a first plurality of adaptive, closed cell strut extension members integrally formed with the distal end of the central frame matrix; and a second plurality of adaptive, closed cell strut extension members integrally formed with the proximal end of the central frame matrix; and a frame and venous valve assembly arranged within the lumen of the first, outer valve frame, the frame and valve assembly comprising: a second, inner valve frame comprising: a second frame matrix having a second plurality of closed cells, the second, inner frame forming a plurality of lateral valve supports and a plurality of valve commissure supports; and a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports; and a polymeric venous valve having an open or partially open state and a closed or partially closed state, the polymeric valve comprising: an inner frame coating encapsulating or otherwise coupled to the second frame matrix; and a plurality of leaflets integrally formed with the inner frame coating, the plurality of leaflets arranged and moveable within the one or more sinuses of the central frame matrix, each leaflet of the plurality of leaflets is coupled between corresponding valve commissure supports of the plurality of valve commissure supports, and each leaflet of the plurality of leaflets moveable between the open or partially open state and the closed or partially closed state.

Another representative embodiment of a prosthetic venous valve assembly, such as a transcatheter, prosthetic venous valve assembly, is disclosed and comprises: a first, outer valve frame having a lumen, the first, outer valve frame comprising: a central frame matrix having a first plurality of closed cells and forming one or more sinuses, the central frame matrix having a proximal end and a distal end, at least one sinus of the one or more sinuses has a spherical shape or a bulbous shape; a polymeric coating encapsulating the central frame matrix; a first plurality of adaptive, closed cell strut extension members integrally formed with the distal end of the central frame matrix; and a second plurality of adaptive, closed cell strut extension members integrally formed with the proximal end of the central frame matrix; and a frame and venous valve assembly arranged within the lumen of the first, outer valve frame, the frame and valve assembly comprising: a second, inner valve frame comprising: a second frame matrix having a second plurality of closed cells, the second, inner frame forming a plurality of lateral valve supports and a plurality of valve commissure supports; and a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports; and a polymeric venous valve having an open or partially open state and a closed or partially closed state, the polymeric valve comprising: an inner frame coating encapsulating or otherwise coupled to the second frame matrix; and a plurality of leaflets integrally formed with the inner frame coating, the plurality of leaflets arranged and moveable within the one or more sinuses of the central frame matrix, each leaflet of the plurality of leaflets is coupled between corresponding valve commissure supports of the plurality of valve commissure supports, each leaflet of the plurality of leaflets moveable between the open or partially open state and the closed or partially closed state, and each leaflet of the plurality of leaflets has a sinusoidal or scalloped edge.

Another representative embodiment of a prosthetic venous valve assembly, such as a transcatheter, prosthetic venous valve assembly, is disclosed and comprises: a valve frame comprising: a frame matrix having a plurality of closed cells; a plurality of lateral valve supports; a plurality of valve commissure supports; and a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports; and a polymeric venous valve having an open or partially open state and a closed or partially closed state, the polymeric valve comprising: a polymeric conduit forming an inner frame coating encapsulating or otherwise coupled to the frame matrix; and a plurality of leaflets integrally formed with the polymeric conduit, each leaflet of the plurality of leaflets is coupled between corresponding valve commissure supports of the plurality of valve commissure supports, each leaflet of the plurality of leaflets moveable between the open or partially open state and the closed or partially closed state.

Another representative embodiment of a prosthetic venous valve assembly, such as a transcatheter, prosthetic venous valve assembly, is disclosed and comprises: a first, outer valve frame having a lumen, the first, outer valve frame comprising: a first plurality of struts forming a central frame matrix having a first plurality of closed cells, the central frame matrix having a proximal end and a distal end; the first plurality of struts further arranged to form a plurality of adaptive, closed cell strut extension members integrally formed with the distal or proximal end of the central frame matrix; and a frame and venous valve assembly arranged within the lumen of the first, outer valve frame, the frame and valve assembly comprising: a second, inner valve frame comprising a second plurality of struts forming a second frame matrix having a second plurality of closed cells, the second plurality of struts further forming a plurality of lateral valve supports and a plurality of valve commissure supports; and a polymeric venous valve having an open or partially open state and a closed or partially closed state, the polymeric valve comprising: an inner frame coating encapsulating or otherwise coupled to the second frame matrix; and one or more polymeric leaflets integrally formed with the inner frame coating, each leaflet of the one or more polymeric leaflets moveable between the open or partially open state and the closed or partially closed state. In a representative embodiment, the polymeric valve has a single leaflet configuration, or a bi-leaflet configuration, or a tri-leaflet configuration.

In any of the various representative embodiments, the first plurality of struts are further arranged to form one or more sinuses in the first, outer valve frame. In such a representative embodiment, each leaflet of the one or more polymeric leaflets is arranged and moveable within a corresponding sinus of the one or more sinuses. In such a representative embodiment, at least one sinus of the one or more sinuses has a spherical shape or a bulbous shape.

Another representative embodiment of a prosthetic venous valve assembly, such as a transcatheter, prosthetic venous valve assembly, is disclosed and comprises: a valve frame comprising a plurality of struts forming a frame matrix having a plurality of closed cells and a valve frame lumen, the plurality of struts further forming a plurality of lateral valve supports and a plurality of valve commissure supports; and a polymeric venous valve having an open or partially open state and a closed or partially closed state, the polymeric venous valve arranged in the lumen, comprising: a polymeric frame coating encapsulating or otherwise coupled to the frame matrix; and one or more polymeric leaflets integrally formed with the polymeric inner frame coating, each leaflet of the one or more polymeric leaflets moveable between the open or partially open state and the closed or partially closed state.

In a representative embodiment, the plurality of struts may further form one or more sinuses. In such a representative embodiment, each leaflet of the one or more polymeric leaflets is arranged and moveable within a corresponding sinus of the one or more sinuses. In such a representative embodiment, at least one sinus of the one or more sinuses has a spherical shape or a bulbous shape.

In another representative embodiment, the valve frame further comprises a plurality of integrally formed adaptive, closed cell strut extension members. In such a representative embodiment, the valve frame may further comprise: at least one locking tab coupled to or integrally formed with and extending from at least one adaptive, closed cell strut extension member of the plurality of integrally formed adaptive, closed cell strut extension members. In such a representative embodiment, the at least one locking tab is removably insertable into a corresponding key or slot of a delivery catheter.

In any of the various representative embodiments, each leaflet of the one or more polymeric leaflets is coupled between corresponding valve commissure supports of the plurality of valve commissure supports.

In any of the various representative embodiments, the valve frame may further comprise: a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports. In any of the various representative embodiments, the valve frame further comprises: a plurality of commissure support strut halos, wherein for each leaflet of the one or more polymeric leaflets, a corresponding commissure support strut halo of the plurality of commissure support strut halos is coupled between and extending proximally from the corresponding valve commissure supports of the plurality of valve commissure supports.

In any of the various representative embodiments, the polymeric frame coating of the polymeric venous valve comprises a polymeric layer encapsulating, adhered to or surrounding the valve frame.

In any of the various representative embodiments, the one or more polymeric leaflets have a bias, the bias selected from the group consisting of: an open bias, a neutral bias, and a closed bias, and combinations thereof.

In a representative embodiment, the plurality of valve commissure supports are triangular-shaped and define a corresponding plurality of openings or gaps. In such a representative embodiment, each leaflet of the one or more polymeric leaflets comprises a flexible polymeric layer or sheet moveable between the open or partially open state and the closed or partially closed state within a corresponding opening or gap of the plurality of openings or gaps.

Representative embodiments of a catheter for deployment of a prosthetic venous valve assembly in a subject vein are also disclosed, with the prosthetic venous valve assembly having a compressed state and an expanded state, the prosthetic venous valve assembly having one or more locking tabs, the catheter having a longitudinal dimension and a transverse or radial dimension, and with the catheter having a distal end and a proximal end. A representative embodiment of a catheter may comprise: a first shaft extending in the longitudinal dimension, the first shaft having one or more keys or slots, each key or slot of the one or more keys or slots comprising a recess configured to receive a corresponding locking tab of the one or more locking tabs of the prosthetic venous valve assembly; a second shaft having a second shaft lumen, the first shaft arranged coaxially within the second shaft lumen, the second shaft longitudinally moveable with respect to the first shaft; and a capsule coupled to the second shaft, the capsule arranged coaxially with the first shaft, the capsule configured to cover the prosthetic venous valve assembly in the compressed state, and the capsule longitudinally moveable with the second shaft to uncover the prosthetic venous valve assembly for deployment in the subject vein.

In a representative embodiment, the capsule may comprise: a capsule locking ring coupled to and moveable with the second shaft, the capsule locking ring arranged radially around and spaced apart from the first shaft, the capsule locking ring further moveable to cover the one or more locking tabs when the one or more locking tabs are inserted into the one or more keys or slots; and a capsule cover arranged radially around and spaced apart from the first shaft, the capsule cover coupled to the capsule locking ring, the capsule cover moveable to cover the prosthetic venous valve assembly in the compressed state. In a representative embodiment, the capsule cover comprises a flexible or elastic polymer.

In a representative embodiment, the catheter may further comprise: a deployment control handle coupled proximally to the second shaft, the deployment control handle configured to advance and to retract the second shaft longitudinally with respect to the first shaft.

In a representative embodiment, the deployment control handle may comprise: a housing; a linear, rack gear arranged in the housing, the linear, rack gear coupled to the second shaft; a circular, pinion gear arranged in the housing and engaged with the linear, rack gear, the linear, rack gear longitudinally moveable in response to rotational movement of the circular, pinion gear; and a drive grip partially arranged in the housing, the drive grip comprising a coaxial drive gear arranged in the housing and engaged with the circular, pinion gear, the circular, pinion gear rotationally moveable in response to rotational movement of the drive grip having the drive gear. In a representative embodiment, the drive grip and the housing each further comprise indicia calibrated to indicate a state or degree of deployment of the prosthetic venous valve assembly.

A representative embodiment of a method of using this representative catheter embodiment is also disclosed, wherein the prosthetic venous valve assembly is configured to self-expand and release from the first shaft, with the representative method comprising: inserting and positioning the catheter at a first selected or desired vasculature location in the subject vein; using the drive grip, retracting the second shaft longitudinally with respect to the first shaft to uncover and deploy the prosthetic venous valve assembly; and following deployment of the prosthetic venous valve assembly, removing the catheter from the subject vein.

Another representative embodiment of a method of using this representative catheter embodiment is also disclosed, with the method comprising: inserting and positioning the catheter at a first selected or desired vasculature location in the subject vein; using the drive grip, retracting the second shaft longitudinally with respect to the first shaft to uncover and deploy the first prosthetic venous valve assembly; positioning the catheter at a second selected or desired vasculature location in the subject vein; using the drive grip, retracting the second shaft longitudinally with respect to the first shaft to uncover and deploy the second prosthetic venous valve assembly; and following deployment of the second prosthetic venous valve assembly, removing the catheter from the subject vein.

In another representative embodiment, the deployment control handle comprises: a housing; a lead screw arranged in the housing, the lead screw coupled to the second shaft, the lead screw having a first plurality of screw threads; and a rotating collar partially arranged in the housing and further arranged coaxially with the lead screw, the rotating collar having a mating, second plurality of screw threads engaged with the first plurality of screw threads, the lead screw longitudinally moveable in response to rotational movement of the rotating collar. In a representative embodiment, the rotating collar and the housing each further comprise indicia calibrated to indicate a state or degree of deployment of the prosthetic venous valve assembly.

A representative embodiment of a method of using this representative catheter embodiment is also disclosed, also wherein the prosthetic venous valve assembly is configured to self-expand and release from the first shaft, with the method comprising: inserting and positioning the catheter at a first selected or desired vasculature location in the subject vein; using the rotating collar, retracting the second shaft longitudinally with respect to the first shaft to uncover and deploy the prosthetic venous valve assembly; and following deployment of the prosthetic venous valve assembly, removing the catheter from the subject vein.

Another representative embodiment of a method of using this representative catheter embodiment is also disclosed, with the method comprising: inserting and positioning the catheter at a first selected or desired vasculature location in the subject vein; using the rotating collar, retracting the second shaft longitudinally with respect to the first shaft to uncover and deploy the first prosthetic venous valve assembly; positioning the catheter at a second selected or desired vasculature location in the subject vein; using the rotating collar, retracting the second shaft longitudinally with respect to the first shaft to uncover and deploy the second prosthetic venous valve assembly; and following deployment of the second prosthetic venous valve assembly, removing the catheter from the subject vein.

In a representative embodiment, the catheter may further comprise: a smooth, atraumatic guiding cone coupled distally to the first shaft. In a representative embodiment, the first shaft may further comprise a first shaft lumen, and the catheter may further comprise: an array of a plurality of ultrasound transducers coupled to the smooth, atraumatic guiding cone; and a plurality of electrical wires coupled to the array of the plurality of ultrasound transducers, the plurality of electrical wires arranged in the first shaft lumen, and the plurality of electrical wires comprising one or more signal wires, a power wire, and a ground wire.

In a representative embodiment, the catheter may further comprise: a third shaft having a third shaft lumen, wherein the second shaft together with the first shaft arranged in the second shaft lumen are collectively arranged coaxially within the third shaft lumen, the third shaft longitudinally moveable with respect to the second shaft.

Another representative embodiment of a catheter may comprise: a first shaft extending in the longitudinal dimension, the first shaft having one or more keys or slots, each key or slot of the one or more keys or slots comprising a recess configured to receive a corresponding locking tab of the one or more locking tabs of the prosthetic venous valve assembly; a second shaft having a second shaft lumen, the first shaft arranged coaxially within the second shaft lumen, the second shaft longitudinally moveable with respect to the first shaft; a capsule coupled to the second shaft, the capsule arranged coaxially with the first shaft, the capsule configured to cover the prosthetic venous valve assembly in the compressed state, and the capsule longitudinally moveable with the second shaft to uncover the prosthetic venous valve assembly for deployment in the subject vein; and a deployment control handle coupled proximally to the second shaft, the deployment control handle configured to advance and to retract the second shaft longitudinally with respect to the first shaft.

A representative embodiment of a method of using this representative catheter embodiment is also disclosed, with the method comprising: inserting and positioning the catheter at a first selected or desired vasculature location in the subject vein; using the deployment control handle, retracting the second shaft longitudinally with respect to the first shaft to uncover and deploy the prosthetic venous valve assembly; and following deployment of the prosthetic venous valve assembly, removing the catheter from the subject vein.

Another representative embodiment of a method of using this representative catheter embodiment is also disclosed, with the method comprising: inserting and positioning the catheter at a first selected or desired vasculature location in the subject vein; using the deployment control handle, retracting the second shaft longitudinally with respect to the first shaft to uncover and deploy the first prosthetic venous valve assembly; positioning the catheter at a second selected or desired vasculature location in the subject vein; using the deployment control handle, retracting the second shaft longitudinally with respect to the first shaft to uncover and deploy the second prosthetic venous valve assembly; and following deployment of the second prosthetic venous valve assembly, removing the catheter from the subject vein.

Another representative embodiment of a catheter may comprise: a first shaft extending in the longitudinal dimension, the first shaft having one or more keys or slots, each key or slot of the one or more keys or slots comprising a recess configured to receive a corresponding locking tab of the one or more locking tabs of the prosthetic venous valve assembly; a second shaft having a second shaft lumen, the first shaft arranged coaxially within the second shaft lumen, the second shaft longitudinally moveable with respect to the first shaft; a capsule coupled to the second shaft, the capsule arranged coaxially with the first shaft, the capsule configured to cover the prosthetic venous valve assembly in the compressed state, the capsule longitudinally moveable with the second shaft to uncover the prosthetic venous valve assembly for deployment in the subject vein, the capsule comprising: a capsule locking ring coupled to and moveable with the second shaft, the capsule locking ring arranged radially around and spaced apart from the first shaft, the capsule locking ring further moveable to cover the one or more locking tabs when the one or more locking tabs are inserted into the one or more keys or slots; and a capsule cover arranged radially around and spaced apart from the first shaft, the capsule cover coupled to the capsule locking ring, the capsule cover moveable to cover the prosthetic venous valve assembly in the compressed state; and a deployment control handle coupled proximally to the second shaft, the deployment control handle configured to advance and to retract the second shaft longitudinally with respect to the first shaft, the deployment control handle comprising: a housing; a linear, rack gear arranged in the housing, the linear, rack gear coupled to the second shaft; a circular, pinion gear arranged in the housing and engaged with the linear, rack gear, the linear, rack gear longitudinally moveable in response to rotational movement of the circular, pinion gear; and a drive grip partially arranged in the housing, the drive grip comprising a coaxial drive gear arranged in the housing and engaged with the circular, pinion gear, the circular, pinion gear rotationally moveable in response to rotational movement of the drive grip having the drive gear.

Another representative embodiment of a catheter may comprise: a first shaft extending in the longitudinal dimension, the first shaft having one or more keys or slots, each key or slot of the one or more keys or slots comprising a recess configured to receive a corresponding locking tab of the one or more locking tabs of the prosthetic venous valve assembly; a second shaft having a second shaft lumen, the first shaft arranged coaxially within the second shaft lumen, the second shaft longitudinally moveable with respect to the first shaft; a capsule coupled to the second shaft, the capsule arranged coaxially with the first shaft, the capsule configured to cover the prosthetic venous valve assembly in the compressed state, the capsule longitudinally moveable with the second shaft to uncover the prosthetic venous valve assembly for deployment in the subject vein, the capsule comprising: a capsule locking ring coupled to and moveable with the second shaft, the capsule locking ring arranged radially around and spaced apart from the first shaft, the capsule locking ring further moveable to cover the one or more locking tabs when the one or more locking tabs are inserted into the one or more keys or slots; and a capsule cover arranged radially around and spaced apart from the first shaft, the capsule cover coupled to the capsule locking ring, the capsule cover moveable to cover the prosthetic venous valve assembly in the compressed state; and a deployment control handle coupled proximally to the second shaft, the deployment control handle configured to advance and to retract the second shaft longitudinally with respect to the first shaft, the deployment control handle comprising: a housing; a lead screw arranged in the housing, the lead screw coupled to the second shaft, the lead screw having a first plurality of screw threads; and a rotating collar partially arranged in the housing and further arranged coaxially with the lead screw, the rotating collar having a mating, second plurality of screw threads engaged with the first plurality of screw threads, the lead screw longitudinally moveable in response to rotational movement of the rotating collar.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 is an isometric view illustrating a representative first embodiment of a prosthetic venous valve assembly in accordance with the disclosure herein.

FIGS. 2A, 2B, 2C, and 2D (collectively referred to as FIG. 2) are partial isometric views illustrating in greater detail the representative first embodiment of the prosthetic venous valve assembly in accordance with the disclosure herein: in FIG. 2A, a partial isometric view illustrating a representative first embodiment of an outer valve frame; in FIG. 2B, a partial, enlarged isometric view illustrating the struts forming the outer valve frame; in FIG. 2C, a partial isometric view illustrating a representative first embodiment of a frame and venous valve assembly (and a third embodiment of a prosthetic venous valve assembly); and in FIG. 2D, a partial, enlarged isometric view illustrating the struts forming an inner valve frame.

FIG. 3 is an isometric, cut-away view illustrating the representative first embodiment of a prosthetic venous valve assembly and first embodiment of a frame and venous valve assembly in accordance with the disclosure herein.

FIG. 4 is an isometric view illustrating a representative second embodiment of a prosthetic venous valve assembly in accordance with the disclosure herein.

FIG. 5 is an isometric, cut-away view illustrating the representative second embodiment of the prosthetic venous valve assembly and a second embodiment of a frame and venous valve assembly (and a fourth embodiment of a prosthetic venous valve assembly) in accordance with the disclosure herein.

FIG. 6 is an isometric view illustrating a representative third embodiment of a prosthetic venous valve assembly and also illustrating in greater detail the representative first embodiment of the frame and venous valve assembly having an inner valve frame and a venous valve in accordance with the disclosure herein.

FIG. 7 is a cross-sectional view (through the A-A′ plane of FIG. 6) of the representative first embodiment of the frame and venous valve assembly having the inner valve frame and the venous valve in accordance with the disclosure herein.

FIG. 8 is a cross-sectional view (through the B-B′ plane of FIG. 6) of the representative first embodiment of the frame and venous valve assembly having the inner valve frame and the venous valve in accordance with the disclosure herein.

FIG. 9 is an isometric view illustrating a representative fifth embodiment of a prosthetic venous valve assembly and also illustrating a representative fourth embodiment of a frame and venous valve assembly having an inner valve frame and a venous valve in accordance with the disclosure herein.

FIG. 10 is an isometric view illustrating the representative sixth embodiment of a prosthetic venous valve assembly and also illustrating the representative fourth embodiment of a frame and venous valve assembly having an inner valve frame and a venous valve in accordance with the disclosure herein.

FIG. 11 is an isometric view illustrating a representative sixth embodiment of a prosthetic venous valve assembly and also illustrating the representative fourth embodiment of a frame and venous valve assembly having an inner valve frame and a venous valve in accordance with the disclosure herein.

FIG. 12 is a side or elevational view illustrating a representative second embodiment of an outer valve frame in accordance with the disclosure herein.

FIG. 13 is a partial, side or elevational view illustrating a representative third embodiment of an outer valve frame with a central coating in accordance with the disclosure herein.

FIG. 14 is a side or elevational view illustrating the representative second embodiment of an outer valve frame with a central coating and a plurality of locking tabs in accordance with the disclosure herein.

FIG. 15 is a side or elevational view illustrating a representative fourth embodiment of an outer valve frame with two individual sinuses (one sinus per valve leaflet) with a central coating in accordance with the disclosure herein.

FIG. 16 is a side or elevational view illustrating a representative fifth embodiment of an outer valve frame with three individual sinuses (one sinus per valve leaflet) with a central coating in accordance with the disclosure herein.

FIG. 17 is a cut-away view illustrating the representative second embodiment of a prosthetic venous valve assembly deployed in situ within a representative vein in accordance with the disclosure herein.

FIG. 18 is a side or elevational view illustrating a representative seventh embodiment of a prosthetic venous valve assembly and also illustrating a representative fifth embodiment of a frame and venous valve assembly in accordance with the disclosure herein.

FIG. 19 is a top view illustrating the representative seventh embodiment of a prosthetic venous valve assembly and also illustrating the representative fifth embodiment of a frame and venous valve assembly in accordance with the disclosure herein.

FIG. 20 is a side or elevational view illustrating a representative eighth embodiment of a prosthetic venous valve assembly and also illustrating a representative sixth embodiment of a frame and venous valve assembly in accordance with the disclosure herein.

FIG. 21 is a cut-away view illustrating the representative eighth embodiment of the prosthetic venous valve assembly and also illustrating the representative sixth embodiment of a frame and venous valve assembly in accordance with the disclosure herein.

FIGS. 22A, 22B, 22C, and 22D (collectively referred to as FIG. 22) are top or plan (outflow) views illustrating respectively: in FIG. 22A, a closed bias; in FIG. 22B, a first neutral bias; in FIG. 22C, a second neutral bias; and in FIG. 22D, an open bias; all of the representative first embodiment of the prosthetic venous valve in accordance with the disclosure herein.

FIG. 23 is a top or plan (outflow) view illustrating an open state or configuration of the representative first embodiment of the prosthetic venous valve in accordance with the disclosure herein.

FIG. 24 is a top or plan (outflow) view illustrating a partially closed state or configuration of the representative first embodiment of the prosthetic venous valve in accordance with the disclosure herein.

FIG. 25 is a top or plan (outflow) view illustrating a closed state or configuration of the representative first embodiment of the prosthetic venous valve in accordance with the disclosure herein.

FIGS. 26A, 26B, 26C, 26D, and 26E are cross-sectional schematic illustrations showing blood flow through the opening and closing states of a representative second embodiment of the prosthetic venous valve assembly in accordance with the disclosure herein, with FIG. 26A showing blood flow through a prosthetic venous valve assembly in a first partially open state, FIG. 26B showing blood flow through the prosthetic venous valve assembly in a second partially open state, FIG. 26C showing blood flow through the prosthetic venous valve assembly in a fully open state, FIG. 26D showing blood flow through the prosthetic venous valve assembly in a closing or partially closed state, and FIG. 26E showing no blood flow through the prosthetic venous valve assembly in a fully closed state.

FIG. 27 is an isometric view illustrating a representative first embodiment of a delivery catheter having a prosthetic venous valve assembly in its crimped or compressed state or configuration in accordance with the disclosure herein.

FIG. 28 is an isometric view illustrating a representative second embodiment of a delivery catheter having a prosthetic venous valve assembly in its crimped or compressed state or configuration in accordance with the disclosure herein.

FIG. 29 is a cross-sectional view (through the C-C′ plane of FIG. 27 and the G-G′ plane of FIG. 28) of the representative first and second embodiments of the delivery catheters having the prosthetic venous valve assembly in its crimped or compressed state or configuration in accordance with the disclosure herein.

FIG. 30 is a cross-sectional view (through the D-D′ plane of FIG. 27 and the F-F′ plane of FIG. 28) of the representative first and second embodiments of the delivery catheters having the prosthetic venous valve assembly in its crimped or compressed state or configuration in accordance with the disclosure herein.

FIG. 31 is a cross-sectional view (through the E-E′ plane of FIG. 27 and the H-H′ plane of FIG. 28) of the representative first and second embodiments of the delivery catheters in accordance with the disclosure herein.

FIG. 32 is a partial isometric, cut-away view illustrating a representative key or slot of the delivery catheter for removable insertion of a prosthetic venous valve assembly in accordance with the disclosure herein.

FIG. 33 is a partial isometric, cut-away view illustrating a representative locking tab of a prosthetic venous valve assembly removably inserted into the key or slot of the delivery catheter in accordance with the disclosure herein.

FIG. 34 is a partial isometric view illustrating a prosthetic venous valve assembly partially expanding from a crimped or compressed state as a capsule cover of the delivery catheter is being retracted for removable insertion of a prosthetic venous valve assembly in accordance with the disclosure herein

FIG. 35 is a partial isometric view illustrating a prosthetic venous valve assembly (having locking tabs arranged proximally) partially expanding from a crimped or compressed state as the capsule cover of the delivery catheter is being retracted and the locking tabs are released from keys or slots for removable insertion of a prosthetic venous valve assembly in accordance with the disclosure herein

FIG. 36 is a partial isometric view illustrating a prosthetic venous valve assembly (having locking tabs arranged distally) continuing to expand from a crimped or compressed state as the capsule cover of the delivery catheter is fully retracted and the locking tabs are released from keys or slots for removable insertion of a prosthetic venous valve assembly in accordance with the disclosure herein

FIG. 37 is a cut-away view illustrating a representative first embodiment of a deployment control handle of the first embodiment of the delivery catheter in accordance with the disclosure herein.

FIG. 38 is an isometric view illustrating a representative second embodiment of a deployment control handle of the second embodiment of a delivery catheter in accordance with the disclosure herein.

FIG. 39 is a cut-away view illustrating the representative second embodiment of the deployment control handle of the second embodiment of a delivery catheter in accordance with the disclosure herein.

FIG. 40 is a flow chart illustrating a representative first method embodiment of using a representative delivery catheter for deployment of a prosthetic venous valve assembly in a subject vein in accordance with the disclosure herein.

FIG. 41 is a flow chart illustrating a representative second method embodiment of using a representative delivery catheter for deployment of a plurality of prosthetic venous valve assemblies in one or more subject veins in accordance with the disclosure herein.

FIG. 42 is a first side or elevational view illustrating a representative ninth embodiment of a prosthetic venous valve assembly and also illustrating a representative seventh embodiment of a frame and venous valve assembly in accordance with the disclosure herein.

FIG. 43 is a second, orthogonal side or elevational view illustrating the representative ninth embodiment of a prosthetic venous valve assembly and also illustrating the representative seventh embodiment of a frame and venous valve assembly in accordance with the disclosure herein.

FIG. 44 is partial cross-sectional view (through the I-I′ plane of FIG. 42) illustrating the representative ninth embodiment of the prosthetic venous valve assembly in accordance with the disclosure herein.

FIG. 45 is partial cross-sectional view (through the K-K′ plane of FIG. 42) illustrating the representative ninth embodiment of the prosthetic venous valve assembly in accordance with the disclosure herein.

FIG. 46 is a partial cut-away view (illustrated distal to the J-J′ plane of FIG. 42) providing a sectional view of the valve edges of the valve leaflets and of the frame coating encapsulating the valve commissure supports, and illustrating a variable thickness of the valve leaflets and a variable thickness of the inner frame coating of the representative ninth embodiment of the prosthetic venous valve assembly in accordance with the disclosure herein.

FIG. 47 is an enlarged view of a portion of the partial cut-away view of FIG. 46 illustrating in greater detail the variable thickness of the inner frame coating and the variable thickness of the valve leaflets of the representative ninth embodiment of the prosthetic venous valve assembly in accordance with the disclosure herein.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

Various representative embodiments provide a prosthetic venous valve assembly and a catheter delivery (or, equivalently, deployment) system and method which can provide the functionality of a venous valve and improve the return circulation of blood from a lower extremity to the heart, while minimizing or eliminating potential thrombus formation. The various representative embodiments of the prosthetic venous valve assembly are capable of being inserted into a subject vein using a minimally invasive procedure, such as a percutaneous transcatheter procedure. In addition, the various representative embodiments of the prosthetic venous valve assembly are comprised of biocompatible materials which are benign and anti-thrombotic, and which have sufficient longevity over the expected duty cycle of the valve to continue to perform sufficiently without requiring repeated replacement.

As used in this application and in the claims, the singular forms “a,” “an,” and “the” include the plural forms unless the context clearly dictates otherwise. Additionally, the term “includes” means “comprises.” Further, the terms “coupled” generally means electrically, electromagnetically, and/or physically (e.g., mechanically or chemically) coupled or linked and does not exclude the presence of intermediate elements between the coupled items.

As used herein, the “expanded” or “deployed” state of a prosthetic venous valve assembly 100, 100A, 200-200F or frame 110-110D, 160-160E refers to the state of the prosthetic venous valve assembly 100, 100A, 200-200F or frame 110-110D, 160-160E when radially expanded to its functional size. The “crimped”, “compressed” or “folded” state of a prosthetic venous valve assembly 100, 100A, 200-200F or frame 110-110D, 160-160E refers to the state of the prosthetic venous valve assembly 100, 100A, 200-200F or frame 110-110D, 160-160E when radially compressed or collapsed (e.g., using a crimper or loading kit, not separately illustrated) to a diameter suitable for delivering the valve assembly through a patient's vasculature on a catheter or equivalent mechanism. “Partially crimped” or “partially compressed” or “partially expanded” means that at least a portion of a prosthetic venous valve assembly 100, 100A, 200-200F or frame 110-110D, 160-160E has a diameter that is less than the diameter of the prosthetic venous valve assembly 100, 100A, 200-200F or frame 110-110D, 160-160E in the fully expanded state and greater than the diameter of the prosthetic venous valve assembly 100, 100A, 200-200F or frame 110-110D, 160-160E in the compressed state within or coupled to the delivery system.

The terms “delivery configuration” and “operating configuration” refer to the arrangement of the components of the prosthetic venous valve assembly 100, 100A, 200-200F relative to one another, and each term includes both crimped and non-crimped (e.g., expanded) states. The term “fully assembled” refers to prosthetic venous valve assemblies 100, 100A, 200-200F in which all required components are coupled together, and thus a prosthetic venous valve assembly can be considered fully assembled in both delivery and operating configurations, even when in a crimped position within a delivery catheter.

Terms such as “above,” “upper,” “below,” and “lower” are meant only to show the position of some features relative to others as shown in the drawings, and do not necessarily correlate to actual positions or directions of those features when the prosthetic venous valve assembly 100, 100A, 200-200F is being delivered and/or is in its implanted configuration or position.

Terms such as “distal” and “proximal” are made with reference to the position of a prosthetic venous valve assembly 100, 100A, 200-200F once inserted into a subject's vein, rather than with respect to the orientation of the prosthetic venous valve assembly 100, 100A, 200-200F within a delivery catheter (or delivery catheter assembly) 300, 300A, with the distal end of the prosthetic venous valve assembly 100, 100A, 200-200F oriented distally within the vein and the proximal end of the prosthetic venous valve assembly 100, 100A, 200-200F oriented proximally within the vein, such that blood will be flowing through the prosthetic venous valve assembly 100, 100A, 200-200F from the distal (inflow) end of the prosthetic venous valve assembly 100, 100A, 200-200F to the proximal (outflow) end of the prosthetic venous valve assembly 100, 100A, 200-200F and back toward the subject's heart.

Descriptions and disclosures provided in association with one particular embodiment are not limited to that embodiment, and may be applied to any embodiment disclosed. Moreover, for the sake of simplicity, the figures may not show the various ways (readily discernible, based on this disclosure, by one of ordinary skill in the art) in which the disclosed system, method, and apparatus can be used in combination with other systems, methods, and apparatuses.

Disclosed embodiments of the prosthetic venous valve assembly 100, 100A, 200-200F can be designed for delivery and implantation using minimally invasive techniques. In representative embodiments, the prosthetic venous valve assembly 100, 100A, 200-200F is radially collapsible and expandable, and may be self-expandable or balloon expandable, for example and without limitation. Also for example and without limitation, representative embodiments of the prosthetic venous valve assembly 100, 100A, 200-200F can be collapsed and/or crimped onto or into a delivery catheter 300, 300A, navigated through a patient's venous vasculature, and expanded (and separated from the delivery catheter 300, 300A) before or during implantation in either a native venous valve site or another region of an affected vein. Alternatively, also for example and without limitation, representative embodiments of the prosthetic venous valve assembly 100, 100A, 200-200F may be collapsed and/or crimped onto or into a delivery catheter, navigated through a patient's venous vasculature, and expanded (and separated from the delivery catheter) using a balloon, before or during implantation in either a native venous valve site or another region of an affected vein.

Two main types of representative embodiments of prosthetic venous valve assemblies 100, 100A, 200-200F are illustrated, namely: (1) prosthetic venous valve assemblies 100, 100A having dual valve frames (outer valve frame 110-110D and an (inner) valve frame 160-160E); and (2) prosthetic venous valve assemblies 200-200F having a single valve frame 160-160E. Stated another way, the representative embodiments of the frame and venous valve assemblies 105-105F also may be utilized directly as prosthetic venous valve assemblies 200-200F, without an outer frame 110-110D).

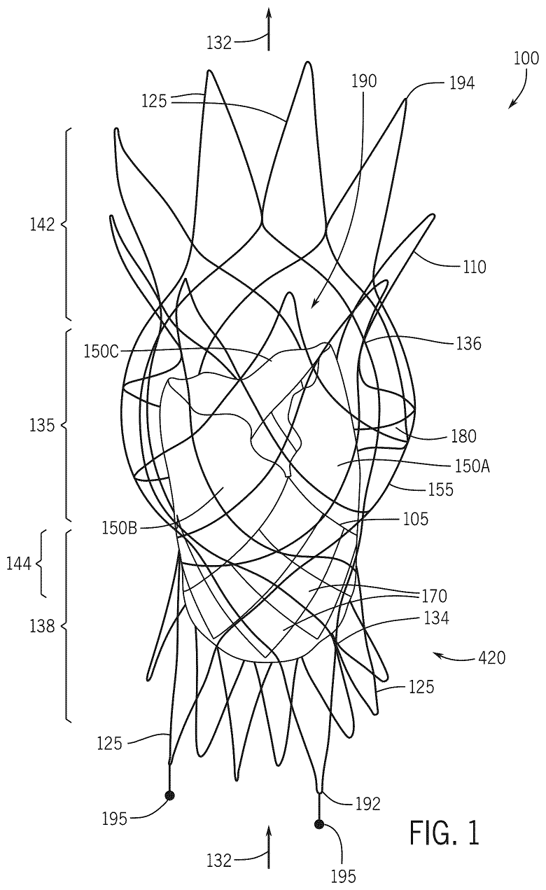

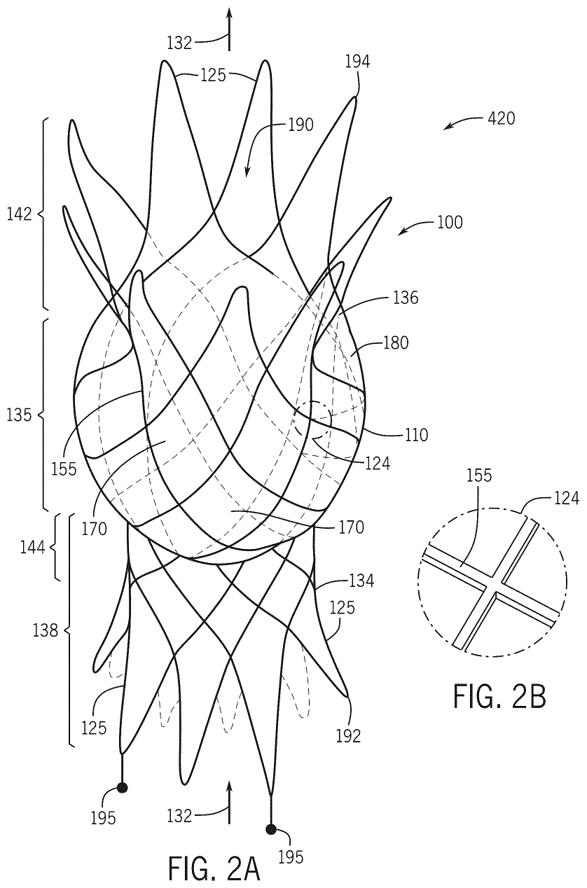

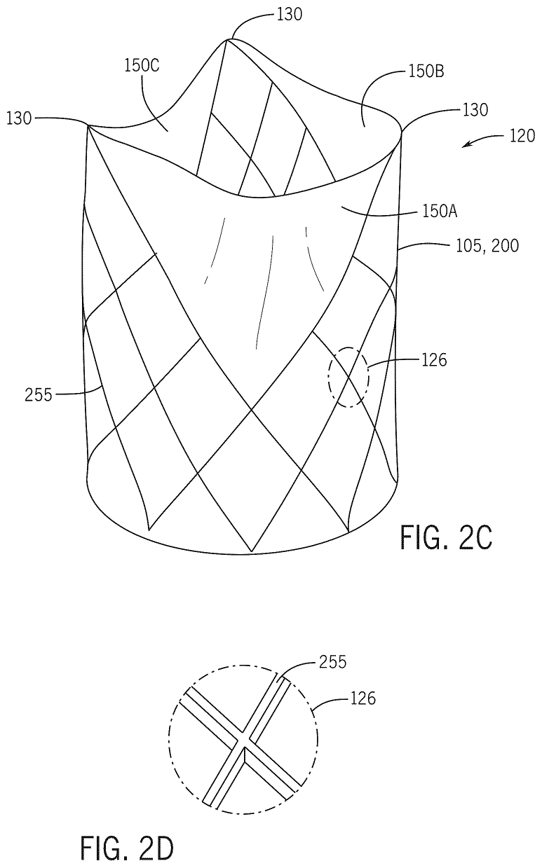

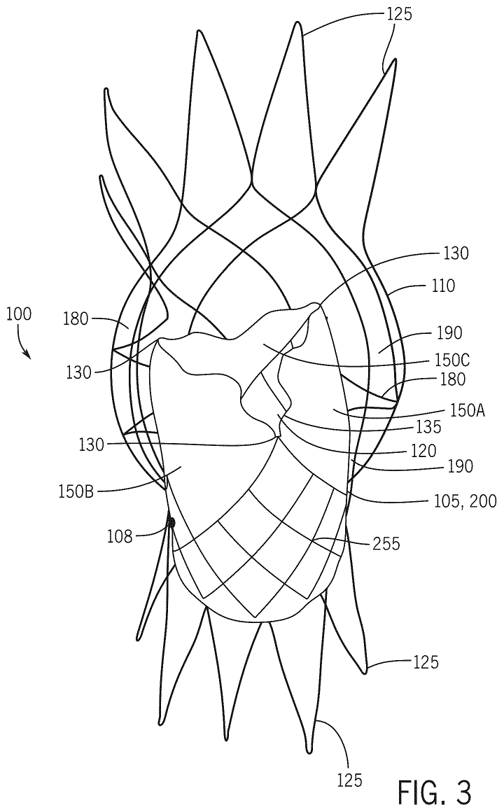

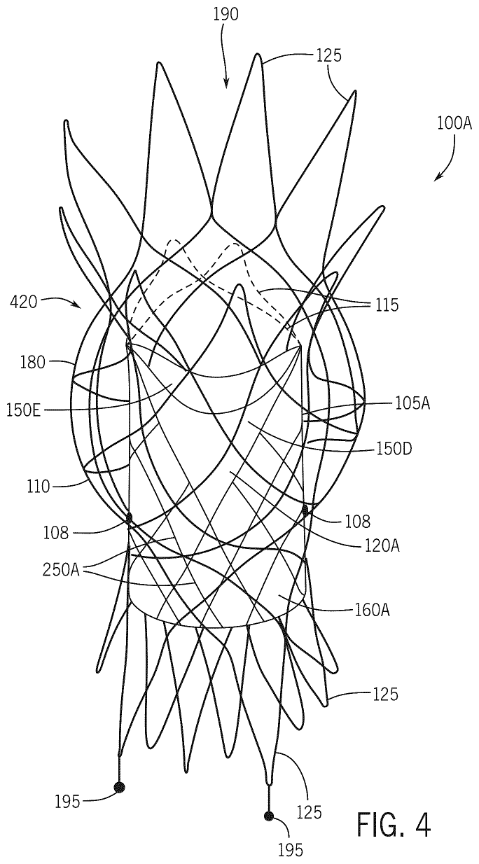

FIG. 1 is an isometric view illustrating a representative first embodiment of a prosthetic venous valve assembly 100 in accordance with the disclosure herein. FIGS. 2A, 2B, 2C, and 2D are partial isometric views illustrating in greater detail a representative first embodiment of an outer valve frame 110 and a representative first embodiment of a frame and venous valve assembly 105 of the representative first embodiment of the prosthetic venous valve assembly 100 in accordance with the disclosure herein, with the dashed lines of FIG. 2A illustrating the rear struts 155 of the outer valve frame 110 to illustrate the frame matrix structure of the outer valve frame 110 with greater clarity. While FIGS. 2A and 2C illustrate the respective struts 155, 255 of the outer valve frame 110 and the frame and venous valve assembly 105, the structures of these respective struts 155, 255 are illustrated in greater detail in FIGS. 2B and 2D, also as described in greater detail below. FIG. 3 is a cut-away view illustrating the representative first embodiment of a prosthetic venous valve assembly 100 and first embodiment of a frame and venous valve assembly 105 in accordance with the disclosure herein. FIG. 4 is an isometric view illustrating a representative second embodiment of a prosthetic venous valve assembly 100A in accordance with the disclosure herein, comprising the outer valve frame 110 with a representative second embodiment of a frame and venous valve assembly 105A. The dashed lines of FIG. 4 illustrate optional commissure support strut (or wire) halos 115 of the frame and venous valve assembly 105A, as described in greater detail below. FIG. 5 is a cut-away view illustrating the representative second embodiment of the prosthetic venous valve assembly 100A in accordance with the disclosure herein. It should be noted, also as described in greater detail below, that the first embodiment of a frame and venous valve assembly 105, when utilized separately from the outer valve frame 110, is also a representative third embodiment of a prosthetic venous valve assembly 200, and the second embodiment of a frame and venous valve assembly 105A, when utilized separately from the outer valve frame 110, is also a representative fourth embodiment of a prosthetic venous valve assembly 200A.

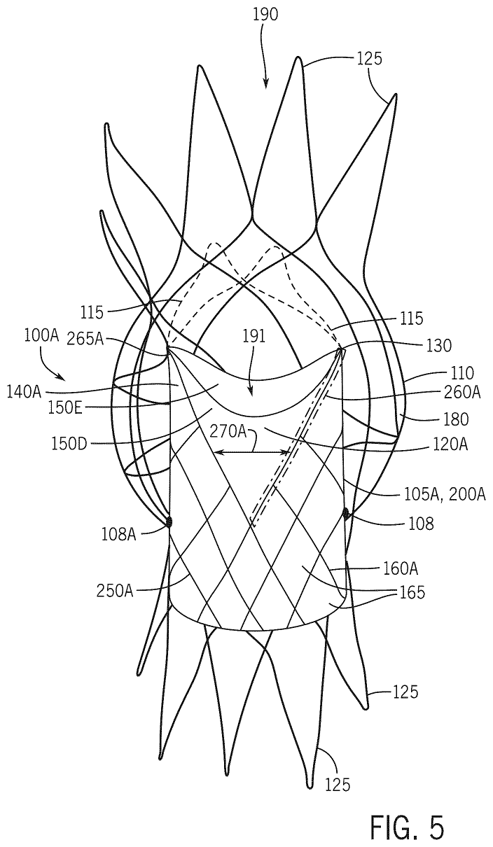

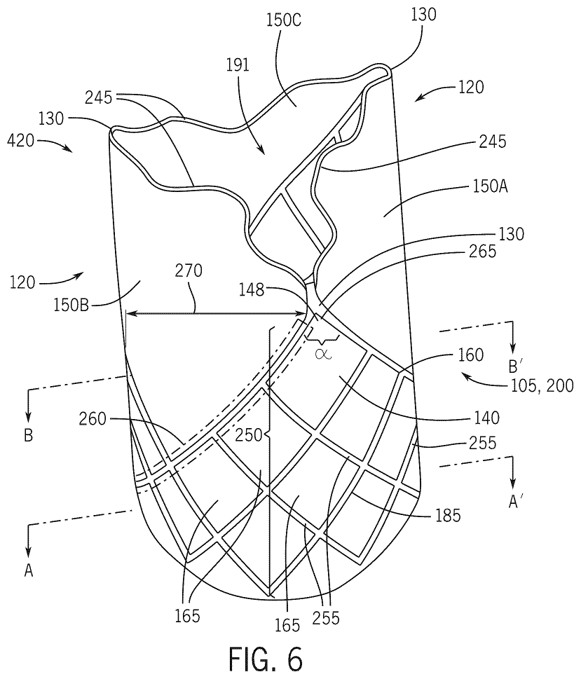

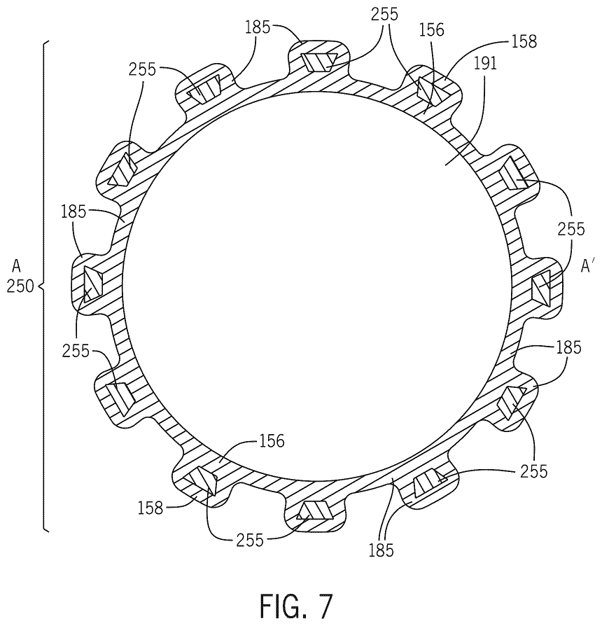

FIG. 6 is an isometric view illustrating a representative third embodiment of a prosthetic venous valve assembly 200 and also illustrating in greater detail the representative first embodiment of a frame and venous valve assembly 105 having a first embodiment of a valve frame 160 (which may also be referred to as an inner valve frame 160) and a first embodiment of a polymeric, prosthetic venous valve 120 (also referred to as a venous valve 120) in accordance with the disclosure herein. FIG. 7 is a cross-sectional view (through the A-A′ plane of FIG. 6) of the representative first embodiment of the frame and venous valve assembly 105 having the (inner) valve frame 160 and the venous valve 120 in accordance with the disclosure herein. FIG. 8 is a cross-sectional view (through the B-B′ plane of FIG. 6) of the representative first embodiment of the frame and venous valve assembly 105 having the (inner) valve frame 160 and the venous valve 120 in accordance with the disclosure herein.

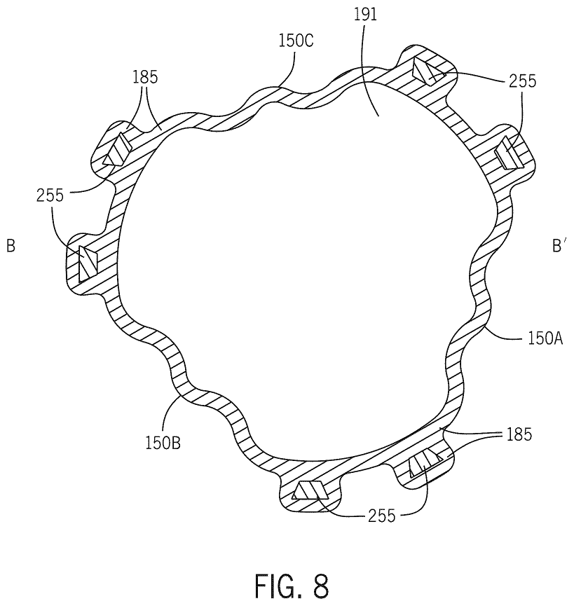

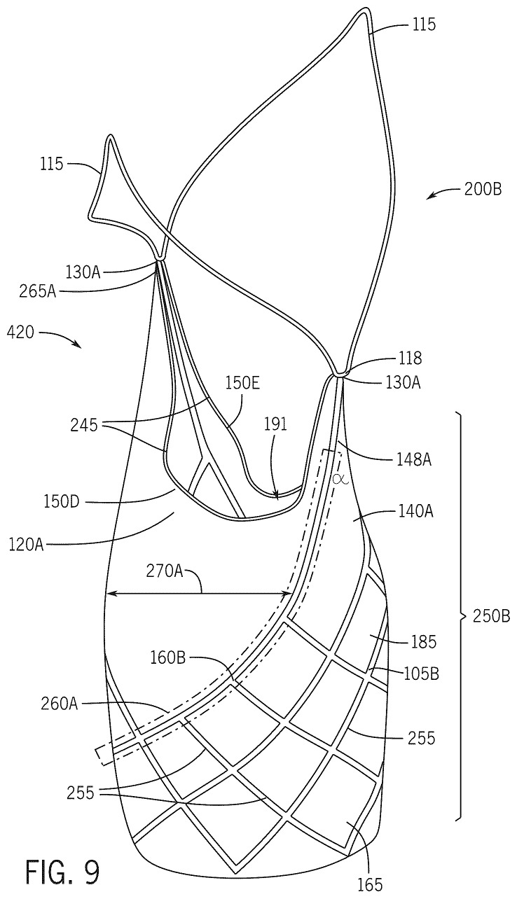

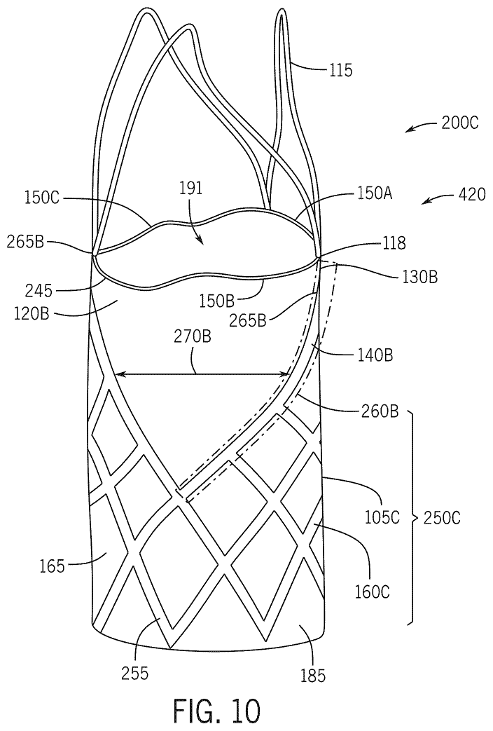

FIG. 9 is an isometric view illustrating a representative fifth embodiment of a prosthetic venous valve assembly 200B and also illustrating a representative third embodiment of a frame and venous valve assembly 105B having a third embodiment of an (inner) valve frame 160B and a second embodiment of a polymeric, prosthetic venous valve 120A (also referred to as a venous valve 120A) in accordance with the disclosure herein. FIG. 10 is an isometric view illustrating a representative sixth embodiment of a prosthetic venous valve assembly 200C and also illustrating a representative fourth embodiment of a frame and venous valve assembly 105C having an (inner) valve frame 160C and a polymeric, prosthetic venous valve 120B in accordance with the disclosure herein. FIG. 11 is an isometric view illustrating the representative sixth embodiment of a prosthetic venous valve assembly 200C and also illustrating the representative fourth embodiment of a frame and venous valve assembly 105C having an (inner) valve frame 160C and a venous valve 120B in accordance with the disclosure herein.

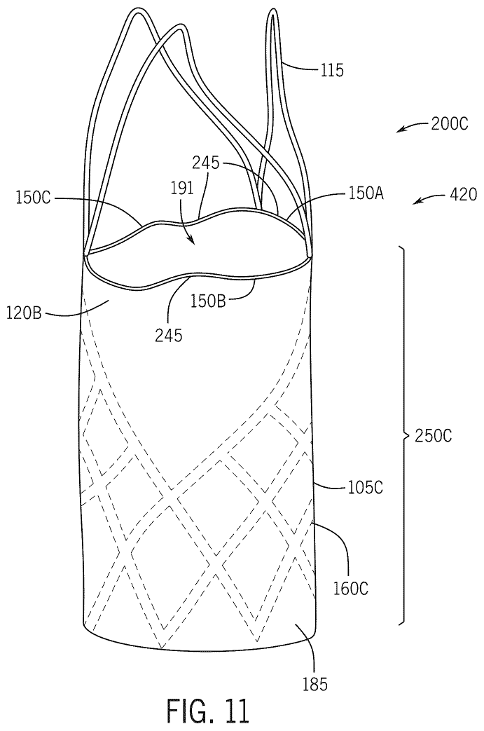

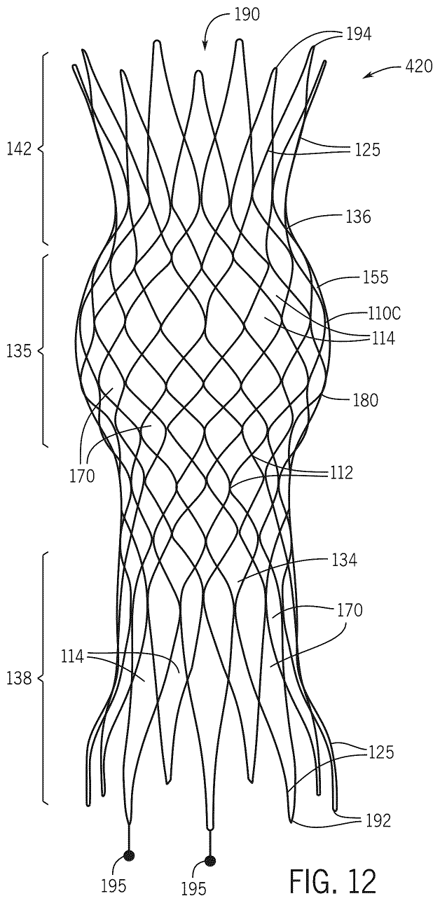

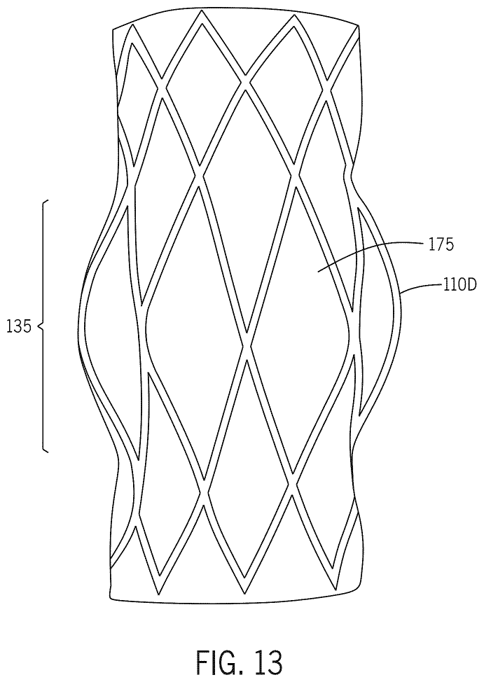

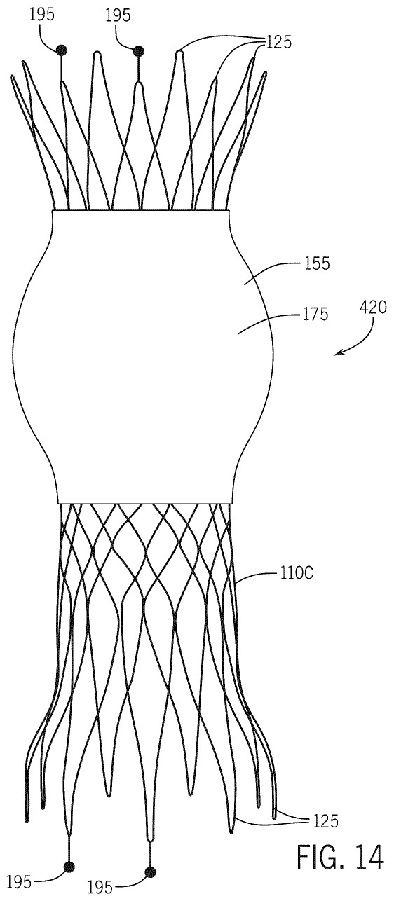

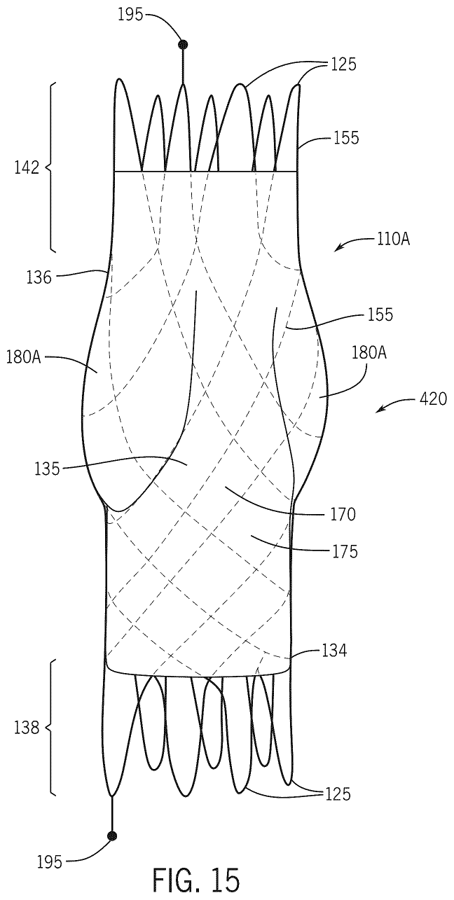

FIG. 12 is a side or elevational view illustrating a representative second embodiment of an outer valve frame 110C in accordance with the disclosure herein. FIG. 13 is a partial, side or elevational view illustrating a representative third embodiment of an outer valve frame 110D with a central coating 175 in accordance with the disclosure herein. FIG. 14 is a side or elevational view illustrating the representative second embodiment of an outer valve frame 110C with a central coating 175 and a plurality of locking tabs 195 in accordance with the disclosure herein. FIG. 15 is a side or elevational view illustrating a representative fourth embodiment of an outer valve frame 110A with two individual sinuses (one sinus per valve leaflet) with a central coating 175 in accordance with the disclosure herein. FIG. 16 is a side or elevational view illustrating a representative fifth embodiment of an outer valve frame 110B with three individual sinuses (one sinus per valve leaflet) with a central coating 175 in accordance with the disclosure herein.

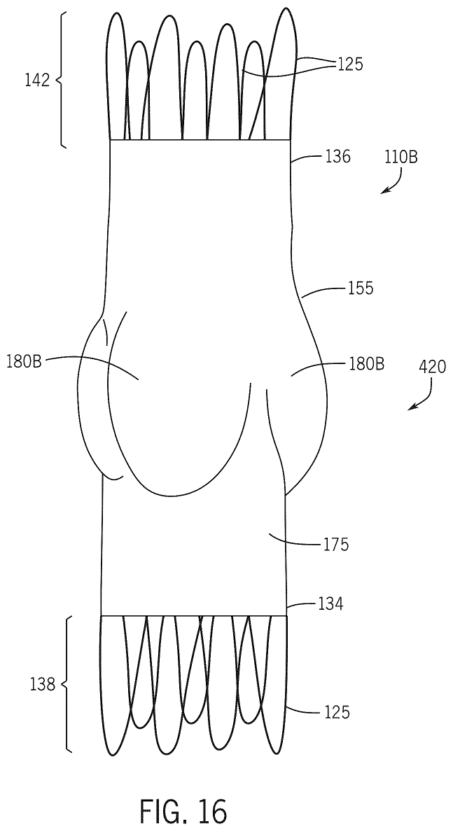

FIG. 17 is a cut-away view illustrating the representative second embodiment of a prosthetic venous valve assembly 100A deployed in situ within a representative vein 182 in accordance with the disclosure herein. It should be noted that portions of the structures of the frames 110A, 110B, 110 in FIGS. 15-17 have been simplified to better illustrate the other structures of these embodiments, such as the various sinuses 180A, 180B and the placement of the prosthetic venous valve assembly 100A deployed in situ.

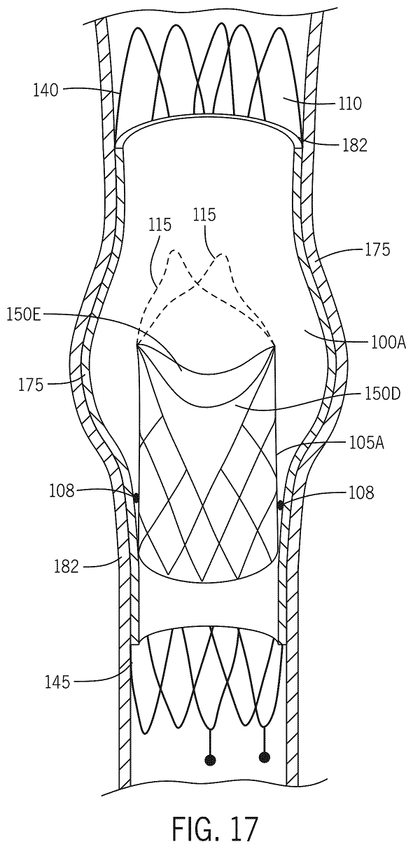

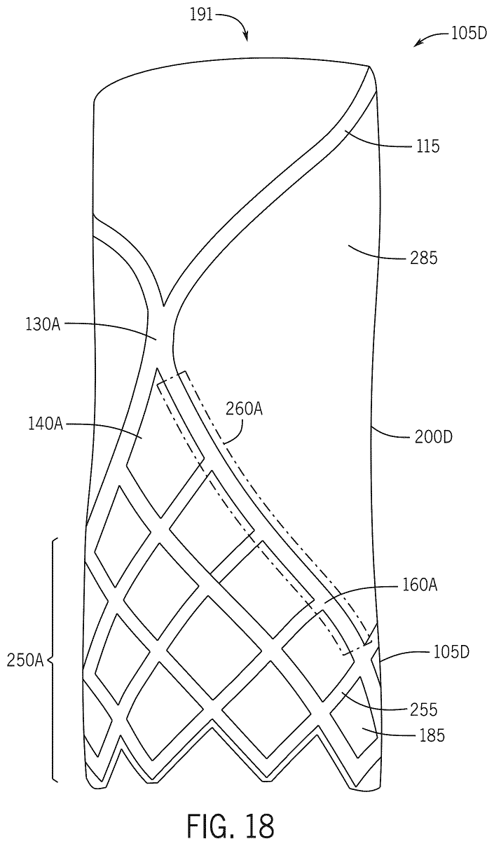

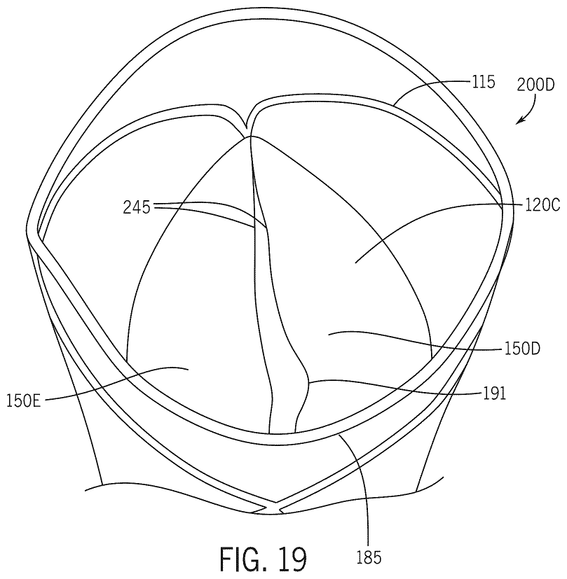

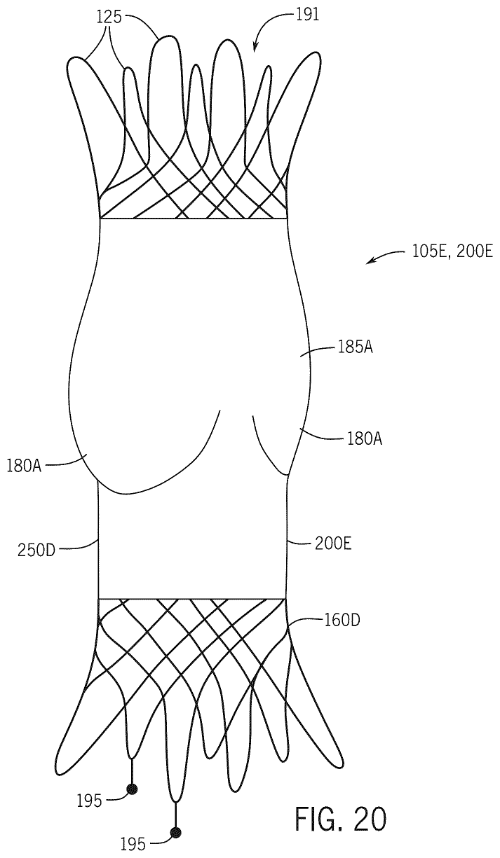

FIG. 18 is a side or elevational view illustrating a representative seventh embodiment of a prosthetic venous valve assembly 200D and also illustrating a representative fifth embodiment of a frame and venous valve assembly 105D in accordance with the disclosure herein. FIG. 19 is a top view illustrating the representative seventh embodiment of a prosthetic venous valve assembly 200D and also illustrating the representative fifth embodiment of a frame and venous valve assembly 105D in accordance with the disclosure herein. FIG. 20 is a side or elevational view illustrating a representative eighth embodiment of a prosthetic venous valve assembly 200E and also illustrating a representative sixth embodiment of a frame and venous valve assembly 105E in accordance with the disclosure herein. FIG. 21 is a cut-away view illustrating the representative eighth embodiment of the prosthetic venous valve assembly 200E and also illustrating the representative sixth embodiment of a frame and venous valve assembly 105E in accordance with the disclosure herein. It should be noted that portions of the structures of the frame 160D in FIGS. 15-17 have been simplified to better illustrate the other structures of this embodiment, such as the venous valve 120E.

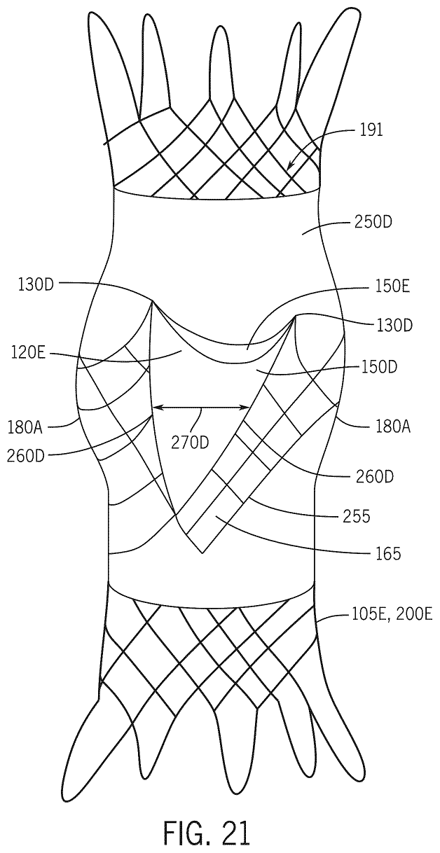

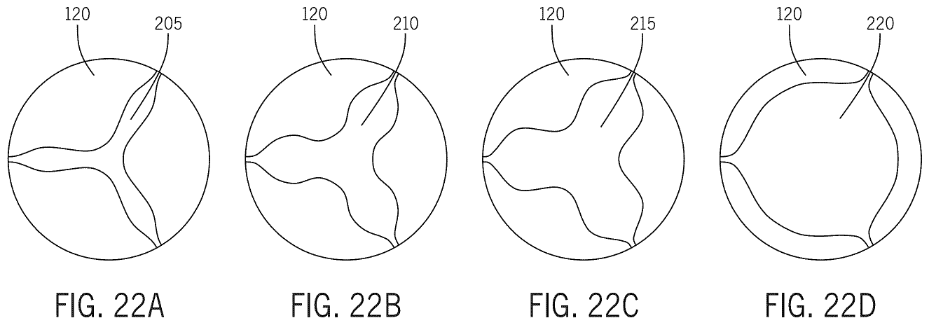

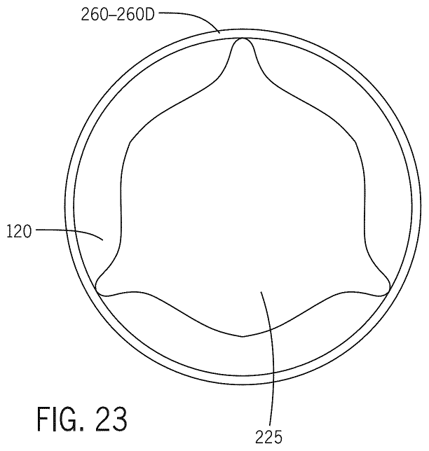

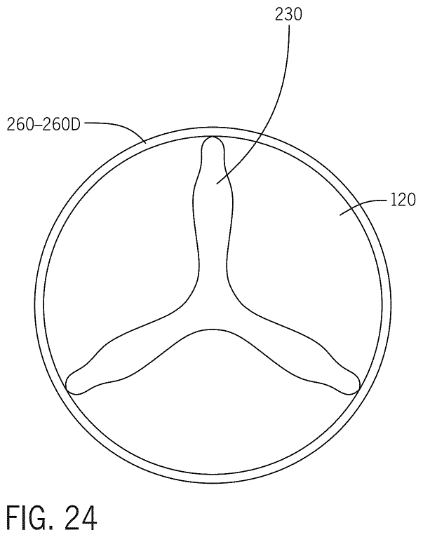

FIGS. 22A, 22B, 22C, and 22D are top or plan (outflow) views respectively illustrating a closed bias 205, a first neutral bias 210, a second neutral bias 215, and an open bias 220 of the representative first embodiment of the prosthetic venous valve 120 in accordance with the disclosure herein. FIG. 23 is a top or plan (outflow) view illustrating an open state (or configuration) 225 of the representative first embodiment of the prosthetic venous valve 120 in accordance with the disclosure herein. FIG. 24 is a top or plan (outflow) view illustrating a partially closed state (or configuration) 230 of the representative first embodiment of the prosthetic venous valve 120 in accordance with the disclosure herein, such as when the valve 120 is in a neutral, unpressurized state. FIG. 25 is a top or plan (outflow) view illustrating a closed state (or configuration) 235 of the representative first embodiment of the prosthetic venous valve 120 in accordance with the disclosure herein.

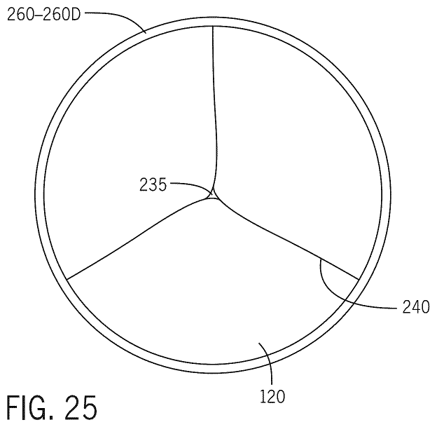

FIGS. 26A, 26B, 26C, 26D, and 26E are cross-sectional schematic illustrations showing blood flow through the opening and closing states of a representative second embodiment of the prosthetic venous valve assembly in accordance with the disclosure herein, with FIG. 26A showing blood flow through a prosthetic venous valve assembly in a first partially open state, FIG. 26B showing blood flow through the prosthetic venous valve assembly in a second partially open state, FIG. 26C showing blood flow through the prosthetic venous valve assembly in a fully open state, FIG. 26D showing blood flow through the prosthetic venous valve assembly in a closing or partially closed state, and FIG. 26E showing no blood flow through the prosthetic venous valve assembly in a fully closed state.

Referring to the FIGS. 1-3, a representative first embodiment of a prosthetic venous valve assembly 100 comprises a first, outer valve frame 110 having an interior, central lumen or channel 190 and a frame and venous valve assembly 105 arranged within the interior, central lumen or channel 190 of the outer valve frame 110. The frame and venous valve assembly 105 is also described in greater detail below with reference to FIGS. 6-8. The frame and venous valve assembly 105 may be abutting and coupled to the outer valve frame 110, such as through welds, rivets, sutures, other ties, possibly cements or adhesives, and so on, illustrated as one or more frame couplings 108. The frame and venous valve assembly 105 comprises a second, inner valve frame 160 and a polymeric, prosthetic venous valve 120 (also referred to as a prosthetic venous valve 120 or more simply as a venous valve 120) coupled to the second, inner valve frame 160, with the prosthetic venous valve 120 having a plurality of valve leaflets 150 (e.g., 150A-150C) (also referred to individually and collectively as a leaflet 150 or leaflets 150). The representative prosthetic venous valve 120 is illustrated as having a tri-leaflet 150 configuration, with three leaflets 150A, 150B, and 150C. Alternatively and equivalently, the prosthetic venous valve assembly 100 may be considered to comprise a first, outer valve frame 110 having an interior, central lumen or channel 190; a second, inner valve frame 160 arranged within the interior, central lumen or channel 190 of the outer valve frame 110; and a polymeric, prosthetic venous valve 120 coupled to the second, inner valve frame 160.

Referring to the FIGS. 4-5, a representative second embodiment of a prosthetic venous valve assembly 100A comprises a first, outer valve frame 110 having an interior, central lumen or channel 190 and a frame and venous valve assembly 105A arranged within the interior, central lumen or channel 190 of the outer valve frame 110. The frame and venous valve assembly 105A also may be coupled (couplings 108) to the outer valve frame 110. The frame and venous valve assembly 105A comprises a second, inner valve frame 160A and a polymeric, prosthetic venous valve 120A (also referred to as a prosthetic venous valve 120A or more simply as a venous valve 120A) coupled to the second, inner valve frame 160A, with the prosthetic venous valve 120A having a plurality of valve leaflets 150 (e.g., 150D-150 E) (also referred to individually and collectively as a leaflet 150 or leaflets 150). The representative prosthetic venous valve 120A is illustrated as having a bi-leaflet 150 configuration, with two leaflets 150D and 150E. Alternatively and equivalently, the prosthetic venous valve assembly 100A may be considered to comprise a first, outer valve frame 110 having an interior, central lumen or channel 190; a second, inner valve frame 160A arranged within the interior, central lumen or channel 190 of the outer valve frame 110; and a polymeric, prosthetic venous valve 120A coupled to the second, inner valve frame 160A. It should also be noted that any of the various prosthetic venous valve assemblies 100, 100A, 200-200F may be implemented to have any number of leaflets, such as one leaflet (not separately illustrated), or the two and three leaflet configurations as illustrated, for example and without limitation.

The representative second embodiment of a prosthetic venous valve assembly 100A differs from the first embodiment of a prosthetic venous valve assembly 100 insofar as the prosthetic venous valve assembly 100A includes a frame and venous valve assembly 105A instead of a frame and venous valve assembly 105, which among other differences, differ in the number and arrangement of valve leaflets 150 and other structures, as described in greater detail below with reference to FIGS. 6-11, and otherwise the prosthetic venous valve assemblies 100, 100A function identically or similarly to each other.

Unless the context clearly indicates to the contrary: (1) reference to any prosthetic venous valve assembly 100, 100A, 200-200F shall be understood to mean and include any other prosthetic venous valve assembly 100, 100A, 200-200F; (2) reference to any frame and venous valve assembly 105-105F shall be understood to mean and include any other frame and venous valve assembly 105-105F; (3) reference to any outer valve frame 110-110D shall be understood to mean and include any other outer valve frame 110-110D; and (4) reference to any prosthetic venous valve 120-120D, shall be understood to mean and include any other prosthetic venous valve 120-120D.

In addition, unless the context clearly indicates to the contrary, any of the various features or elements of a prosthetic venous valve assembly 100, 100A, 200-200F may be applied or included in any of the other prosthetic venous valve assemblies 100, 100A, 200-200F. For example and without limitation, any of the various frame and venous valve assemblies 105-105F may also be utilized with any of the outer valve frames 110-110D.

Referring to the FIGS. 1-5 and 12-17, the outer valve frame 110-110D interfaces with and also provides shape to the vessel wall when inserted into a subject's vein 182, as illustrated in FIG. 17. The outer valve frame 110-110D comprises one or more metal struts 155 forming a central frame matrix 135 (or, equivalently, a central frame member or portion 135) having a first plurality of closed cells 170 and optionally forming one or more sinuses 180, 180A, 180B, with the central frame matrix 135 having a proximal end 136 and a distal end 134. The closed cells 170 are referred to as “closed” because the struts 155 are integrally formed (e.g., laser cut) or otherwise coupled to each other (couplings 112), rather than “open” cells which would be considered to merely abut (e.g., cross) and would be moveable with respect to each other. The closed cells 170 nonetheless have openings (holes or apertures) 114 which may be covered (FIGS. 13-17) or uncovered (FIGS. 1-5, 12). The central frame matrix 135 (and second frame matrix 250) may also be considered to be a mesh, but is referred to as a “matrix” or “member” because in many instances, “mesh” may refer to a braided wire configuration, having moveable wire crossings, rather than solidly or fixedly coupled to or integrally formed (112) with each other (e.g., formed by laser cutting of a tube, for example and without limitation).

In representative embodiments, the outer valve frame 110-110D may include a nickel titanium alloy such as Nitinol, but alternatively may be fabricated from any other suitable material, such as titanium, stainless steel, surgical steel, cobalt chromium (CoCr), another metal, a metallic alloy, carbon fiber, a biocompatible polymer, and combinations thereof, for example and without limitation. The outer valve frame 110-110D remodels the native anatomy to create a sinus 180, 180A, 180B region inside the native vein wall once the outer valve frame 110-110D is fully deployed, as illustrated in FIG. 17. In addition, any pre-existing thrombus in the vein will be moved or trapped between the outer valve frame 110-110D and the epithelium of the vein 182. The optional sinus(es) 180, 180A, 180B may aid in anchoring the outer valve frame 110-110D in the blood vessel and also may aid in blood washout behind the leaflets 150 of the prosthetic venous valve assembly 100, 100A, 200-200F. The outer valve frame 110-110D allows the inner venous valve 120-120D to maintain circularity and expand in vivo to a pre-defined size.

As illustrated in the various Figures, the one or more optional sinuses 180, 180A, 180B may have a spherical (360 degree) shape such as sinus 180 or bulbous shape such as sinuses 180A, 180B, and the outer valve frame 110-110D may also have a distal cylindrical portion 144 as illustrated. As described in greater detail below, the valve leaflets 150 are arranged within and moveable within the central frame matrix 135 and/or sinuses 180, 180A, 180B, with the one or more sinuses 180, 180A, 180B acting to aid blood circulation out of the prosthetic venous valve 120 and avoid thrombus formation. The one or more sinuses 180, 180A, 180B may also expand and contract with blood flow through the vein, also assisting with blood washout and valve leaflet 150A-150G opening and closing. As illustrated in FIGS. 13-17, the outer valve frame 110-110D may also include a polymeric coating 175 which covers or surrounds the central frame matrix 135 (and potentially other portions of the outer valve frame 110-110D as well), which also serves to prevent thrombus formation, provides a controlled environment which prevents tissue ingrowth, prevents paravalvular leakage, and potentially assists blood washout and valve leaflet 150 kinematics. The sinus(es) 180, 180A, 180B also serve to anchor the prosthetic venous valve assembly 100, 100A, 200-200F within the vein once deployed. As illustrated, blood flows in the direction 132 (indicated by the arrow 132) through the prosthetic venous valve assembly 100, 100A, 200-200F, through the interior, central lumen or channel 190 of the outer valve frame 110-110D and the prosthetic venous valve 120-120D. A prosthetic venous valve assembly 200D, which does not include any sinus 180, is illustrated and described below with reference to FIGS. 18 and 19.

As illustrated in FIG. 15, instead of a single, unitary spherically-shaped sinus 180 open for movement of all of the various valve leaflets 150, the outer valve frame 110A has two bulbous-shaped sinuses 180A. In this embodiment, the outer valve frame 110A may be included with a prosthetic venous valve 120 having a bi-leaflet configuration (e.g., prosthetic venous valve 120A, 120D), with each valve leaflet 150D, 150E moveable in a corresponding sinus 180A. As illustrated in FIG. 16, also instead of a single, unitary spherically-shaped sinus 180 open for movement of all of the various valve leaflets 150, the outer valve frame 110B has three bulbous-shaped sinuses 180B. In this embodiment, the outer valve frame 110B may be included with a prosthetic venous valve 120 having a tri-leaflet configuration (e.g., prosthetic venous valve 120), with each valve leaflet 150A, 150B, 150C moveable in a corresponding sinus 180B.

In various representative embodiments, a plurality of adaptive, closed cell strut extension members 125 extend distally and proximally from the central frame matrix 135 of the outer valve frame 110-110C. More particularly, a first plurality 138 of adaptive, closed cell strut extension members 125 are integrally formed with and extend distally from the distal end 134 of the central frame matrix 135, and a second plurality 142 of adaptive, closed cell strut extension members 125 are integrally formed with and extend proximally from the proximal end 136 of the central frame matrix 135. As illustrated, the plurality of adaptive, closed cell strut extension members 125 are generally triangular in shape, inverted U-shaped, or otherwise smoothly curved in shape and each adaptive, closed cell strut extension member 125 has a free end 192, 194. As a result, the adaptive, closed cell strut extension members 125 provide inflow and outflow adaptability and flexibility to the outer valve frame 110-110C and can conform to and move with the native blood vessel as the vessel may dilate or constrict. In addition, the adaptive, closed cell strut extension members 125 are generally uncovered, allowing for neointimal ingrowth of epithelium into the openings (holes or apertures) 114 of the adaptive, closed cell strut extension members 125, anchoring of the prosthetic venous valve assembly 100, 100A, 200-200F within the subject vein, and avoiding restriction of inflow and outflow through the prosthetic venous valve assembly 100, 100A, 200-200F and subject vein. The adaptive, closed cell strut extension members 125 also act as a chronic seal to prevent any perivalvular leak and may support in anchoring and migration resistance, and may facilitate tissue ingrowth for chronic anchoring and sealing. Not separately illustrated, the outer valve frame 110-110C inflow side may also be covered with a polymer, serving as a skirt to prevent paravalvular leakage.

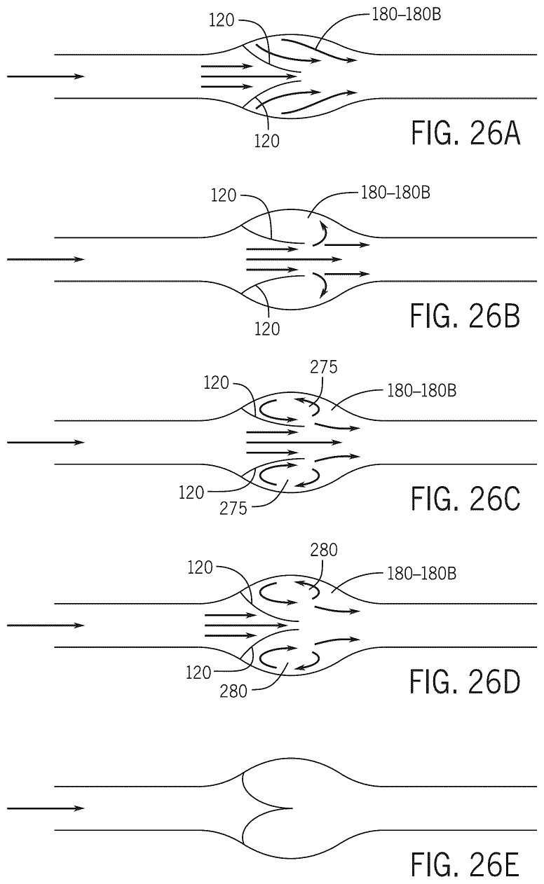

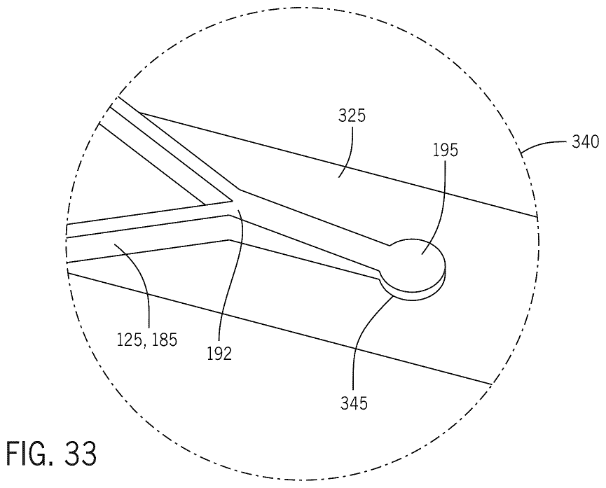

Also as illustrated in the various Figures, the outer valve frame 110-110D may also include one or more locking tabs 195 extending from one or more of the pluralities of adaptive, closed cell strut extension members 125, distally, proximally, or both distally and proximally. The one or more locking tabs 195 have a size and configuration structured, adapted or configured to be removably insertable into one or more corresponding or mating keys or slots 345 of a delivery catheter 300, 300A. When a prosthetic venous valve assembly 100, 100A, 200-200F is loaded onto a delivery catheter 300, 300A, the one or more locking tabs 195 are inserted into the corresponding or mating keys or slots 345, and the prosthetic venous valve assembly 100, 100A, 200-200F may be compressed, collapsed or crimped to a smaller diameter for insertion into the subject vein. Once inserted at its desired or selected location, the prosthetic venous valve assembly 100, 100A, 200-200F may be expanded (e.g., self-expanded or balloon expanded) to its expanded or deployed state and, as it is expanded, the one or more locking tabs 195 pull out of the corresponding or mating keys or slots 345 and the prosthetic venous valve assembly 100, 100A, 200-200F is thereby released from the delivery catheter 300, 300A, such as illustrated in FIGS. 34-36. Alternatively, the prosthetic venous valve assembly 100, 100A, 200-200F may be deployed in two stages, first deploying the outer valve frame 110-110D, which then may serve as a dock within the vein, followed by deploying the frame and venous valve assembly 105-105F into the lumen 190 of the outer valve frame 110-110D. In the latter case, the frame and venous valve assembly 105-105F may also be provided with one or more locking tabs 195, not separately illustrated. In addition, the various prosthetic venous valve assemblies 100, 100A, 200-200F and their components may be provided with radiopaque markers, such as to provide appropriate axial and/or longitudinal alignment during deployment. The prosthetic venous valve assemblies 100, 100A, 200-200F are illustrated in their respective fully expanded state 420 in FIGS. 1-21, and in their respective crimped or compressed state (or configuration) 405 in FIGS. 27-31 and partially crimped or compressed in FIGS. 34-36.

As mentioned above, in various representative embodiments, a frame and venous valve assembly 105-105F is arranged within the lumen 190 of the first, outer valve frame 110, 110A, 110B, and is allowed to fully expand (or partially expand, depending upon the vessel stiffness), and may be self-expanding or balloon expandable. In other representative embodiments, a frame and venous valve assembly 105-105F may be deployed directly as a prosthetic venous valve assembly 200-200F, without an outer valve frame 110-110D, and is also allowed to fully expand, and may be self-expanding or balloon expandable. As described in greater detail below, the various embodiments of a frame and venous valve assembly 105-105F primarily differ from each other with regard to the number and arrangement of valve leaflets 150, the inclusion of optional commissure support strut (or wire) halos 115, and the type of polymer utilized to form the prosthetic venous valve 120-120D.

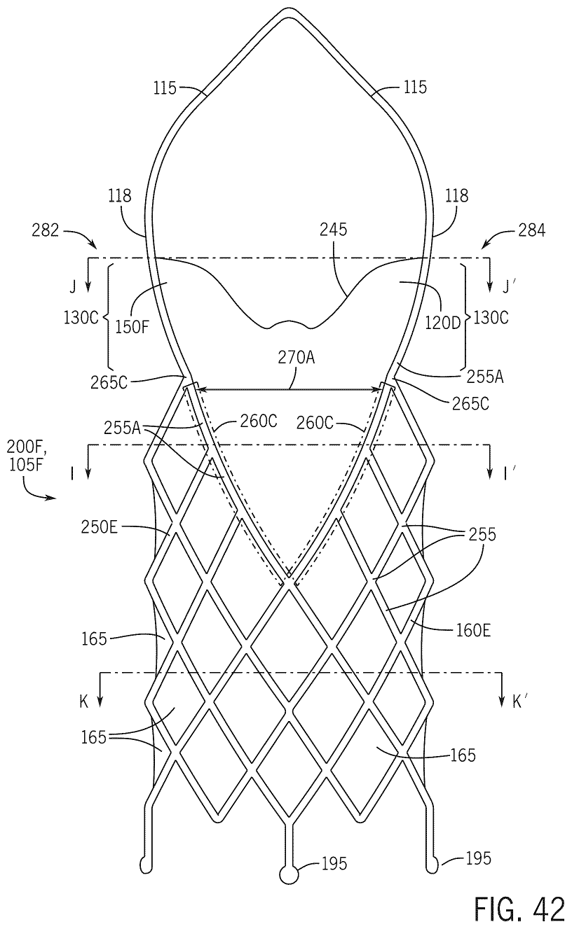

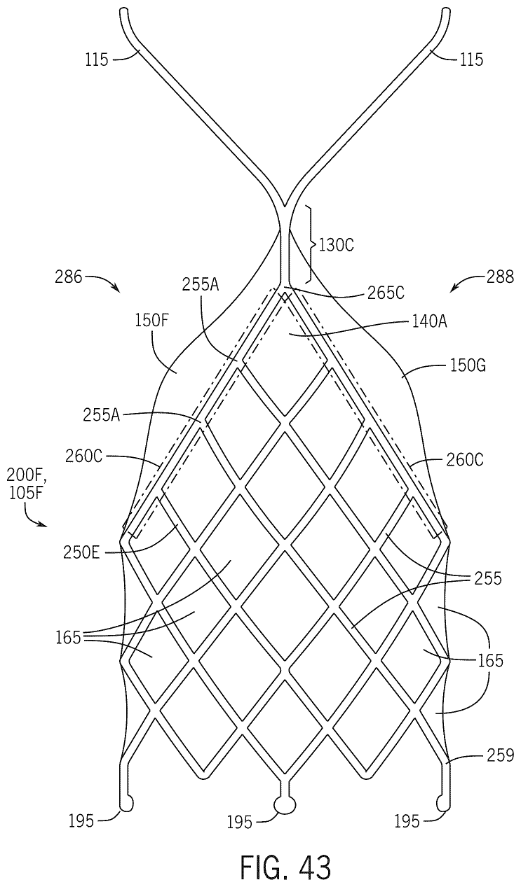

Referring to FIGS. 6-8, a representative first embodiment of a frame and venous valve assembly 105 and/or a prosthetic venous valve assembly 200 comprises a second, (inner) valve frame 160 having an (inner) valve lumen 191 and a polymeric, prosthetic venous valve 120 moveable in or otherwise arranged in the lumen 191, with the prosthetic venous valve 120 (referred to equivalently as a polymeric venous valve 120) having an open state 225 and a partially closed state 230 or a fully closed state 235. The second, inner valve frame 160 comprises one or more metal struts 255 forming a second frame matrix 250, with the second frame matrix 250 having a second plurality of closed cells 165 and also forming a plurality of valve commissure supports 130 and a plurality of lateral valve supports 260. The second frame matrix 250 is also arranged to form a plurality of inverted “V” (or triangular) shapes or inverted “U” shapes, providing openings or gaps 270 in between the plurality of lateral valve supports 260. The proximal ends of respective lateral valve supports 260 meet at an apex 265 of the respective triangular or U-shaped regions 140 to form a corresponding valve commissure support 130. In other embodiments, the valve commissure supports 130 may be further elongated, such as illustrated in FIGS. 42 and 43. Additionally, any of the various inner valve frames 160 may also comprise adaptable inflow and outflow sections, a relatively stiffer valve coupling section, and a valve sinus section, as described in greater detail below. Not separately illustrated in FIG. 6, the valve frame 160 also may also include a plurality of adaptive, closed cell strut extension members 125 extending distally (inflow end), particularly when implemented to be a prosthetic venous valve assembly 200 without any outer frame 110-110D.

The various components forming a transcatheter, prosthetic venous valve assembly 200-200F may be described multiple ways. From a first point of view, a transcatheter, prosthetic venous valve assembly 200-200F comprises a valve frame (160-160E) comprising a plurality of struts 255 forming a frame matrix 250-250E having a plurality of closed cells 165 and a valve frame lumen 191, with the plurality of struts 255 further forming a plurality of lateral valve supports (260-260D) and a plurality of valve commissure supports (130-130C); a polymeric frame coating 185 encapsulating the frame matrix 250-250E; and a polymeric venous valve 120-120D having an open or partially open state and a closed or partially closed state, the polymeric venous valve arranged in the lumen, with the polymeric venous valve 120-120D comprising: one or more polymeric leaflets 150 integrally formed with or coupled to the polymeric frame coating 185, with each leaflet 150 of the one or more polymeric leaflets 150 moveable between the open (or partially open) state 225 and the closed or partially closed state 235, 230. From a second point of view, a transcatheter, prosthetic venous valve assembly 200-200F comprises a valve frame (160-160E) comprising a plurality of struts 255 forming a frame matrix 250-250E having a plurality of closed cells 165 and a valve frame lumen 191, with the plurality of struts 255 further forming a plurality of lateral valve supports (260-260D) and a plurality of valve commissure supports (130-130C); and a prosthetic (or polymeric) venous valve 120-120D having an open or partially open state and a closed or partially closed state, the polymeric venous valve arranged in the lumen, with the polymeric venous valve 120-120D comprising: a polymeric frame coating 185 encapsulating the frame matrix 250-250E; and one or more polymeric leaflets 150 integrally formed with or coupled to the polymeric frame coating 185, with each leaflet 150 of the one or more polymeric leaflets 150 moveable between the open (or partially open) state 225 and the closed or partially closed state 235, 230. Any and all of these descriptions and categories of components are considered equivalent and within the scope of the disclosure.

In greater detail, the polymeric, prosthetic venous valve 120 comprises an inner frame coating 185 conformally encapsulating, coupled to or otherwise surrounding the second frame matrix 250, including a plurality of lateral valve supports (260-260C) and a plurality of valve commissure supports (130-130C); and a plurality of valve leaflets 150, illustrated as valve leaflets 150A 150B, and 150C, which are integrally formed with the inner frame coating 185, with each leaflet 150 of the plurality of leaflets 150 moveable to form the partially closed state 230 or the fully closed state 235. The inner frame coating 185 of the polymeric, prosthetic venous valve 120 comprises a polymeric layer conformally encapsulating, adhered to or surrounding the second, inner valve frame 160 and second frame matrix 250, may have a thickness between 40-60 microns, or between 45-55 microns, or about 50 microns, for example and without limitation, with additional dimensions described in greater detail below. The inner frame coating 185 of the polymeric, prosthetic venous valve 120 coating the frame matrix 250 and forming the leaflets 150 thereby provides a sutureless attachment of the leaflets 150 to the frame matrix 250. Depending on the angle of cutting, the laser cut struts 255 (and 155) may have a single bevel and may be trapezoidal in cross-section, as illustrated in FIGS. 7 and 8, with additional cross-sectional shapes described in greater detail below, such as hexagonal with a double-bevel. While referred to as an inner frame coating 185, the frame coating 185 is “inner” only insofar as it encapsulates the inner valve frame 160 and frame matrix 250. Also as illustrated in FIG. 7, the frame coating 185 conformally coats or encapsulates both an inner portion 156 and an outer portion 158 of the valve frame 160 and frame matrix 250. Also as illustrated in FIG. 6, the polymer forming the inner frame coating 185 may be sufficiently thin or translucent, such that while the inner frame coating 185 substantially coats and surrounds the second frame matrix 250, the second frame matrix 250 may nonetheless also be visible through the frame coating 185.

The prosthetic venous valve 120 is illustrated as a tri-leaflet venous valve, having three valve leaflets 150A, 150B, and 150C, which are also integrally formed as a polymeric layer or sheet with and extending from the inner frame coating 185 within the respective openings or gaps 270 defined by the lateral valve supports 260 of the second frame matrix 250. The valve leaflets 150A, 150B, and 150C are each flexible and moveable within the respective opening or gap 270, and generally will move correspondingly to the pressure exerted by fluid, such as blood, flowing through the prosthetic venous valve 120. Each valve leaflet 150A, 150B, and 150C terminates in a respective valve edge 245 and at the lateral valve supports 260. In a (fully) closed state, the respective valve edges 245 meet with and abut one or more valve edges 245 of adjacent valve leaflets 150 to form a corresponding commissure 240 in a fully closed state, as illustrated in FIG. 25. In various representative embodiments, the valve leaflets 150A, 150B, and 150C are formed, structured, configured, or shaped during fabrication to have sinusoidal, scalloped or straight valve edges 245, for example and without limitation, and may be fabricated to provide varying degrees of valve closure, such as fully or partially closed.

Not separately illustrated, as an option, the valve leaflets 150A-150G may also be structurally reinforced with nitinol or other metallic or carbon fiber wires or films during fabrication, such as through having nitinol wires formed in a valve leaflet 150A-150G geometry and dipping them in a polymer to form the valve leaflets 150A-150G, as described in greater detail below. Also not separately illustrated, also as an option, nitinol wire springs can also be implemented to assist in the opening and closing of the valve leaflets 150A-150G. A nitinol film may also be utilized in forming the sinus(es) 180, 180A, 180B, also as an option.

Referring to FIG. 9, a representative third embodiment of a frame and venous valve assembly 105B and/or a prosthetic venous valve assembly 200B comprises a second, inner valve frame 160B having an (inner) valve lumen 191 and a polymeric, prosthetic venous valve 120A arranged in the lumen 191 and the prosthetic venous valve 120A having an open state 225 and a partially closed state 230 or a fully closed state 235. The second, inner valve frame 160B comprises one or more metal struts 255 forming a second frame matrix 250B, with the second frame matrix 250B having a second plurality of closed cells 165 and also forming a plurality of valve commissure supports 130A and a plurality of lateral valve supports 260A. The second frame matrix 250B is also arranged to form a plurality of more arcuate or curvilinear inverted “V”, inverted “U”, or triangular shapes, providing openings or gaps 270A in between the plurality of lateral valve supports 260A. The proximal ends of respective lateral valve supports 260A meet at an apex 265A of the respective diamond (triangular) regions 140A to form a corresponding valve commissure support 130A. Not separately illustrated in FIG. 9, the valve frame 160B also may also include a plurality of adaptive, closed cell strut extension members 125 extending distally (inflow end), particularly when implemented to be a prosthetic venous valve assembly 200B without any outer frame 110-110D.

Referring to FIGS. 4-5, a representative second embodiment of a frame and venous valve assembly 105A and/or a prosthetic venous valve assembly 200A also comprises a second, inner valve frame 160A having an (inner) valve lumen 191 and a polymeric, prosthetic venous valve 120A arranged or moveable in the lumen 191, with the prosthetic venous valve 120A having an open state 225 and a partially closed state 230 or a fully closed state 235. The second, inner valve frame 160A also comprises one or more metal struts 255 forming a second frame matrix 250A, with the second frame matrix 250A having a second plurality of closed cells 165 and also forming a plurality of valve commissure supports 130 and a plurality of lateral valve supports 260A. The second frame matrix 250A is also arranged to form a plurality of more arcuate or curvilinear inverted “V”, inverted “U”, or triangular shapes, providing openings or gaps 270A in between the plurality of lateral valve supports 260A. The proximal ends of respective lateral valve supports 260A meet at an apex 265A of the respective diamond (triangular) regions 140A to form a corresponding valve commissure support 130. Not separately illustrated in FIGS. 4-5, the valve frame 160A also may also include a plurality of adaptive, closed cell strut extension members 125 extending distally (inflow end), particularly when implemented to be a prosthetic venous valve assembly 200A without any outer frame 110-110D.

The frame and venous valve assembly 105A only includes a few differences from the frame and venous valve assembly 105B. First, the second frame matrix 250A is more cylindrical than the second frame matrix 250B, which curves inward proximally. Second, the frame and venous valve assembly 105B includes a plurality of commissure support strut (or wire) halos 115, which are considered optional for the frame and venous valve assembly 105A (and indicated only in dashed lines). Third, the angle “a” 148A is more acute for the frame and venous valve assembly 105B, such that the valve leaflets 150D, 150E extend more proximally with the plurality of lateral valve supports 260A in the frame and venous valve assembly 105B. Otherwise, the structures and functions of the frame and venous valve assembly 105A and frame and venous valve assembly 105B are nearly identical, and will be described in greater detail below with reference to the frame and venous valve assembly 105B, understanding that the features described for the frame and venous valve assembly 105B are equally applicable to and may be incorporated within the frame and venous valve assembly 105A.

Referring again to FIG. 9, the polymeric, prosthetic venous valve 120A comprises an inner frame coating 185 coupled to or surrounding the second frame matrix 250B; and a plurality of valve leaflets 150, illustrated as valve leaflets 150D and 150E, which are integrally formed with the inner frame coating 185, with each leaflet of the plurality of leaflets moveable to form the partially closed state 230 or the fully closed state 235. The inner frame coating 185 of the polymeric, prosthetic venous valve 120A also comprises a polymeric layer conformally encapsulating, adhered to or surrounding the second, inner valve frame 160B and second frame matrix 250B. As illustrated in FIG. 9, the polymer forming the inner frame coating 185 also may be sufficiently thin or translucent, such that while the inner frame coating 185 substantially coats and surrounds the second frame matrix 250B, the second frame matrix 250B may nonetheless be visible through the inner frame coating 185, as illustrated.

The prosthetic venous valve 120A is illustrated as a bi-leaflet venous valve, having two valve leaflets 150D and 150E, which are also integrally formed as a polymeric layer or sheet with and extend from the inner frame coating 185 within the respective openings or gaps 270A defined by the lateral valve supports 260A of the second frame matrix 250B. The valve leaflets 150D and 150E also are each flexible and moveable within the respective opening or gap 270A, and generally will move correspondingly to the pressure exerted by fluid, such as blood, flowing through the prosthetic venous valve 120A. Each valve leaflet 150D and 150E also terminates in a respective valve edge 245 and at the lateral valve supports 260A. In a closed state, the respective valve edges 245 meet with and abut one or more valve edges 245 of adjacent valve leaflets 150 to form a corresponding commissure 240 in a fully closed state, similarly to what is illustrated in FIG. 25 for the tri-leaflet valve. In various representative embodiments, the valve leaflets 150D and 150E are also formed, structured, configured, or shaped during fabrication to have sinusoidal or scalloped valve edges 245, for example and without limitation, and may be fabricated to provide varying degrees of valve closure, such as fully or partially closed.

The frame and venous valve assembly 105B differs from the frame and venous valve assembly 105 in several ways, in addition to having a bi-leaflet rather than tri-leaflet configuration. The second, inner valve frame 160B of the frame and venous valve assembly 105B further comprises, as an option, a plurality of commissure support strut (or wire) halos 115 (or, equivalently referred to as commissure support struts 115), with each commissure support strut halo 115 coupled between corresponding valve commissure supports 130A, such that each end 118 of each commissure support strut halo 115 is coupled to and extending proximally from a corresponding valve commissure support 130A, as illustrated. The commissure support strut halos 115 may be included when the frame and venous valve assembly 105B is to be deployed directly as a prosthetic venous valve assembly 200B without any outer valve frame 110-110D. While illustrated as “wishbone” shaped, the commissure support strut halos 115 may have any suitable arcuate configuration or shape, and serve to stiffen and stabilize the corresponding valve commissure supports 130A and prevent excessive inward (distal) deflection of the valve leaflets 150D and 150E, as well as maintain the roundness of the valve 120A. The commissure support strut halos 115 may be integrally fabricated with the rest of the second, inner valve frame 160B. The frame and venous valve assembly 105B also illustrates a second, inner valve frame 160B in which the angle “α” 148A, described in greater detail below, is significantly more acute than the angle “α” 148 of the frame and venous valve assembly 105, providing a more elongated diamond or triangular-shaped region 140A. The frame and venous valve assembly 105B also illustrates more arcuate or curvilinear lateral valve supports 260A and inverted “V”, inverted “U”, or triangular shapes of the second frame matrix 250B, providing openings or gaps 270A in between the plurality of lateral valve supports 260A.

In a representative embodiment, the shape of the lateral valve supports 260, 260A (stent boundary) along the leaflet (i.e. the margin of attachment) follows a desired shape to best support the leaflet 150 to minimize strain when the leaflet 150 is in the closed position and supporting the backpressure of the blood. For example and without limitation, this may be a curvilinear shape, and it may be different for a bi-leaflet valve versus a tri-leaflet design. Depending on the design, however, a more linear shape such as the inverted V that is described may be an option for a short distance below the commissure attachment point and this angle “α” 148 will be more acute for a bi-leaflet than a tri-leaflet configuration, also as described in greater detail below.

Referring to FIG. 10, a representative fourth embodiment of a frame and venous valve assembly 105C and/or a prosthetic venous valve assembly 200C comprises a second, inner valve frame 160C having an (inner) valve lumen 191 and a polymeric, prosthetic venous valve 120B arranged in the lumen 191 and the prosthetic venous valve 120B having an open state 225 and a partially closed state 230 or a fully closed state 235. The frame and venous valve assembly 105C and/or a prosthetic venous valve assembly 200C primarily differs from the frame and venous valve assembly 105B and/or a prosthetic venous valve assembly 200B in having a tri-leaflet configuration rather than a bi-leaflet configuration. The second, inner valve frame 160C comprises one or more metal struts 255 forming a second frame matrix 250C, with the second frame matrix 250C having a second plurality of closed cells 165 and also forming a plurality of valve commissure supports 130B and a plurality of lateral valve supports 260B. The second frame matrix 250C is also arranged to form a plurality of more arcuate or curvilinear inverted “V” inverted “U”, or triangular shapes, providing openings or gaps 270B in between the plurality of arcuate or curvilinear lateral valve supports 260B. The proximal ends of respective lateral valve supports 260B also meet at an apex 265B of the respective diamond-shaped (or triangular) regions 140B to form a corresponding valve commissure support 130B. Not separately illustrated in FIG. 10, the valve frame 160C also may also include a plurality of adaptive, closed cell strut extension members 125 extending distally (inflow end), particularly when implemented to be a prosthetic venous valve assembly 200C without any outer frame 110-110D.

The polymeric, prosthetic venous valve 120B of the frame and venous valve assembly 105C and/or a prosthetic venous valve assembly 200C comprises an inner frame coating 185 coupled to or surrounding the second frame matrix 250C; and a plurality of valve leaflets 150, illustrated as valve leaflets 150A 150B, and 150C, which are integrally formed with the inner frame coating 185, with each leaflet of the plurality of leaflets moveable to form the partially closed state 230 or the fully closed state 235. The inner frame coating 185 of the polymeric, prosthetic venous valve 120B also comprises a polymeric layer conformally encapsulating, adhered to or surrounding the second, inner valve frame 160C and second frame matrix 250C. As illustrated in FIG. 10, the polymer forming the inner frame coating 185 is also sufficiently thin or translucent, such that while the inner frame coating 185 substantially coats and surrounds the second frame matrix 250C, the second frame matrix 250C may nonetheless be visible through the inner frame coating 185, as illustrated.

The prosthetic venous valve 120B of the frame and venous valve assembly 105C and/or a prosthetic venous valve assembly 200C is also illustrated as a tri-leaflet venous valve, having three valve leaflets 150A, 150B, and 150C, which are also integrally formed as a polymeric layer or sheet with and extend from the inner frame coating 185 within the respective openings or gaps 270B defined by the lateral valve supports 260B of the second frame matrix 250C. The valve leaflets 150A, 150B, and 150C are each flexible and moveable within the respective opening or gap 270B, and generally also will move correspondingly to the pressure exerted by fluid, such as blood, flowing through the prosthetic venous valve 120B. Each valve leaflet 150A, 150B, and 150C terminates in a respective valve edge 245. In a closed state, the respective valve edges 245 meet with and abut one or more valve edges 245 of adjacent valve leaflets 150 to form a corresponding commissure 240 in a fully closed state, as illustrated in FIG. 25. In various representative embodiments, the valve leaflets 150A, 150B, and 150C are formed, structured, configured, or shaped during fabrication to have sinusoidal or scalloped valve edges 245, for example and without limitation, and may be fabricated to provide varying degrees of valve closure, such as fully or partially closed.

The frame and venous valve assembly 105C differs from the frame and venous valve assembly 105B insofar as the frame and venous valve assembly 105C has a tri-leaflet configuration rather than having a bi-leaflet configuration, with a corresponding number of commissure support strut halos 115, and is otherwise identical. Like the frame and venous valve assembly 105B, the frame and venous valve assembly 105C also differs from the frame and venous valve assembly 105 in several ways. The second, inner valve frame 160C of the frame and venous valve assembly 105C further comprises a plurality of commissure support strut halos 115 (or, equivalently referred to as commissure support struts 115), with each commissure support strut halo 115 also coupled between corresponding valve commissure supports 130B, such that each end 118 of each commissure support strut halo 115 is coupled to and extending proximally from a corresponding valve commissure support 130B, as illustrated. Also while illustrated as “wishbone” shaped, the commissure support strut halos 115 may have any suitable arcuate configuration or shape, and serve to stiffen and stabilize the corresponding valve commissure supports 130B and prevent excessive inward (distal) deflection of the valve leaflets 150A, 150B, and 150C. The commissure support strut halos 115 may be integrally fabricated with the rest of the second, inner valve frame 160C. The commissure support strut halos 115 may be included when the frame and venous valve assembly 105C is to be deployed directly as a prosthetic venous valve assembly 200C without any outer valve frame 110-110D.

Referring to FIG. 11, the representative fourth embodiment of a frame and venous valve assembly 105C differs from that illustrated in FIG. 10 only insofar as the polymer forming the inner frame coating 185 of the polymeric, prosthetic venous valve 120B is sufficiently thick or not translucent, such that the second frame matrix 250C (illustrated with dashed lines) is not visible through the inner frame coating 185, and is otherwise identical.

Those having skill in the art will recognize that a one-part, single frame prosthetic venous valve assembly 200-200F may also be fabricated and deployed in accordance with the disclosure herein. For this prosthetic venous valve assembly 200-200F, use of an outer valve frame 110-110D potentially may be optional.

Referring to FIGS. 18 and 19, for this embodiment, the single frame prosthetic venous valve assembly 200D is illustrated as a bi-leaflet valve 120C and also includes a conduit 285 (having an (inner) valve lumen 191), integrally formed with both the valve leaflets 150D, 150E and part of the inner frame coating 185, such as by using an appropriately shaped, structured, and/or configured mandrel dipped or coated with a polymer. The formed conduit 285 having the valve leaflets 150D, 150E and part of the inner frame coating 185 is subsequently attached or otherwise coupled to the inner valve frame 160A, such as by inserting the formed conduit 285 having the valve leaflets 150D, 150E and part of the inner frame coating 185 into the interior of the inner valve frame 160A and using a syringe application of the polymer or an adhesive to the exterior of the inner valve frame 160A to adhere or bond the formed conduit 285 having the valve leaflets 150D, 150E and part of the inner frame coating 185 to the inner valve frame 160A, followed by curing the polymer, also for example and without limitation. The additional polymer or adhesive utilized to adhere or bond the formed conduit 285 having the valve leaflets 150D, 150E and part of the inner frame coating 185 to the inner valve frame 160A then completes the formation of the inner frame coating 185, which then surrounds the inner valve frame 160A. The prosthetic venous valve assembly 200D may also be viewed as comprising the frame and venous valve assembly 105A with an extended frame coating 185 to form the conduit 285. Not separately illustrate, the conduit 285 may also be shaped and sized to provide one or more sinus(es) 180, 180A, 180B, as shown in FIGS. 20 and 21. The prosthetic venous valve assembly 200C functions similarly to the bi-leaflet prosthetic venous valve assemblies 200A, 200B described above. In addition, not separately illustrated, the prosthetic venous valve assembly 200D may be fabricated to have a tri-leaflet configuration.

Referring to FIGS. 20 and 21, in a representative eighth embodiment of a prosthetic venous valve assembly 200E and a representative sixth embodiment of a frame and venous valve assembly 105E, the single frame 160D is implemented or configured similarly to the frame 110A, having two sinuses 180A, with a bi-leaflet valve 120A configuration of leaflets 150D, 150E, and having many of the other features described above with reference to a frame 110, 110A, such as having a plurality of adaptive, closed cell strut extension members 125. To illustrate the sinuses 180A and the arrangement of the prosthetic venous valve 120E without generating visual confusion, many of the struts 255 forming the frame matrix 250D are not separately illustrated. The prosthetic venous valve assembly 200E comprises a valve frame 160D having an (inner) valve lumen 191 and a polymeric, prosthetic venous valve 120E arranged in the lumen 191 and the prosthetic venous valve 120E having an open state 225 and a partially closed state 230 or a fully closed state 235. The single frame 160D also interfaces with and also provides shape to the vessel wall when inserted into a subject's vein 182. The single frame 160D comprises one or more metal struts 255 forming a frame matrix 250D having a plurality of closed cells 165 forming sinuses 180A, with a plurality of adaptive, closed cell strut extension members 125 extending distally and proximally from the frame matrix 250D, as described above. The valve frame 160D also forms a plurality of valve commissure supports 130D and a plurality of lateral valve supports 260D. The valve frame 160D also forms a plurality of more arcuate or curvilinear inverted “V”, inverted “U”, or triangular shapes, providing openings or gaps 270D in between the plurality of lateral valve supports 260D.

The prosthetic venous valve assembly 200E comprises a frame coating 185A coupled to or surrounding the central portion of the frame matrix 250D; and a plurality of valve leaflets 150, illustrated as valve leaflets 150D and 150E, which are integrally formed with the frame coating 185A, with each leaflet of the plurality of leaflets moveable to form the partially closed state 230 or the fully closed state 235. The prosthetic venous valve assembly 200E is illustrated as having a bi-leaflet venous valve, having two valve leaflets 150D and 150E, which are also integrally formed as a polymeric layer or sheet with and extend from the frame coating 185A within the respective openings or gaps 270D defined by the lateral valve supports 260D of the frame matrix 250D. The valve leaflets 150D and 150E also are each flexible and moveable within the respective opening or gap 270D, and generally will move correspondingly to the pressure exerted by fluid, such as blood, flowing through the prosthetic venous valve assembly 200E. Each valve leaflet 150D and 150E also terminates in a respective valve edge 245. In a closed state, the respective valve edges 245 meet with and abut one or more valve edges 245 of adjacent valve leaflets 150 to form a corresponding commissure 240 in a fully closed state, similarly to what is illustrated in FIG. 25 for the tri-leaflet valve. In various representative embodiments, the valve leaflets 150D and 150E are also formed, structured, configured, or shaped during fabrication to have sinusoidal or scalloped valve edges 245, for example and without limitation, and may be fabricated to provide varying degrees of valve closure, such as fully or partially closed.

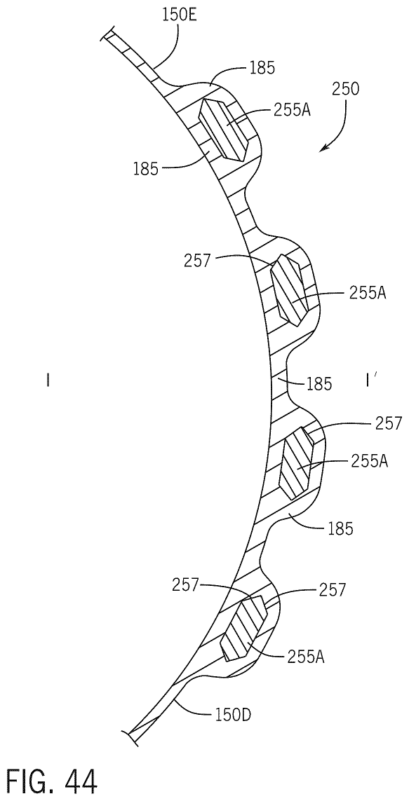

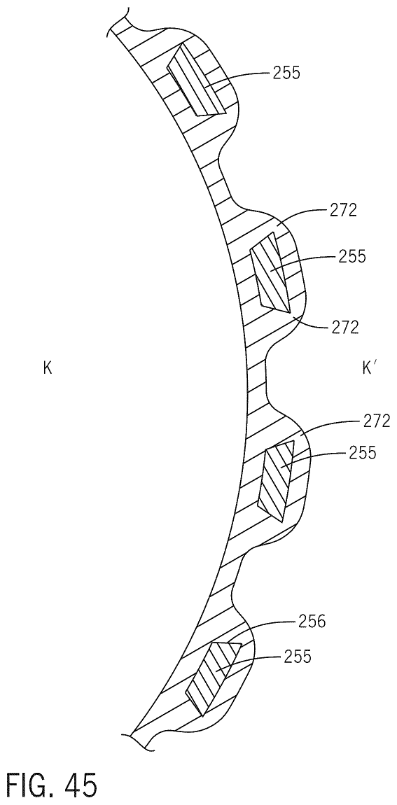

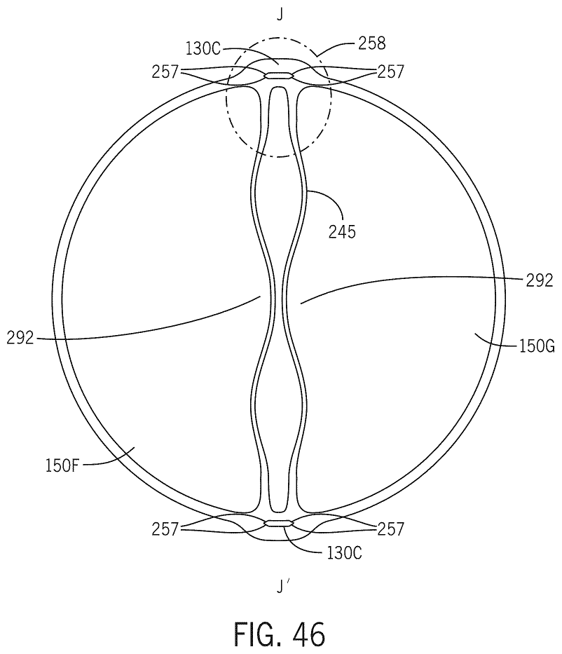

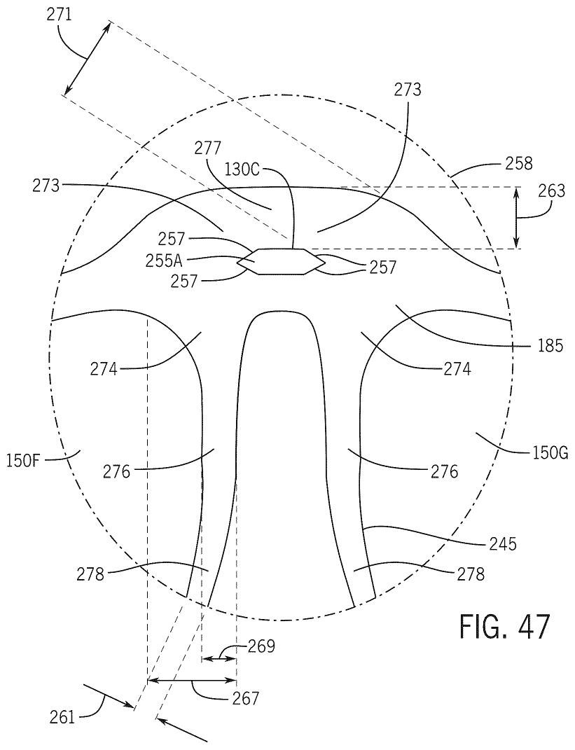

FIG. 42 is a first side or elevational view illustrating a representative ninth embodiment of a prosthetic venous valve assembly 200F and also illustrating a representative seventh embodiment of a frame and venous valve assembly 105E in accordance with the disclosure herein, with this first side view facing a leaflet 150F of the prosthetic venous valve assembly 200F. FIG. 43 is a second, orthogonal side or elevational view illustrating the representative ninth embodiment of a prosthetic venous valve assembly and also illustrating the representative seventh embodiment of a frame and venous valve assembly 105E in accordance with the disclosure herein, with this second side view offset at ninety-degrees from the first side view and facing a valve commissure support 130C of the prosthetic venous valve assembly 200F. FIG. 44 is partial cross-sectional view (through the I-I′ plane of FIG. 42) illustrating the representative ninth embodiment of the prosthetic venous valve assembly 200F in accordance with the disclosure herein. FIG. 45 is partial cross-sectional view (through the K-K′ plane of FIG. 42) illustrating the representative ninth embodiment of the prosthetic venous valve assembly 200F in accordance with the disclosure herein. FIG. 46 is a partial cut-away view (illustrated distal to the J-J′ plane of FIG. 42) providing a sectional view of the valve edges 245 of the valve leaflets 150F, 150G and of the frame coating 185 encapsulating the valve commissure supports 130C, and illustrating a variable thickness of the valve leaflets 150F, 150G and a variable thickness of the inner frame coating 185 of the representative ninth embodiment of the prosthetic venous valve assembly 200F in accordance with the disclosure herein. FIG. 47 is an enlarged view of a portion of the partial cut-away view (illustrated distal to the J-J′ plane of FIG. 42) illustrating in greater detail the variable thickness of the inner frame coating 185 and the variable thickness of the valve leaflets 150F, 150G of the representative ninth embodiment of the prosthetic venous valve assembly 200F in accordance with the disclosure herein. It should be noted that the features illustrated and described for the prosthetic venous valve assembly 200F are equally applicable to any and all of the prosthetic venous valve assemblies 100, 100A, 200-200F.

Referring to FIGS. 42-47, a representative ninth embodiment of a frame and venous valve assembly 105F and/or a prosthetic venous valve assembly 200F comprises a second, inner valve frame 160E having an (inner) valve lumen 191 and a polymeric, prosthetic venous valve 120D arranged in the lumen 191 and the prosthetic venous valve 120D also having an open state 225 and a partially closed state 230 or a fully closed state 235. The second, inner valve frame 160E comprises one or more metal struts 255, 255A forming a second frame matrix 250E, with the second frame matrix 250E having a second plurality of closed cells 165 and also forming a plurality of elongated valve commissure supports 130C and a plurality of lateral valve supports 260C. The second frame matrix 250E is also arranged to form a plurality of more arcuate or curvilinear inverted “V”, inverted “U”, or triangular shapes, providing openings or gaps 270A in between the plurality of lateral valve supports 260C. The proximal ends of respective lateral valve supports 260C also meet at an apex 265C of the respective diamond (triangular) regions 140A, and are integrally formed with or otherwise coupled to a corresponding, proximally-elongated valve commissure support 130C. Also as illustrated in FIGS. 42 and 43, the valve frame 160E of the prosthetic venous valve assembly 200F optionally also includes one or more locking tabs 195 extending distally from one or more metal struts 255 at the distal end 259 of the second frame matrix 250E. The one or more locking tabs 195 also have a size and configuration structured, adapted or configured to be removably insertable into one or more corresponding or mating keys or slots 345 of a delivery catheter 300, 300A. Not separately illustrated in FIGS. 42 and 43, the valve frame 160E also may also include a plurality of adaptive, closed cell strut extension members 125 extending distally (inflow end), particularly when implemented to be a prosthetic venous valve assembly 200F without any outer frame 110-110D.

The polymeric, prosthetic venous valve 120D comprises a frame coating 185 coupled to or surrounding the second frame matrix 250E; and a plurality of valve leaflets 150, illustrated as valve leaflets 150F and 150G, which are integrally formed with the frame coating 185, with each leaflet 150F, 150G of the plurality of leaflets 150 moveable from the open state 225 to form the partially closed state 230 or the fully closed state 235 and vice-versa. The (inner) frame coating 185 of the polymeric, prosthetic venous valve 120A also comprises one or more polymeric layers conformally encapsulating, adhered to and/or surrounding the valve frame 160E and second frame matrix 250E. Also illustrated in FIGS. 42 and 43, the polymer forming the inner frame coating 185 also may be sufficiently translucent (or thin) in various regions, such that while the frame coating 185 substantially coats and surrounds the second frame matrix 250E, the second frame matrix 250E may nonetheless be visible through the frame coating 185.

The prosthetic venous valve 120D is illustrated as a bi-leaflet venous valve, having two valve leaflets 150F and 150G, which are also integrally formed as a polymeric layer or sheet with and extending from the frame coating 185 within the respective openings or gaps 270A defined by the lateral valve supports 260C and the valve commissure supports 130C of the second frame matrix 250E. The valve leaflets 150F and 150G also are each flexible and moveable within the respective opening or gap 270A, and generally will move correspondingly to the pressure exerted by fluid, such as blood, flowing through the prosthetic venous valve 120D. Each valve leaflet 150F and 150G also terminates in a respective valve edge 245 and at the lateral valve supports 260C and valve commissure support 130C. In a closed state, the respective valve edges 245 meet with and abut one or more valve edges 245 of adjacent valve leaflets 150 to form a corresponding commissure 240 in a fully closed state, similarly to what is illustrated in FIG. 25 for the tri-leaflet valve. In various representative embodiments, the valve leaflets 150F and 150G are also formed, structured, configured, or shaped during fabrication to have sinusoidal or scalloped valve edges 245, for example and without limitation, and may be fabricated to provide varying degrees of valve closure, such as fully or partially closed. The valve leaflets 150F and 150G differ from the other bi-leaflet valve leaflets 150D and 150E insofar as the valve leaflets 150F and 150G have a variable thickness extending from the lateral valve supports 260C and the valve commissure supports 130C, as described in greater detail below.

The frame and venous valve assembly 105F differs from the frame and venous valve assembly 105 in several ways, in addition to having a bi-leaflet rather than tri-leaflet configuration. The second, inner valve frame 160E of the frame and venous valve assembly 105F also further comprises a plurality of commissure support strut (or wire) halos 115 (or, equivalently referred to as commissure support struts 115), with each commissure support strut halo 115 coupled between corresponding, proximally elongated valve commissure supports 130C, such that each end 118 of each commissure support strut halo 115 is coupled to and extends proximally from a corresponding valve commissure support 130C, as illustrated. The commissure support strut halos 115 may be included when the frame and venous valve assembly 105F is to be deployed directly as a prosthetic venous valve assembly 200F without any outer valve frame 110-110D. While illustrated as “wishbone” shaped, the commissure support strut halos 115 also may have any suitable arcuate configuration or shape, and serve to stiffen and stabilize the corresponding valve commissure supports 130C and prevent excessive inward (distal) deflection of the valve leaflets 150F and 150G, as well as maintain the roundness of the valve 120D. The commissure support strut halos 115 may be integrally fabricated with the rest of the second, inner valve frame 160E. The frame and venous valve assembly 105F also illustrates an inverted “V”, inverted “U”, or triangular shapes of the second frame matrix 250E, providing openings or gaps 270A in between the plurality of lateral valve supports 260C and valve commissure supports 130C.

In a representative embodiment, the shape of the lateral valve supports 260C and valve commissure supports 130C (stent boundary) along the leaflet (i.e. the margin of attachment) follows a desired shape to best support the leaflets 150F, 150G to minimize strain when the leaflet 150F, 150G is in the closed position and supporting the backpressure of the blood. For example and without limitation, this may be a curvilinear shape, and it may be different for a bi-leaflet valve versus a tri-leaflet design.

Several additional and significant features of the representative prosthetic venous valve assembly 200F are also illustrated in FIGS. 42-47. First, the metal struts 255 and 255A are configured and/or shaped differently depending upon the region or location of the metal struts 255, 255A within the valve frame 160E. For the prosthetic venous valve assembly 200F, in the regions of the valve frame 160E adjacent to or near the venous valve 120D, such the valve commissure supports 130C and/or the lateral valve supports 260C of the valve frame 160E, the one or more metal struts 255A are further chamfered and/or beveled, having double-beveled or chamfered strut edges 257, forming an overall hexagonal shape, as illustrated in FIG. 44. These double-beveled or chamfered strut edges 257 of the one or more metal struts 255A are utilized in regions of the valve frame 160E in which the frame coating 185 may experience greater stress from movement of the valve leaflets 150F and 150G during use, such as in the regions encapsulating, adjacent to or near the valve commissure supports 130C and/or lateral valve supports 260C. Stated another way, each metal strut 255A forming a valve commissure support 130C has a double-beveled or chamfered strut edge 257 on each of two opposite sides of the metal strut 255A (first side 286 and second side 288 of the prosthetic venous valve assembly 200F), as illustrated. These double-beveled or chamfered strut edges 257 provide for an increased thickness of the frame coating 185 in selected regions of the valve frame 160E, in particular the outer regions 277 and 273 of the frame coating 185 encapsulating the valve commissure supports 130C, which may experience comparatively greater stress from movement of the valve leaflets 150F and 150G during use. In contrast, the frame coating 185 is generally thinner around the outer portions 272 of the single-beveled or trapezoidal-shaped struts 155, 255.

This increased thickness of the frame coating 185 in these outer regions 277 and 273 encapsulating the valve commissure supports 130C thereby reduces the potential for tearing of the valve leaflets 150F and 150G from the movement of the valve leaflets 150F and 150G with blood flow. This increased thickness of the frame coating 185 adjacent or near the valve commissure supports 130C and/or lateral valve supports 260C further provides for greater adherence of the frame coating 185 and valve leaflets 150F and 150G to the valve frame 160E, while the frame coating 185 thickness in other selected regions of the valve frame 160E may be reduced, such as within the plurality of closed cells 165. The comparatively reduced thickness of the frame coating 185 in these other portions of the prosthetic venous valve assembly 100, 100A, 200-200F, such as within the first plurality of closed cells 170 and/or the second plurality of closed cells 165, further allows the prosthetic venous valve assembly 100, 100A, 200-200F to be crimped or compressed onto a catheter 300, 300A for deployment, while also allowing the crimped or compressed prosthetic venous valve assembly 100, 100A, 200-200F to self-expand into its full, deployed state. In the other regions of the valve frame 160E which are not adjacent to or near the venous valve 120D, the one or more metal struts 255 also may have a single-bevel (trapezoidal) shape 256 in cross-section, such as illustrated in FIGS. 7, 8, and 45 and as described previously. In addition to the illustrated single-bevel, trapezoidal-shaped struts 155, 255 and double-beveled or chamfered hexagonal-shaped struts 255A, those having skill in the art will recognize that the struts 155, 255, 255A may have any of a wide variety of other shapes (in cross-section), such as circular, elliptical, square or rectangular, for example and without limitation, and all such variations are considered within the scope of the disclosure. While these double-beveled or chamfered, hexagonal-shaped struts 255A are more labor intensive and thus more expensive to manufacture, additional thickness of the frame coating 185 in the outer regions 277 and 273 encapsulating the valve commissure supports 130C provides advantageous additional support for the repetitive movement of the valve leaflets 150F and 150G. The frame coating 185 in the outer regions 277 and 273 encapsulating the valve commissure supports 130C has been empirically determined to be particularly prone to failure. The struts 255 in other sections of the frame matrix 250E that are less prone to failure may be a single-bevel, trapezoidal-shaped struts, as illustrated. For example, the section of the struts 255 illustrated in FIG. 45 may have a single-bevel, trapezoidal-shape. In addition, the selection of any of the various polymers or combination or mixtures of polymers to form the frame coating 185 may also affect selections of the shapes of the struts 155, 255, 255A and thicknesses of the frame coating 185 in selected regions of the valve frame 160E, also for example and without limitation.

Another significant feature of the prosthetic venous valve assembly 200F is the variable thickness of the valve leaflets 150F and 150G and of the frame coating 185 in different regions of the prosthetic venous valve assembly 200F, to provide many significant features, including, for example and without limitation: (1) resistance against tearing of the venous valve 120D, particularly in the outer regions 277 and 273 of the frame coating 185 encapsulating the valve commissure supports 130C; (2) selected or desired flexibility of the valve leaflets 150F and 150G for opening and closing during blood flow; (3) crimping or compressing of the prosthetic venous valve assembly 100, 100A, 200-200F onto a catheter 300, 300A for deployment; and (4) allowing the crimped or compressed prosthetic venous valve assembly 100, 100A, 200-200F to expand or self-expand into its full, deployed state. The selection of one or more polymers, or combinations or mixtures of polymers, to form the valve leaflets 150F and 150G and the frame coating 185 may also be optimized or “tuned” based upon different polymer characteristics, including the different available durometers of the polymeric materials, such as hardness, tear resistance, flexibility, and stretchability, for example and without limitation.

Apart from or in addition to the polymer selection, as illustrated in FIGS. 46 and 47, the thickness “V” (263) of the frame coating 185 in the outer region 277 and the thickness “W” (271) of the frame coating 185 in the adjacent outer regions 273, which are the outer portions of the frame coating 185 encapsulating, surrounding and/or adjacent to each one of the valve commissure supports 130C (and/or one of the lateral valve supports 260C), is comparatively greater, with the thickness of the frame coating 185 gradually decreasing and tapering to a thinner thickness “Z” (261) in the region 278 of each valve leaflet 150F and 150G. The thickness of the frame coating 185 generally decreases with increasing distance away from the valve commissure support 130C and corresponding lateral valve support 260C, illustrated as gradually decreasing from the thickness “V” (263) of the frame coating 185 in the outer region 277 and the thickness “W” (271) of the frame coating 185 in the adjacent outer regions 273 and transitioning to a thickness “X” (276) in region 274 which is further away from the corresponding valve commissure support 130C and/or lateral valve support 260C, gradually decreasing and transitioning further to a thickness “Y” (269) in region 276 which is still further away from the corresponding valve commissure support 130C and/or lateral valve support 260C, and still further away from the corresponding valve commissure support 130C and/or lateral valve support 260C, finally gradually decreasing and transitioning further to and maintaining comparatively constant a thickness “Z” (261) beginning in region 278 and extending at this generally uniform thickness into the center region 292 of the moveable valve leaflets 150F and 150G (i.e., with thickness “V” (263) of region 277 and thickness “W” (271) of regions 273>thickness “X” (267) of region 274>thickness “Y” (269) of region 276>thickness “Z” (261) of region 278).

Stated another way, the polymeric frame coating 185 has first thicknesses thickness “V” (263) of region 277 and “W” (271) of regions 273 of the outer portions (277, 273) of the frame coating 185 adjacent to and encapsulating each valve commissure support 130C of the plurality of valve commissure supports 130C, with the first thickness “V” and “W” of the polymeric frame coating 185 tapering to a second thickness (“X” (267) or “Y” (269)) spaced apart from each valve commissure support 130C of the plurality of valve commissure supports 130C (in regions 274 and 276), with the second thickness less than the first thickness. In turn, the one or more polymeric valve leaflets 150F and 150G each have at least one third thickness “Z” (261) tapering from the second thickness, the at least one third thickness less than the second thickness.

In a representative embodiment, the variable thicknesses of the frame coating 185 and the valve leaflets 150F and 150G may be described using a mathematical ratio, and the ratio of thicknesses may vary from the greatest thickness in the vicinity of the corresponding valve commissure support 130C (and/or lateral valve support 260C), especially the outer portions (277, 273) of the frame coating 185 adjacent to and encapsulating each valve commissure support 130C, compared to the smallest thickness in the central or center region 292 of each valve leaflet 150 (e.g., valve leaflets 150F and 150G). In a representative embodiment, this thickness ratio of greatest to smallest thickness may be 12:1, or more particularly 10:1, or more particularly 5:1, or more particularly 4:1, or more particularly between 4:1 and 2:1, or more particularly between 5:1 and 1.5:1, or more particularly between 4:1 and 1.5:1, for example and without limitation. Also in a representative embodiment of the prosthetic venous valve assembly 200F, for example and without limitation, depending upon polymer material selection, the thickness of the frame coating 185 adjacent or near the lateral valve supports 260C and the valve commissure supports 130C may vary in a range between 40-250 microns, while the thickness of the frame coating 185 within the plurality of closed cells 165 may vary in a range between 20-80 microns, and the thickness of the valve leaflets 150F and 150G also may vary in a range between 20-80 microns, all for example and without limitation. Also in a representative embodiment of the prosthetic venous valve assembly 200F, for example and without limitation, depending upon polymer material selection, the thickness of the frame coating 185 may vary from 25 microns to 150 microns, such as from the smallest thickness in the central or center region 292 of each valve leaflet 150 to the greatest thicknesses in the outer portions (277, 273) of the frame coating 185 adjacent to and encapsulating each valve commissure support 130C.

It has been determined that the weakest point of the frame coating 185 frequently occurs at the outer regions 273 of the frame coating 185 on the outside of the valve commissure supports 130C. Thus, in order to reduce the chances of tearing or ripping at the portion of the frame coating 185 on the outside of the valve commissure supports 130C, this frame coating 185 at the outer regions 273 on the outside of the valve frame coating 185 may have a thickness “W” (271) of greater than or equal to 25 microns. It has further been determined, however, that a thickness “W” (271) of the outer regions 273 of the frame coating 185 which is less than 150 microns allows the prosthetic venous valve assembly 200F to be crimped, compressed, or otherwise folded to provide for the transcatheter deployment of the prosthetic venous valve assembly 200F, described in greater detail below. Thus, a thickness “W” (271), of the outer regions 273 of the frame coating 185, which is between 25 microns and 150 microns has been determined to be advantageous. Furthermore, the double beveled, hexagonal-shaped struts 255A forming the valve commissure support 130C enables the thickness “W” (271) to be greater (e.g., compared to the thickness in the region 272 illustrated in FIG. 45) without significantly increasing the thickness of the frame coating 185 of the rest of the valve frame 160E of the prosthetic venous valve assembly 200F.

It should be noted that during fabrication, there may be lateral deviations from or variances of these various thicknesses of the frame coating 185, such as lateral deviation or variance of first, second, and/or third thicknesses on left and right of each of the first, second (or third) valve commissure supports 130C (e.g., lateral deviation or variance between the left side (282) and the right side (284) of the prosthetic venous valve assembly 200F illustrated in FIG. 42), lateral deviation or variance side-to-side (e.g., lateral deviation or variance between a first (or front) side 286 (i.e., the first side 286 with the valve leaflet 150F) and a second (or rear) side 288 (i.e., the second side 288 with the valve leaflet 150G) of the prosthetic venous valve assembly 200F illustrated in FIG. 43), and/or both types of lateral deviations or variances of thicknesses (left-right lateral thickness deviation or variance and side-to-side lateral thickness deviation or variance). In representative embodiments, this lateral deviation or variance of the first thickness of the frame coating 185 is generally less than or equal to 20 microns (i.e., between zero and 20 microns). Stated another way, at least one valve commissure support 130C (of the plurality of valve commissure supports 130C) divides the polymeric frame coating 185 into a first side 286 polymeric frame coating and a second side 288 polymeric frame coating, wherein a variation of thickness between corresponding portions of the first side polymeric frame coating and the second side polymer frame coating is less than or equal to 20 microns.

While the thickness tapering of the polymeric frame coating 185 is illustrated in FIGS. 46 and 47 with respect to each valve commissure support 130C of the plurality of valve commissure supports 130C, in representative embodiments, the same or similar thickness tapering is also provided with regard to the polymeric frame coating 185 surrounding or adjacent to each lateral valve support 260C of the plurality of lateral valve supports 260C, and in representative embodiments, the same or similar thickness tapering of the polymeric frame coating 185 is also provided with regard to the polymeric frame coating 185 surrounding the other portions of the valve frame 160E, such as tapering from the thickness encapsulating the struts 255 to a thinner thickness in the center(s) of the closed cells 165. In these representative embodiments, not separately illustrated, the polymeric frame coating 185 also has a sixth thickness “M” surrounding or adjacent to each lateral valve support 260C (with the sixth thickness “M” generally being the same as or close to the first thickness “V” or “W”), with the sixth thickness “M” of the polymeric frame coating 185 surrounding or adjacent to each lateral valve support 260C tapering to a fifth thickness (“N”) spaced apart from each lateral valve support 260C of the plurality of lateral valve supports 260C (with the fifth thickness “N” generally being the same as or close to the second thicknesses (“X” (267) or “Y” (269)), with the fifth thickness “N” less than the sixth thickness “M”. In turn, the one or more polymeric leaflets each have at least one third thickness “Z” (261) also tapering from the fifth thickness “N”, the at least one third thickness “Z” (261) less than the fifth thickness “N”. Also in turn, the polymeric frame coating 185 within the closed cells 165 may have at least one fourth thickness “P”, also tapering from the fifth thickness “N”, the at least one fourth thickness “P” less than the fifth thickness “N”. It should be noted that any of these different thicknesses may be sequentially described as first, second, third, fourth, fifth, etc., depending upon the thicknesses being compared. For example and without limitation, the polymeric frame coating 185 has a first thickness “W” (271) encapsulating an outer portion (277, 273) of each valve commissure support 130C, of the plurality of valve commissure supports, the first thickness “W” (271) of the polymeric frame coating 185 tapering to a second thickness (“X” (267) or “Y” (269)) spaced apart from each valve commissure support 130C of the plurality of valve commissure supports 130C, the second thickness (“X” (267) or “Y” (269)) less than the first thickness “W” (271). The one or more polymeric leaflets 150 each have at least one third thickness “Z” (261) tapering from the second thickness (“X” (267) or “Y” (269)), the at least one third thickness “Z” (261) less than the second thickness (“X” (267) or “Y” (269)); and further, the polymeric frame coating 185 within each closed cell 165, of the plurality of closed cells 165, has at least one fourth thickness “P”, the at least one fourth thickness “P” also being less than the second thickness (“X” (267) or “Y” (269)), (as the second thicknesses (“X” (267) or “Y” (269) are generally the same as or close to the fifth thickness “N”).

Those having skill in the art will recognize that there are innumerable different methods which may be utilized to achieve any of these desired thickness ranges, in addition to polymeric material selection, which may be further optimized for different regions of the prosthetic venous valve assembly 100, 100A, 200-200F. For example and without limitation, polymeric material forming the frame coating 185 and the valve leaflets 150 may be applied in multiple and different stages, with different polymeric materials with different properties (hardness, flexibility, durability, etc.) selected for each deposition stage, such as through spraying or dipping of the valve frame 160-160E with a selected polymer, with unwanted areas removed between such stages, along with varying polymeric fluid dynamics, mandril design, polymer curing positions and movement, and polymer curing times, among other factors.

Another optional feature of the valve frame 160E of the prosthetic venous valve assembly 200F are the proximally-extended valve commissure supports 130C, each of which is integrally formed with or coupled between the corresponding apex 265 of the second frame matrix 250E and the respective commissure support strut halo 115, as illustrated in FIGS. 42 and 43. The proximally-extended valve commissure supports 130C serve to extend the venous valve 120D proximally, and affect the degree of closure of the venous valve 120D, as described in greater detail below, similarly to the selection of the angle “α” 148 of other representative embodiments.

Again referring to FIGS. 6-11, an angle “α” 148 is formed at the apex 265 of the diamond-shaped (or triangular-shaped) region 140, 140A providing the corresponding valve commissure support 130, 130A, 130B. Referring to FIGS. 42 and 43, the valve frame 160E of the prosthetic venous valve assembly 200F generally includes proximally extended or proximally elongated valve commissure supports 130C. Along with other factors such as the leaflet 150 shape, size, and configuration, the degree of the angle “α” 148 and/or the length of the proximally extended/elongated valve commissure supports 130C may affect how well and to what degree the valve leaflets 150A-150G move or collapse in response to pressure differentials, and along with other factors, affects whether the valve edges 245 touch each other and form a commissure 240 or maintain some separation (in a partially closed state), potentially aiding in providing blood flow and washout of blood in the sinus(es) 180, 180A, 180B and avoiding thrombosis. The shape and the size or volume of the sinus(es) 180, 180A, 180B may also influence the degree of washout of the blood from the sinuses during closure of the prosthetic venous valve 120-120D, with a smooth, curvilinear U-shaped, spherical or bulbous sinus 180, 180A, 180B providing significantly improved blood flow and washout compared to a more abrupt V-shaped sinus 180, 180A, 180B which has reduced blood flow and more limited washout at the bottom of the (V-shaped) sinus 180, 180A, 180B. The degree of closure, the closing time, and closing volume of or for the polymeric, prosthetic venous valve 120 are also affected by the material selection for the polymer, the thickness of the valve leaflets 150A-150G, the tension and size of the valve leaflets 150A-150G, and the configuration of the polymeric, prosthetic venous valve 120-120D, such as whether the valve leaflets 150A-150G are fabricated or configured to be comparatively tense or relaxed within the respective openings or gaps 270.

In representative embodiments, the second frame matrix 250 is fabricated to provide that the angle “α” 148 is an acute angle and/or a proximally-extended valve commissure supports 130C is included, while the other factors mentioned above, along with the shape and the size or volume of the sinus(es) 180, 180A, 180B, are selected to generally provide a partially closed state of the polymeric, prosthetic venous valve 120, and provide for significant blood washout and avoiding of blood stagnation within the sinus(es) 180, 180A, 180B. In addition, as illustrated in FIGS. 22A, 22B, 22C, and 22D, using these various factors, among others, for example and without limitation, along with mandrel configuration and design as described in greater detail below, the second, inner valve frame 160-160E and the valve leaflets 150A-150G may be designed, configured, and fabricated to have a closed bias 205, a first neutral bias 210, a second neutral bias 215, and/or an open bias 220, thereby determining the extent or degree of closure of the prosthetic venous valve 120 in response to pressure differentials. The forming position of the leaflets 150 (i.e., bias) may impact the strains in the leaflets 150 during the opening-closing cycle which may impact durability. In addition, it may help valve 120-120D performance under low flow or low backpressures to open or close more efficiently based on the forming bias. For example and without limitation, a closed bias may help the valve close better under low backpressures and reduce reflux. The bias shape also defines the opening/closing kinematics by defining the leaflet 150 bending locations

These various biases which can be created during fabrication for the valve leaflets 150A-150G, including the closed bias 205, the first neutral bias 210, the second neutral bias 215, and the open bias 220, then result in different degrees of closure and blood flow through the prosthetic venous valve 120-120D. FIGS. 26A, 26B, 26C, 26D, and 26E are cross-sectional schematic illustrations showing blood flow through the opening and closing states of a representative second embodiment of the prosthetic venous valve assembly in accordance with the disclosure herein, with FIG. 26A showing blood flow through a prosthetic venous valve assembly in a first partially open state, FIG. 26B showing blood flow through the prosthetic venous valve assembly in a second partially open state, FIG. 26C showing blood flow through the prosthetic venous valve assembly in a fully open state, FIG. 26D showing blood flow through the prosthetic venous valve assembly in a closing or partially closed state, and FIG. 26E showing no blood flow through the prosthetic venous valve assembly in a fully closed state. As illustrated in FIGS. 26C and 26D, a bias may be selected to generally provide a partially closed state of the polymeric, prosthetic venous valve 120 and allow some reflux while providing for blood washout and avoiding blood stagnation within the sinus(es) 180, 180A, 180B, as illustrated with the sinus blood flows 275, 280 showing turbulent or circular flows providing washout between the valve leaflets 150A-150G and the sinus(es) 180, 180A, 180B.

In a representative embodiment, as is the case with the first, outer valve frame 110-110D, the second, inner valve frame 160-160E may include a nickel titanium alloy such as Nitinol, but alternatively may be fabricated from any other suitable material, such as titanium, stainless steel, cobalt chromium (CoCr), another metal, a metallic alloy, carbon fiber, a polymer, and combinations thereof, for example and without limitation. In the various representative embodiments, the outer valve frame 110-110D and the inner valve frame 160-160E each may be fabricated from a metal tubing such as Nitinol as known in the field, such as through laser machining and using heat set tooling and mandrels shaped to provide the various sinuses 180, 180A, 180B, the central frame matrix 135, the second frame matrix 250, the commissure support strut halos 115, the adaptive, closed cell strut extension members 125, etc. A particular advantage of using Nitinol is its property of shape memory, such that a crimped or compressed prosthetic venous valve assembly 100, 100A, 200-200F may be self-expanding into its full, deployed state, as illustrated in FIGS. 1-21 and 42-43.

The valve leaflets 150A-150G, inner frame coating 185, 185A, and central coating 175 may include a biocompatible polymer which is benign and anti-thrombotic, with the polymer selected for the valve leaflets 150A-150G having sufficient longevity over its expected duty cycle to continue to perform sufficiently without requiring repeated replacement. Different polymers and solvents may be utilized to create the selected or desired mechanical properties of the prosthetic venous valve 120-120D, such as polyether, polyether polyol, polyether polyurethane, polyurethane, polyurethane urea, siloxane, polysiloxanes, silicone, silicone rubber, polytetrafluoroethylene (PTFE), expanded polytetrafluoroethylene (ePTFE), polyvinyl alchohol (PVA), siloxane-based polyurethane, silicone polyurethane copolymer, and any copolymers, for example and without limitation. Also for example and without limitation, in a representative embodiment, the valve leaflets 150A-150G, inner frame coating 185, 185A, and central coating 175 comprise at least one polymer selected from the group consisting of: a siloxane poly(urethane-urea) elastomer (such as LifePolymer commercially available from Foldax); a silicone-polyurethane copolymer such as polydimethylsiloxane polyurethane (PDMS-PU) (such as Elast-Eon E5-130, Elast-Eon E5-325, or Elast-Eon E2A commercially available from RUA Life Sciences Plc of Irvine, Ayrshire, Scotland, UK, KA11 5AN); a polyhexamethyleneoxide (PHMO), a poly(dimethylsiloxane) (PDMS), a thermoplastic silicone polyether polyurethane (TSPU) (such as PurSil commercially available from DSM Biomedical of Exton, PA 19341 USA), a polyhedral oligomeric silsesquioxane poly(carbonate-urea) urethane (POSS-PCU), a nanocomposite graphene-PCU (FGO-PCU, a poly(styrene-b-isobutylene-styrene) (SIBS), a poly(styrene-b-4-vinylbenzocyclobutylene-b-isobutylene-b-styrene-b-4-vinylbencocylcobutene) (xSIBS), a thermoplastic silicone polycarbonate polyurethane (TSPCU) (such as CarboSil commercially available from DSM Biomedical), an aliphatic polycarbonate urethane (PCU), a siloxane poly(urethane-urea) (SiPUU), segmented polyether polyurethane (SPU) (such as BioSpan commercially available from DSM Biomedical), a carbonate polyurethane (such as Chronoflex commercially available from AdvanSource Biomaterials Corp. of Wilmington, MA 01887 USA), and combinations thereof. Additional or other polymers which may be utilized equivalently are described in greater detail below.

The central coating 175 may be fabricated by spraying or coating a selected polymer over a selected area of the outer valve frame 110-110D, or by dipping the outer valve frame 110-110D in the selected polymer, and also may be shaped using a mandrel, followed by trimming and other finishing steps.

The valve leaflets 150A-150G and inner frame coating 185, 185A also may be fabricated by dipping, spraying or coating a selected polymer over a selected area of the inner valve frame 160-160D, or by dipping the inner valve frame 160-160D in the selected polymer, and forming and shaping the valve leaflets 150A-150G using a mandrel, also followed by cutting, trimming and other finishing steps to remove any unwanted or undesirably polymer or other polymeric substrate from the various frames and or valves, as indicated above. In this fabrication method, there is no separate attachment of the valve leaflets 150A-150G to the inner valve frame 160-160D. Rather, as previously described, the polymer surrounds the inner valve frame 160-160D on both the interior and exterior, encapsulating or embedding the inner valve frame 160-160D within the polymer of the prosthetic venous valve 120-120D to form a frame and venous valve assembly 105-105F. Using a mandrel having a corresponding configuration or shape, the valve leaflets 150A-150G may be integrally formed to have a selected bias to then provide a corresponding, selected level of closure of the prosthetic venous valve 120-120D during operation or deployment, such as to provide a fully open state 225 (as illustrated in FIG. 23), a partially closed state 230 (as illustrated in FIG. 24), or a fully closed state 235 (as illustrated in FIG. 25), as described above. For example and without limitation, the valve leaflets 150A-150G may be formed to have a closed bias 205 illustrated in FIG. 22A, a first neutral bias 210 illustrated in FIG. 22B, a second neutral bias 215 illustrated in FIG. 22C, and an open bias 220 illustrated in FIG. 22D, as examples for the tri-leaflet configurations of the representative first embodiment of the prosthetic venous valve 120 in accordance with the disclosure herein.

As another alternative, similarly to the representative method of fabricating a prosthetic venous valve assembly 200-200F, a prosthetic venous valve 120-120D (both the valve leaflets 150A-150G and inner frame coating 185, 185A) also may be fabricated separately from the inner valve frame 160-160E, such as by using a mandrel dipped or coated with a polymer, and then subsequently attached or otherwise coupled to the inner valve frame 160-160E, such as by inserting the formed prosthetic venous valve 120-120D into the interior of the inner valve frame 160-160E and using a syringe application of the polymer or an adhesive to the exterior of the inner valve frame 160-160E to adhere or bond the prosthetic venous valve 120-120D to the inner valve frame 160-160E, followed by curing the polymer, also for example and without limitation.

The polymeric, prosthetic venous valve 120-120D may be sized depending on the target deployment location. Multiple valve sizes may be required to treat a range of vein size for the patient population being treated. In a representative embodiment, for example and without limitation, the polymeric, prosthetic venous valve 120-120D may have a diameter between 10-14 mm and wherein a thickness of each leaflet 150A-150G of the plurality of leaflets 150A-150G may be between 30-40 microns. In another representative embodiment, also for example and without limitation, the polymeric, prosthetic venous valve 120-120D may have a diameter between 5-14 mm and a thickness of each leaflet 150A-150G of the plurality of leaflets 150A-150G may be between 20-80 microns.

As mentioned above, the various embodiments of the prosthetic venous valve assembly 100, 100A, 200-200F may be delivered and implanted using minimally invasive techniques. In representative embodiments, the prosthetic venous valve assembly 100, 100A, 200-200F is radially collapsible and expandable, and can be collapsed and/or crimped onto a delivery catheter 300, 300A, navigated through a patient's venous vasculature, and expanded (and separated from the delivery catheter 300, 300A) before or during implantation in either a native venous valve site or another region of an affected vein.

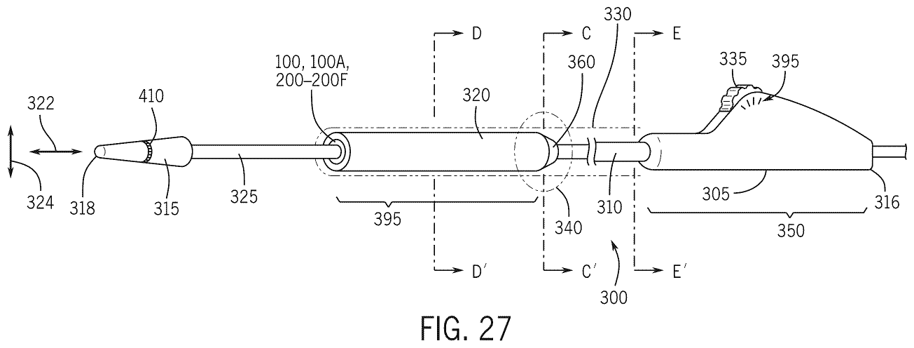

FIG. 27 is an isometric view illustrating a representative first embodiment of a delivery catheter 300 having a prosthetic venous valve assembly 100, 100A, 200-200F in its crimped or compressed state (or configuration) 405 (within a capsule 395) in accordance with the disclosure herein. FIG. 28 is an isometric view illustrating a representative second embodiment of a delivery catheter 300A having a prosthetic venous valve assembly 100, 100A, 200-200F in its crimped or compressed state (or configuration) 405 (within a capsule 395) in accordance with the disclosure herein. As described in greater detail below, the first and second embodiments of the delivery catheters 300, 300A differ only insofar as the delivery catheter 300 includes a first embodiment of a deployment control handle 350 and the delivery catheter 300A includes a second embodiment of a deployment control handle 390, and are otherwise identical. Each of the delivery catheters 300, 300A may be referred to equivalently as a delivery catheter assembly.

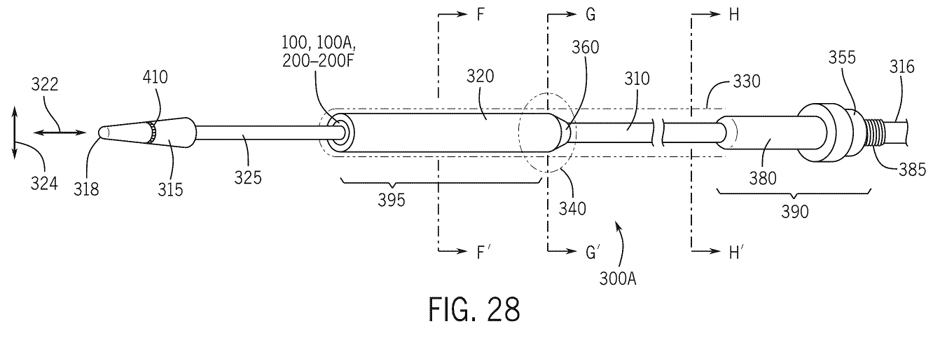

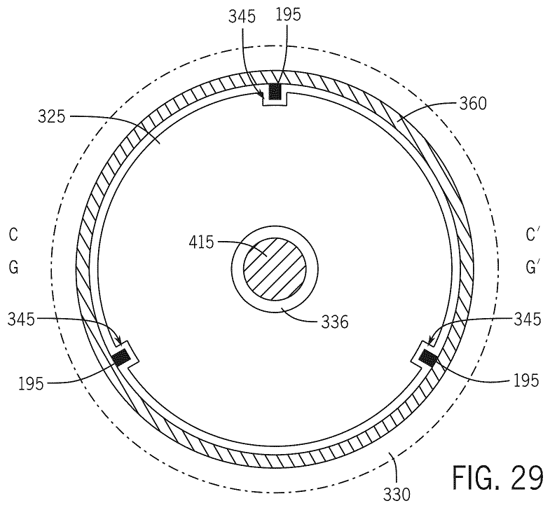

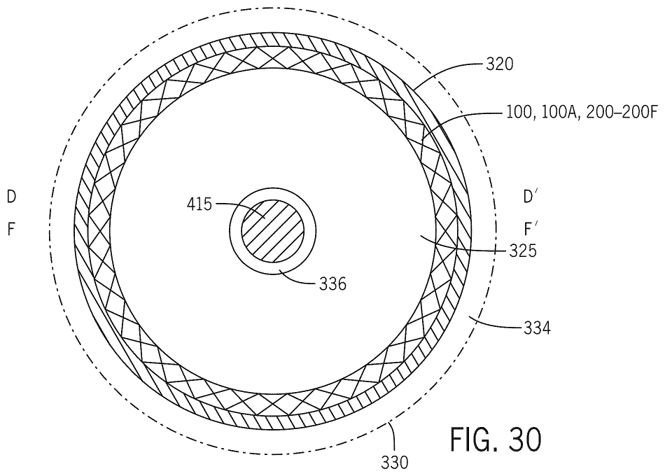

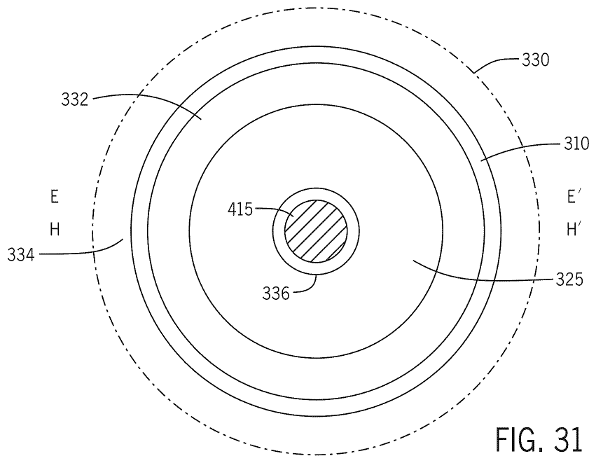

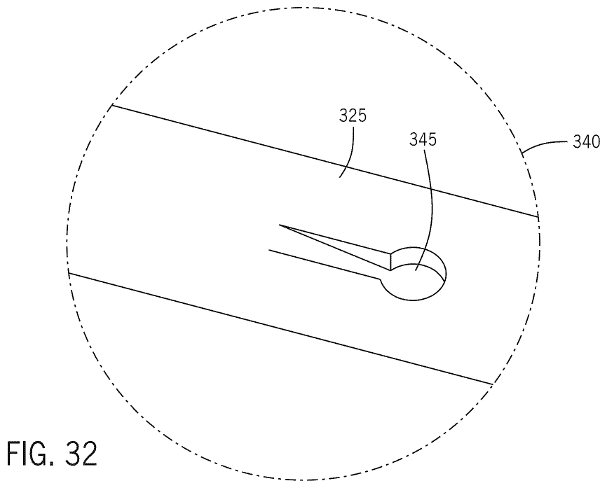

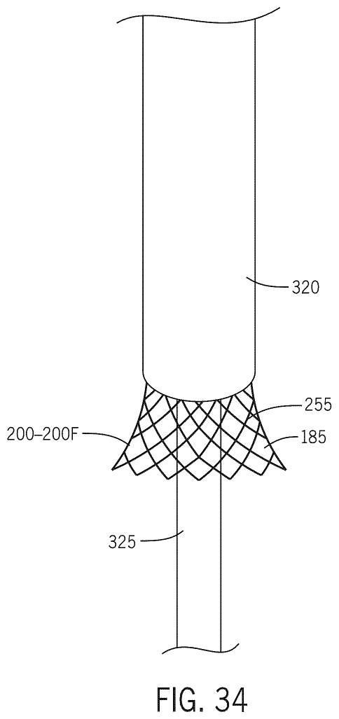

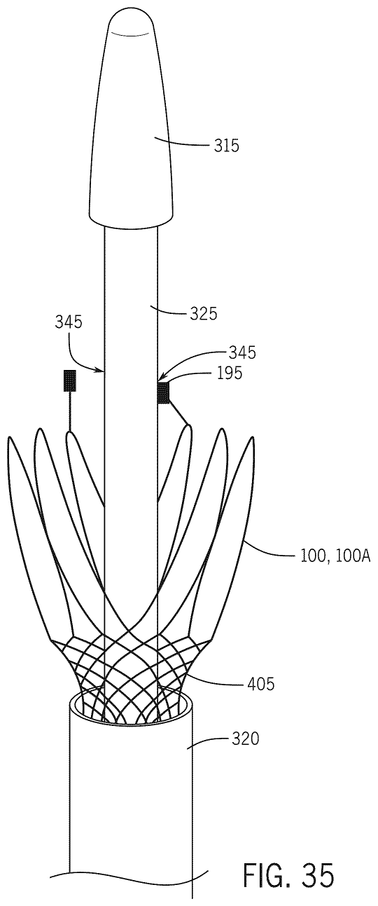

FIG. 29 is a cross-sectional view (through the C-C′ plane and D-D′ plane of FIGS. 27 and 28, respectively) of the representative first and second embodiments of the delivery catheter 300, 300A having the prosthetic venous valve assembly 100, 100A, 200-200F in its crimped or compressed state (or configuration) 405 in accordance with the disclosure herein. FIG. 30 is a cross-sectional view (through the D-D′ plane and F-F′ plane of FIGS. 27 and 28, respectively) of the representative first and second embodiments of the delivery catheter 300, 300A having the prosthetic venous valve assembly 100, 100A, 200-200F in its crimped or compressed state (or configuration) 405 in accordance with the disclosure herein. FIG. 31 is a cross-sectional view (through the E-E′ plane and H-H′ plane of FIGS. 27 and 28, respectively) of the representative first and second embodiments of the delivery catheters in accordance with the disclosure herein. FIG. 32 is a partial isometric, cut-away view illustrating a representative key or slot 345 of the delivery catheter 300, 300A for removable insertion of a locking tab 195 of a prosthetic venous valve assembly 100, 100A, 200-200F in accordance with the disclosure herein. FIG. 33 is a partial isometric, cut-away view illustrating the representative locking tab 195 of a prosthetic venous valve assembly 100, 100A, 200-200F removably inserted into the key or slot 345 of the delivery catheter 300, 300A in accordance with the disclosure herein. FIG. 34 is a partial isometric view illustrating a prosthetic venous valve assembly 200-200F partially expanding from a crimped or compressed state 405 as a capsule cover 320 of the delivery catheter 300, 300A is partially retracted for removable insertion of a prosthetic venous valve assembly in accordance with the disclosure herein. FIG. 35 is a partial isometric view illustrating a prosthetic venous valve assembly 100, 100A (having locking tabs 195 arranged proximally) partially expanding from a crimped or compressed state 405 as the capsule cover 320 of the delivery catheter 300, 300A is partially retracted and the locking tabs 195 are released from keys or slots 345 for removable insertion of a prosthetic venous valve assembly in accordance with the disclosure herein. FIG. 36 is a partial isometric view illustrating a prosthetic venous valve assembly 200-200F (having locking tabs 195 arranged distally) continuing to expand from a crimped or compressed state 405 as the capsule cover 320 of the delivery catheter 300, 300A is fully retracted and the locking tabs 195 are released from keys or slots 345 for removable insertion of a prosthetic venous valve assembly in accordance with the disclosure herein.

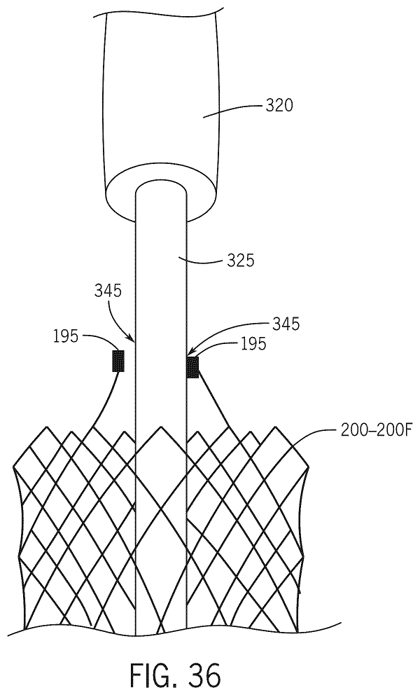

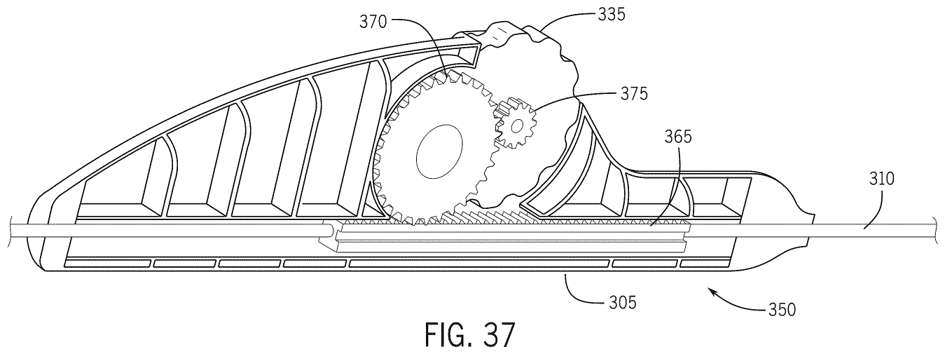

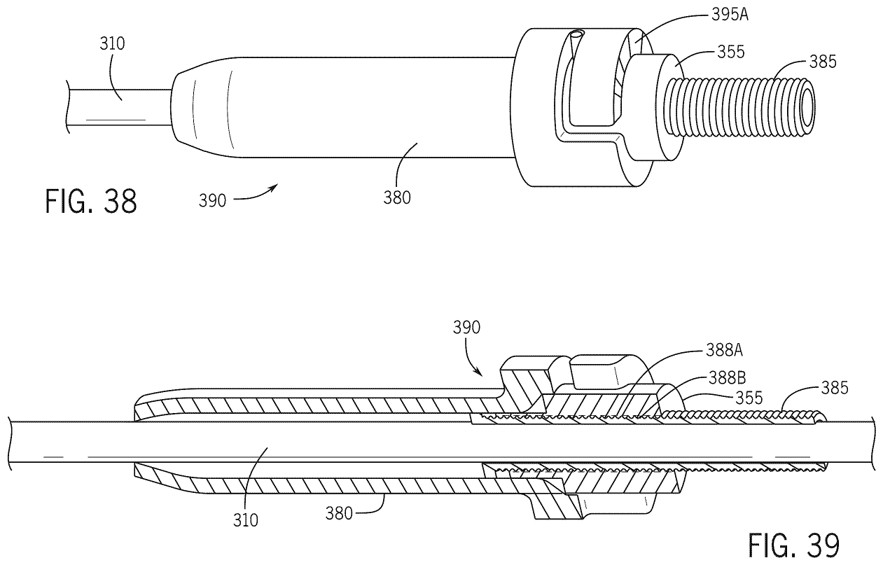

FIG. 37 is a cut-away view illustrating a representative first embodiment of a deployment control handle 350 of a delivery catheter 300 in accordance with the disclosure herein. FIG. 38 is an isometric view illustrating a representative second embodiment of a deployment control handle 390 of the delivery catheter 300A in accordance with the disclosure herein. FIG. 39 is a cut-away view illustrating the representative second embodiment of the deployment control handle 390 of the delivery catheter 300A in accordance with the disclosure herein.

Referring to FIGS. 27-39, the delivery catheter 300, 300A comprises: a capsule 395 (enclosing the prosthetic venous valve assembly 100, 100A, 200-200F in its crimped or compressed state (or configuration) 405); a plurality of coaxial, flexible shafts (namely, a first, inner shaft 325, a second, hollow shaft or tube 310, and optionally a third, outer (and hollow) shaft or tube 330); a guiding (or nose) cone 315 (as a smooth atraumatic molded guiding cone or molded end cap, which may be optional) arranged at the distal end 318 of the delivery catheter 300, 300A; and either a deployment control handle 350 or a deployment control handle 390 arranged at the proximal end 316 of the respective delivery catheter 300, 300A. The guiding cone 315 has a conical shape and a smooth surface for insertion of the delivery catheter 300, 300A into an affected vein. The optional third, outer (and hollow) shaft 330 (illustrated with dashed lines) is coaxial with the first, inner shaft 325 and second shaft 310 and is utilized to isolate or otherwise protect the capsule 395 (enclosing the prosthetic venous valve assembly 100, 100A, 200-200F) as the delivery catheter 300, 300A is inserted through a delivery sheath and a hemostasis valve (not separately illustrated) into the affected vein. The various first, inner shaft 325, second shaft 310, and optional third shaft 330 may be flexible and are comprised of a suitable flexible polymer, as known in catheter field, any and all of which are considered equivalent and within the scope of the disclosure.

Not separately illustrated, the delivery catheter 300, 300A may also be further structured or otherwise configured for use with a catheter guide wire, as known in the catheter field. In addition, the delivery catheters 300, 300A are considered to be oriented in the longitudinal dimension 322, i.e., longitudinally, with the transverse (or radial) dimension 324 being orthogonal to the longitudinal dimension 322, e.g., the capsule 395 both extends in the transverse (or radial) dimension 324 from the first, inner shaft 325 and extends in the longitudinal dimension 322 along a segment of the first, inner shaft 325.

Also as illustrated in FIGS. 27 and 28, as an option, the delivery catheter 300, 300A may also be further structured or otherwise configured to have or to use with intravascular ultrasound (ultrasonic) imaging. For example and without limitation, the delivery catheter 300, 300A may further comprise an array of a plurality of ultrasound transducers 410 and an electrical cable having a plurality of signal (e.g., ultrasound transmit control data or signal wires and ultrasound receive signal wires), power, and ground wires 415 (or, equivalently, an electrical cable having a plurality of signal, power, and ground wires 415) coupled to the array of the plurality of ultrasound transducers 410. Continuing with the example and also without limitation, the guiding (or nose) cone 315 may be arranged and structured or otherwise configured to have the array of the ultrasound transducers 410 for concurrent, real-time ultrasound imaging, and the first, inner shaft 325 may be hollow, having a first shaft lumen 336, and structured or otherwise configured to have the plurality of signal, power and ground wires 415 arranged and extending longitudinally within the first shaft lumen 336 of the first, inner shaft 325.

Referring to FIGS. 27, 28, and 31, the first, inner shaft 325 is arranged coaxially or concentrically within a second shaft lumen 332 of the second, hollow shaft or tube 310. The second, hollow shaft or tube 310 is moveable longitudinally (or longitudinally and rotatably) (e.g., push-pull with or without rotation) with respect to the first, inner shaft 325 (or, equivalently, vice-versa). When the third, outer (and hollow) shaft or tube 330 is included, the first, inner shaft 325 and the second, hollow shaft or tube 310 (with the first, inner shaft 325 arranged coaxially or concentrically within the second shaft lumen 332 of the second, hollow shaft or tube 310) are together arranged coaxially or concentrically within a third shaft lumen 334 of the third, outer (and hollow) shaft or tube 330. The third, outer (and hollow) shaft or tube 330 is moveable longitudinally (or longitudinally and rotatably) (e.g., push-pull with or without rotation) with respect to the first, inner shaft 325 and the second, hollow shaft or tube 310 (or vice-versa).

The capsule 395 is arranged coaxially around the first, inner shaft 325 and is coupled to and moveable (slidably moveable and/or slidably and rotatably moveable) together with the second shaft 310. The capsule 395 comprises a capsule cover 320 and a capsule locking ring 360. The capsule locking ring 360 is arranged in between the capsule cover 320 and the second shaft 310, and the capsule locking ring 360 is coupled to the capsule cover 320 and coupled to and moveable with the second shaft 310. The second shaft 310 is slidably (and/or slidably and rotatably) moveable with respect to the first, inner shaft 325. For deployment of the prosthetic venous valve assembly 100, 100A, 200-200F, using the deployment control handle 350 or the deployment control handle 390, the second shaft 310 is retracted (moved distally) with respect to the first, inner shaft 325, such that the second shaft 310 pulls on the capsule 395, correspondingly retracting the coupled capsule cover 320 and the capsule locking ring 360, thereby exposing the prosthetic venous valve assembly 100, 100A, 200-200F, which may then be expanded or self-expanded (and separated from the delivery catheter 300, 300A) for implantation in either a native venous valve site or another region of an affected vein. When the third, outer (and hollow) shaft 330 is also included, it is similarly retracted to uncover the capsule 395. The capsule cover 320 may be flexible and is comprised of a suitable flexible polymer, and the capsule locking ring 360 may be rigid or flexible and also comprised of a suitable polymer, any and all of which are considered equivalent and within the scope of the disclosure.

While the delivery catheter 300, 300A is illustrated in FIGS. 27 and 28 with one capsule 395 (enclosing one prosthetic venous valve assembly 100, 100A, 200-200F), multiple capsules 395 (with each capsule 395 enclosing a single respective prosthetic venous valve assembly 100, 100A, 200-200F) may be provided on a single delivery catheter 300, 300A, such as for deployment and implantation of multiple prosthetic venous valve assemblies 100, 100A, 200-200F within a single vein or within multiple veins. For these embodiments, the prosthetic venous valve assemblies 100, 100A, 200-200F are arranged within multiple capsules 395, which are spaced-apart longitudinally from each other on the delivery catheter 300, 300A (not separately illustrated), such that the prosthetic venous valve assembly 100, 100A, 200-200F will be in the proper longitudinal direction or orientation within the subject vein once deployed. For example and without limitation, a delivery catheter 300, 300A having a plurality of capsules 395 (each enclosing a prosthetic venous valve assembly 100, 100A, 200-200F) may be inserted into a left popliteal vein of a left leg, and guided through the left femoral vein, the iliac vein, and into the right femoral vein of the right leg for deploying a first prosthetic venous valve assembly 100, 100A, 200-200F (by retracting a first capsule 395 with the retraction of the second shaft 310), followed by retracting the delivery catheter 300, 300A into the left femoral vein of the left leg and deploying a second prosthetic venous valve assembly 100, 100A, 200-200F (by retracting a second capsule 395 with the further retraction of the second shaft 310). Continuing with the example, the first prosthetic venous valve assembly 100, 100A, 200-200F for insertion into the right femoral vein will have a first longitudinal orientation on the delivery catheter 300, 300A for proper blood flow once deployed into the right femoral vein, while the second prosthetic venous valve assembly 100, 100A, 200-200F for insertion into the left femoral vein will have a second, opposite longitudinal orientation on the delivery catheter 300, 300A for proper blood flow once deployed into the left femoral vein.

Also for example and without limitation, when multiple prosthetic venous valve assemblies 100, 100A, 200-200F are to be deployed in the same vein, they may have the same longitudinal orientation, but may have different radial or transverse orientations, e.g., may be offset ninety degrees radially (rotationally) from each other, such as to mimic the arrangement of natural venous valves. Also for example and without limitation, when multiple prosthetic venous valve assemblies 100, 100A, 200-200F are to be deployed, the prosthetic venous valve assemblies 100, 100A, 200-200F may have the same or different sizes, such as to be configured and sized appropriately for the deployed locations with one or more veins.

Referring to FIGS. 29-33, the first, inner shaft 325 of each delivery catheter 300, 300A further comprises one or more keys or slots 345 in the first, inner shaft 325. Each key or slot 345 is a recess, pocket or hole in the first, inner shaft 325 sized, shaped, or otherwise configured for removable insertion of corresponding locking tabs 195 of the prosthetic venous valve assembly 100, 100A, 200-200F. In various representative embodiments, the keys or slots 345 are sized and shaped to have a mating configuration with the locking tabs 195, like a hand and glove, such that a locking tab 195 is removably insertable into and stably held within a key or slot 345, and secured in place by the capsule locking ring 360 of the capsule 395. Stated another way, prior to deployment, the capsule locking ring 360 is arranged to cover and secure the locking tabs 195 within the corresponding keys or slots 345 of the first, inner shaft 325. A wide variety of other sizes and shapes of the locking tabs 195 and mating keys or slots 345 may be implemented, and all such variations are considered equivalent and within the scope of the disclosure. As mentioned above, each locking tab 195 of the prosthetic venous valve assembly 100, 100A, 200-200F is inserted into a corresponding key or slot 345 of the first, inner shaft 325 to secure the prosthetic venous valve assembly 100, 100A, 200-200F on the delivery catheter 300, 300A. The prosthetic venous valve assembly 100, 100A, 200-200F is compressed or crimped into the crimped or compressed state (or configuration) 405 as illustrated in FIGS. 34-36, and covered by the capsule 395 (as illustrated in FIG. 30), with the capsule locking ring 360 covering and securing each locking tab 195 within the corresponding key or slot 345 (as illustrated in FIG. 29). When the second shaft 310 and capsule 395 are retracted, the capsule locking ring 360 is moved to uncover the locking tabs 195 within the keys or slots 345. As the prosthetic venous valve assembly 100, 100A, 200-200F is expanded, the locking tabs 195 are released from the keys or slots 345 and the prosthetic venous valve assembly 100, 100A, 200-200F may be deployed.

While illustrated in FIGS. 27 and 28 with the capsule locking ring 360 arranged distally on the capsule 395, other configurations are possible and considered equivalent and within the scope of the disclosure. In addition, for example and without limitation, while one capsule locking ring 360 is illustrated, a second capsule locking ring 360 may also be utilized at the opposite end of the capsule cover 320. It should be noted that the locking tabs 195 may be arranged either distally or proximally (or both distally or proximally) on the prosthetic venous valve assembly 100, 100A, 200-200F, and the keys or slots 345 and the capsule locking ring 360 are then configured and arranged correspondingly with respect to the first, inner shaft 325 of the delivery catheter 300, 300A, also for example and without limitation. The distal or proximal arrangement of the capsule locking ring 360 on the capsule 395 may depend upon the size and configuration of the capsule locking ring 360 (i.e., whether it can slide over the crimped or collapsed prosthetic venous valve assembly 100, 100A, 200-200F arranged on the first, inner shaft 325 as the second shaft 310 is retracted).

As mentioned above, the deployment control handle 350, 390 is utilized both to advance and to retract the second shaft 310 and capsule 395, with respect to the first, inner shaft 325, to respectively cover and uncover the prosthetic venous valve assembly 100, 100A, 200-200F in the crimped or compressed state (or configuration) 405. Referring to FIGS. 27 and 37, the deployment control handle 350 implements a rack and pinion system to control the advancement and retraction of the second shaft 310 and capsule 395. The deployment control handle 350 comprises a housing 305, a linear (rack) gear 365 arranged within the housing 305, a circular (pinion) gear 370 arranged within the housing 305, a drive gear 375 arranged within the housing 305 and engaged with the circular (pinion) gear 370, and a drive grip (or dial) 335 partially arranged within the housing 305 and coupled to the drive gear 375. The linear (rack) gear 365 is engaged with the circular (pinion) gear 370 and is further coupled to the second shaft 310. The linear (rack) gear 365 is moveable linearly in the longitudinal direction within the housing 305, to advance or retract the second shaft 310, in response to or in conjunction with the rotation of the circular (pinion) gear 370 within the housing 305. The rotation of the circular (pinion) gear 370 is controlled by the drive gear 375 coupled to the drive grip (or dial) 335.

A portion of the drive grip (or dial) 335 is external to or otherwise outside of the housing 305 as illustrated, and is sized and shaped and structured or otherwise configured to be moveable in response to the movement of a user's thumb when holding the deployment control handle 350. As the user rotates the drive grip (or dial) 335, the circular (pinion) gear 370 is rotated correspondingly, engaging with and moving the linear (rack) gear 365 longitudinally which, in turn, advances or retracts the second shaft 310 to cover or uncover the prosthetic venous valve assembly 100, 100A, 200-200F. The housing 305 and drive grip (or dial) 335 may also include corresponding indicia 395 as calibrated deployment indicators, so that the user (e.g., medical personnel) will have a visual and calibrated indicator on the delivery catheter 300 of the state or degree of deployment of the prosthetic venous valve assembly 100, 100A, 200-200F. It should be noted that as the second shaft 310 is retracted, the proximal portion of the second shaft 310 may extend proximally from and through the deployment control handle 350, as illustrated in FIGS. 27 and 37.

Referring to FIGS. 28 and 38-39, the deployment control handle 390 implements a lead screw and rotating collar system to control the advancement and retraction of the second shaft 310 and capsule 395. The deployment control handle 390 comprises a housing 380, a lead screw 385 arranged within the housing 380, and a rotating collar 355 partially arranged within the housing 380 and rotatably coupled to the lead screw 385. The rotating collar 355 and lead screw 385 having mating or matching threads 388A and 388B, respectively. The lead screw 385 is engaged with the rotating collar 355 and is further coupled to the second shaft 310. The lead screw 385 is moveable linearly in the longitudinal direction within the housing 380, to advance or retract the second shaft 310, in response to or in conjunction with the rotation of the rotating collar 355 within the housing 380. The rotation of the rotating collar 355 is controlled by the user.

A portion of the rotating collar 355 is external to or otherwise outside of the housing 380 as illustrated, and is sized and shaped and structured or otherwise configured to be moveable in response to the rotational movement of a user's hand when holding the deployment control handle 390. As the user rotates the rotating collar 355, the threads 388A are rotated correspondingly, engaging with the mating threads 388B and moving the lead screw 385 longitudinally which, in turn, advances or retracts the second shaft 310 to cover or uncover the prosthetic venous valve assembly 100, 100A, 200-200F. The housing 380 and rotating collar 355 also may also include corresponding indicia 395A as calibrated deployment indicators, so that the user (e.g., medical personnel) will have a visual and calibrated indicator on the delivery catheter 300A of the state or degree of deployment of the prosthetic venous valve assembly 100, 100A, 200-200F. It should be noted that as the second shaft 310 is retracted, the proximal portion of the second shaft 310 may extend proximally from and through the deployment control handle 390, as illustrated in FIG. 28.

The various components of the delivery catheters 300A, 300B and the deployment control handles 350, 390, such as the housings 305, 380, the rotating collar 355, the lead screw 385, the linear (rack) gear 365, the circular (pinion) gear 370, the drive gear 375, the drive grip (or dial) 335, the first, inner shaft 325, the second, hollow shaft or tube 310, the third, outer (and hollow) shaft or tube 330, the capsule 395, and the guiding (or nose) cone 315, may be comprised of any suitable material(s), and may be various different materials, including for example and without limitation, suitable polymers or other plastics, metals, alloys, carbon fiber, combinations thereof, and so on, as described in greater detail below.

Alternative systems may also be utilized for the deployment of the prosthetic venous valve assembly 100, 100A, 200-200F. For example and without limitation, instead of using the locking tabs 195 and corresponding keys or slots 345, a prosthetic venous valve assembly 100, 100A, 200-200F may be secured to a delivery catheter using sutures or other tethers, which are then cut, severed, or removed for deployment, not separately illustrated.

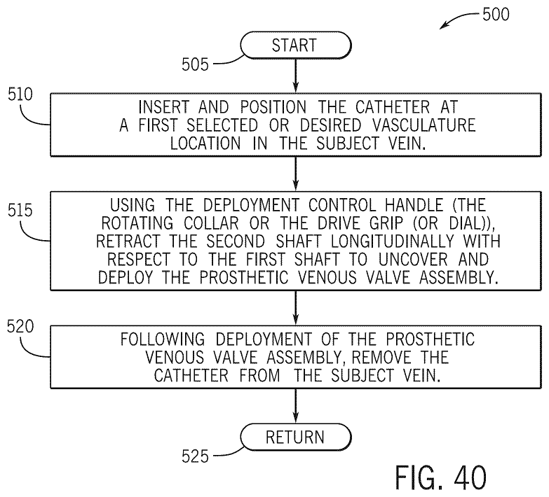

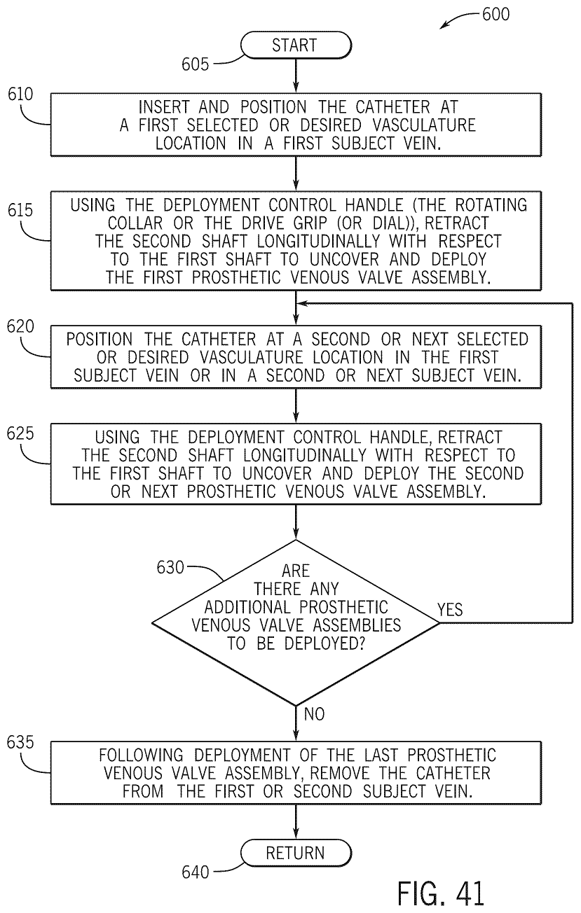

FIGS. 40 and 41 are flow charts illustrating representative first and second method 500, 600 embodiments of using a representative delivery catheter 300A, 300B for deployment of a prosthetic venous valve assembly in a subject vein in accordance with the disclosure herein, and differ insofar as the second method is for deployment of multiple prosthetic venous valve assemblies in a first or second subject vein. In both method embodiments, a first prosthetic venous valve assembly 100, 100A, 200-200F or a first prosthetic venous valve assembly 100, 100A, 200-200F and a second prosthetic venous valve assembly 100, 100A, 200-200F are each configured to self-expand and release from the first shaft 325.

Referring to FIG. 40, the method 500 begins, start step 505, with the user (such as medical personnel) inserting and positioning the delivery catheter 300A, 300B at a first selected or desired vasculature location in the subject vein (a location such as illustrated in FIG. 17), step 510. Using the deployment control handle 350, 390, such as the drive grip (or dial) 335 or the rotating collar 355, the second shaft 310 is then retracted longitudinally with respect to the first shaft 325 to uncover and deploy the prosthetic venous valve assembly 100, 100A, 200-200F, step 515. Following deployment of the prosthetic venous valve assembly, the user may then remove the catheter from the subject vein, step 520, and following application of bandages, etc., the method may end, return step 525.

Referring to FIG. 41, the method 600 begins, start step 605, with the user (such as medical personnel) inserting and positioning the delivery catheter 300A, 300B at a first selected or desired vasculature location in a first subject vein, step 610. Using the deployment control handle 350, 390, such as the drive grip (or dial) 335 or the rotating collar 355, the second shaft 310 is then retracted longitudinally with respect to the first shaft 325 to uncover and deploy the first prosthetic venous valve assembly 100, 100A, 200-200F, step 615. The catheter is then positioned (or repositioned) at a second or next selected or desired vasculature location in the first subject vein or in a second or next subject vein, step 620. Using the deployment control handle 350, 390, such as the drive grip (or dial) 335 or the rotating collar 355, the second shaft 310 is then retracted longitudinally with respect to the first shaft 325 to uncover and deploy the second or next prosthetic venous valve assembly 100, 100A, 200-200F, step 625. When there are any additional prosthetic venous valve assembly 100, 100A, 200-200F to be deployed, step 630, the method returns to step 620 and iterates. Otherwise, following deployment of the last prosthetic venous valve assembly 100, 100A, 200-200F, the user may then remove the delivery catheter 300A, 300B from the subject vein, step 635, and following application of bandages, etc., the method may end, return step 640.

As mentioned above, the representative embodiments of the prosthetic venous valve assembly 100, 100A 200-200F may have any size (height, width, depth), shape, or form factor suitable for use with intravenous insertion in a human or veterinary subject, and all such variations are considered equivalent and within the scope of the disclosure. In addition, the representative embodiments illustrate different combinations of features and elements, with any and all mixing and matching of any of the various features and elements and any and all combinations of any of the various features and elements are within the scope hereof.

The prosthetic venous valve assembly 100, 100A 200-200F and other components may be fabricated in a wide variety of ways, including integrally formed (e.g., injection molded, 3D printed) or assembled from separate components (e.g., using any suitable fasteners or adhesives, not separately illustrated), and all such variations are considered equivalent and within the scope of the disclosure. The prosthetic venous valve assembly 100, 100A 200-200F and other components may be implemented using any suitable material, and may be opaque or transparent, with suitable materials including any rigid (or semi-flexible) polymer or plastic, such as polyvinylchloride (PVC), polystyrene, polyacrylate, polytetrafluoroethylene (PTFE or Teflon), nylon, polycarbonates, polyesters, carbon fiber, glass, silicone, silicone rubber, a metal, an alloy, etc., for example and without limitation, and all such variations are considered equivalent and within the scope of the disclosure. The prosthetic venous valve assembly 100, 100A 200-200F also may have one or more coatings (not separately illustrated), such as an antibiotic or antimicrobial coating, for example and without limitation.

In addition to the polymers described above, other representative examples of suitable polymers include, but are not limited to, fluorinated polymers or copolymers such as poly(vinylidene fluoride), poly(vinylidene fluoride-co-hexafluoropropene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-cotrimethylene carbonate); poly(phosphoesters); poly(phosphoester urethane); poly(trimethylene carbonate); poly(iminocarbonate); poly(ethylene); and any derivatives, analogs, homologues, congeners, salts, copolymers and combinations thereof.

The polymers may also include, but are not limited to, poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers such as poly(vinyl chloride); poly(vinyl ethers) such as, for example, poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; and any derivatives, analogs, homologues, congeners, salts, copolymers and combinations thereof.

The polymers may further include, but are not limited to, poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and any derivatives, analogs, homologues, congeners, salts, copolymers and combinations thereof.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical or structural coupling, connection or attachment, or adaptation or capability for such a direct or indirect electrical or structural coupling, connection or attachment, including integrally formed components and components which are coupled via or through another component.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

1. A transcatheter, prosthetic venous valve assembly comprising:
a valve frame comprising a plurality of struts forming a frame matrix having a plurality of closed cells and a valve frame lumen, the plurality of struts further forming a plurality of lateral valve supports and a plurality of valve commissure supports;
a polymeric frame coating encapsulating the frame matrix; and
a polymeric venous valve having an open or partially open state and a closed or partially closed state, the polymeric venous valve arranged in the lumen, the polymeric venous valve comprising:
one or more polymeric leaflets integrally formed with or coupled to the polymeric frame coating, each leaflet of the one or more polymeric leaflets moveable between the open or partially open state and the closed or partially closed state.

2. The transcatheter, prosthetic venous valve assembly of claim 1, wherein the polymeric frame coating has a first thickness encapsulating an outer portion of each valve commissure support of the plurality of valve commissure supports, the first thickness of the polymeric frame coating tapering to a second thickness spaced apart from each valve commissure support of the plurality of valve commissure supports, the second thickness less than the first thickness.

3. The transcatheter, prosthetic venous valve assembly of claim 2, wherein the one or more polymeric leaflets each have at least one third thickness tapering from the second thickness, the at least one third thickness less than the second thickness; and wherein the polymeric frame coating within each closed cell of the plurality of closed cells has at least one fourth thickness, the at least one fourth thickness less than the second thickness.

4. The transcatheter, prosthetic venous valve assembly of claim 3, wherein the first thickness is between forty (40) to two hundred fifty (250) microns and the third thickness is between twenty (20) to eighty (80) microns.

5. The transcatheter, prosthetic venous valve assembly of claim 3, wherein a ratio of the first thickness to the third thickness is between 5:1 and 1.5:1.

6. The transcatheter, prosthetic venous valve assembly of claim 1, wherein at least one valve commissure support of the plurality of valve commissure supports divides the polymeric frame coating into a first side polymeric frame coating and a second side polymeric frame coating, wherein a variation of thickness between corresponding portions of the first side polymeric frame coating and the second side polymeric frame coating is greater than or equal to zero (0) microns and less than or equal to twenty (20) microns.

7. The transcatheter, prosthetic venous valve assembly of claim 1, wherein the polymeric frame coating conformally encapsulates or coats both an inner portion and an outer portion of each of the frame matrix, the plurality of lateral valve supports, and the plurality of valve commissure supports.

8. The transcatheter, prosthetic venous valve assembly of claim 7, wherein each valve commissure support of the plurality of valve commissure supports comprises at least one double beveled edge and wherein one or more struts of the plurality of struts comprise at least one single beveled edge.

9. The transcatheter, prosthetic venous valve assembly of claim 8, wherein a thickness of the polymeric frame coating on an outer portion of each valve commissure support of the plurality of valve commissure supports is between 25 microns and 150 microns.

10. The transcatheter, prosthetic venous valve assembly of claim 1, wherein each valve commissure support of the plurality of valve commissure supports extends proximally from a corresponding apex formed by the plurality of lateral valve supports, and wherein each leaflet of the one or more polymeric leaflets is coupled between corresponding valve commissure supports of the plurality of valve commissure supports.

11. The transcatheter, prosthetic venous valve assembly of claim 1, wherein the valve frame further comprises:
a plurality of commissure support strut halos coupled between the corresponding valve commissure supports of the plurality of valve commissure supports, each end of each commissure support strut halo coupled to and extending proximally from a corresponding valve commissure support of the plurality of valve commissure supports.

12. The transcatheter, prosthetic venous valve assembly of claim 1, wherein the valve frame further comprises:
at least one locking tab coupled to or integrally formed with and extending distally from the frame matrix, the at least one locking tab removably insertable into a corresponding key or slot of a delivery catheter.

13. The transcatheter, prosthetic venous valve assembly of claim 1, wherein the plurality of struts further form one or more sinuses, and wherein each leaflet of the one or more polymeric leaflets is arranged and moveable within a corresponding sinus of the one or more sinuses.

14. The transcatheter, prosthetic venous valve assembly of claim 1, wherein each leaflet of the one or more polymeric leaflets has a sinusoidal or scalloped edge for forming a commissure with an adjacent leaflet of the plurality of polymeric leaflets in the closed or partially closed state.

15. The transcatheter, prosthetic venous valve assembly of claim 1, wherein the polymeric valve has a diameter between five (5) to fourteen (14) mm and wherein a thickness of each leaflet of the one or more polymeric leaflets is between twenty (20) to eighty (80) microns.

16. The transcatheter, prosthetic venous valve assembly of claim 1, wherein the one or more polymeric leaflets comprise at least one polymer selected from the group consisting of: a siloxane poly(urethane-urea) elastomer, a silicone-polyurethane copolymer; a polydimethylsiloxane polyurethane (PDMS-PU), a thermoplastic silicone polyether polyurethane (TSPU), a polyhexamethyleneoxide (PHMO), a poly(dimethylsiloxane) (PDMS), a thermoplastic silicone polycarbonate polyurethane (TSPCU), a segmented polyether polyurethane (SPU), a polyhedral oligomeric silsesquioxane poly(carbonate-urea) urethane (POSS-PCU), a nanocomposite graphene-PCU (FGO-PCU, a poly(styrene-b-isobutylene-styrene) (SIBS), a poly(styrene-b-4-vinylbenzocyclobutylene-b-isobutylene-b-styrene-b-4-vinylbencocylcobutene) (xSIBS), a thermoplastic silicone polycarbonate polyurethane (TSPCU), an aliphatic polycarbonate urethane (PCU), a siloxane poly(urethane-urea) (SiPUU), a carbonate polyurethane, a polyether, a polyether polyol, a polyether polyurethane, a polyurethane, a polyurethane urea, a siloxane, a polysiloxanes, silicone, silicone rubber, polytetrafluoroethylene (PTFE), expanded polytetrafluoroethylene (ePTFE), polyvinyl alchohol (PVA), a siloxane-based polyurethane, a silicone polyurethane copolymer, and combinations thereof.

17. A transcatheter, prosthetic venous valve assembly comprising:
a valve frame comprising a plurality of struts forming a frame matrix having a plurality of closed cells and a valve frame lumen, the plurality of struts further forming a plurality of lateral valve supports and a plurality of valve commissure supports, each valve commissure support of the plurality of valve commissure supports comprising at least one double beveled edge;
a polymeric frame coating encapsulating both an inner portion and an outer portion of the frame matrix, the polymeric frame coating having a first thickness encapsulating an outer portion of each valve commissure support of the plurality of valve commissure supports, the first thickness of the polymeric frame coating tapering to a second thickness spaced apart from each valve commissure support of the plurality of valve commissure supports, the second thickness less than the first thickness; and
a polymeric venous valve having an open or partially open state and a closed or partially closed state, the polymeric venous valve arranged in the lumen, the polymeric venous valve comprising:
one or more polymeric leaflets integrally formed with or coupled to the polymeric frame coating, each leaflet of the one or more polymeric leaflets moveable between the open or partially open state and the closed or partially closed state, the one or more polymeric leaflets each having at least one third thickness tapering from the second thickness, the at least one third thickness less than the second thickness.

18. The transcatheter, prosthetic venous valve assembly of claim 17, wherein the first thickness is between 25 microns and 150 microns.

19. The transcatheter, prosthetic venous valve assembly of claim 17, wherein at least one valve commissure support of the plurality of valve commissure supports divides the polymeric frame coating into a first side polymeric frame coating and a second side polymeric frame coating, wherein a variation of thickness between corresponding portions of the first side polymeric frame coating and the second side polymeric frame coating is greater than or equal to zero (0) microns and less than or equal to twenty (20) microns.

20. The transcatheter, prosthetic venous valve assembly of claim 17, wherein each valve commissure support of the plurality of valve commissure supports extends proximally from a corresponding apex formed by the plurality of lateral valve supports, and wherein each leaflet of the one or more polymeric leaflets is coupled between corresponding valve commissure supports of the plurality of valve commissure supports.
